(19) United States
(12) Patent Application Publication
(10) Pub. No.: US 2024/0336595 A1
(43) Pub. Date: Oct. 10, 2024

(54) BIFUNCTIONAL COMPOUNDS FOR DEGRADING BTK WITH DIMINISHED IMID ACTIVITY

(71) Applicant: NURIX THERAPEUTICS, INC., San Francisco, CA (US)

(72) Inventors: Cristiana Guiducci, San Francisco, CA (US); Mark Noviski, San Francisco, CA (US); Janine Powers, San Francisco, CA (US); Ryan Rountree, San Francisco, CA (US); Ying Siow Tan, San Francisco, CA (US)

(21) Appl. No.: 18/579,114

(22) PCT Filed: Jul. 13, 2022
(86) PCT No.: PCT/US2022/037029

§ 371 Date: Jan. 12, 2024

Related U.S. Application Data
(60) Provisional application No. 63/221,905, filed on Jul. 14, 2021.

(51) Int. Cl. C07D 401/14; A61K 31/497; A61K 31/506; A61P 35/00; A61P 37/02

(52) U.S. Cl. CPC C07D 401/14; A61K 31/497; A61K 31/506; A61P 35/00; A61P 37/02

(57) ABSTRACT

This disclosure relates to compounds useful for degrading BTK via a ubiquitin proteolytic pathway with little or no IMiD activity. The description also provides pharmaceutically acceptable compositions comprising said compounds and methods of using the compositions in the treatment of various disease, conditions, or disorders.

### CROSS-REFERENCE TO RELATED APPLICATIONS

This application claims the benefit of U.S. Provisional Application No. 63/221,905, filed Jul. 14, 2021, the contents of which is herein incorporated by reference in its entirety.

### FIELD

This disclosure provides novel bifunctional compounds for proteolytically degrading targeted Bruton's tyrosine kinases (BTK) and methods for treating diseases modulated by BTK. In particular embodiments, the compounds are capable of degrading Bruton's tyrosine kinase with little or no IMiD activity. In particular embodiments, the compounds are useful for methods of treating diseases with longer durations of administration, higher doses, or both.

### BACKGROUND

BTK is a member of the TEC family of kinases and is a crucial signaling hub in the B cell antigen receptor (BCR) pathway. Mutations in BTK result in X-linked agammaglobulinaemia (XLA), in which B cell maturation is impaired, resulting in reduced immunoglobulin production. Hendriks, et al., 2011, Expert Opin Ther Targets 15:1002-1021, 2011. The central role of BTK in B cell signaling and function makes BTK an attractive therapeutic target for B cell malignancies as well as autoimmune and inflammatory diseases. Ibrutinib, a covalent inhibitor of BTK, has been approved to treat chronic lymphocytic leukemia (CLL), mantle cell lymphoma (MCL) rand other B cell malignancies, as well as graft-versus-host disease (GvHD). Miklos, et al., 2017, Blood, 120(21):2243-2250. Currently, ibrutinib and second-generation BTK inhibitors are being investigated for oncology and immune-related indications such as rheumatoid arthritis. Akinleye, et al., 2013, J of Hematolo Oncol. 6:59; Liu, et al., 2011, J Pharm and Exper Ther. 338(1):154-163; Di Paolo, et al., 2011, Nat Chem Biol. 7(1): 41-50.

As an alternative to stoichiometric inhibition, proteolytic degradation of BTK could have dramatic consequences for B cell function by effectively blocking BCR signaling. Removal of BTK protein would eliminate BTK kinase activity as well as any protein interaction or scaffolding function of BTK. Specific degradation of BTK could be accomplished using heterobifunctional small molecules to recruit BTK to a ubiquitin ligase thus promoting ubiquitylation and proteasomal degradation of BTK. Thalidomide derivatives, such as lenalidomide or pomalidomide, can be used to recruit potential substrates to cereblon (CRBN), a component of a ubiquitin ligase complex. This unique therapeutic approach could present a mechanism of action for interfering with BTK activity and BCR signaling that is distinct from the mechanism of stoichiometric BTK inhibition. Furthermore, this degradative approach could effectively target the C481S mutated form of BTK, a mutation which has been clinically observed and confers resistance to inhibition by ibrutinib. Woyach, et al., 2012, Blood, 120(6): 1175-1184, 2012.

Using degrader compounds to destroy target proteins through CRBN has already led to candidate anti-cancer drugs. Okumura et al., 2020, Pharmaceuticals 13:95. These drugs not only target the cancer cell, but also trigger a strong immune response in part by degrading, for instance, Ikaros and Aiolos, and by increasing IL-2 secretion. The Immunomodulatory imide Drug (IMiD) portion of these compounds is believed to be responsible for the potent immune effect. Together these degrader compounds hinder tumor growth directly and through the immune system. Quach et al., 2010, Leukemia 24:22-32. However, outside of the cancer therapy context, the potent IMiD activity can cause unwanted side effects. These side effects can limit the use of CRBN-binding degrader compounds to cancers and similar malignancies with few therapeutic alternatives. Compounds with reduced or eliminated IMiD activity would be useful for treating at longer duration and/or higher doses thereby providing new therapeutics, including for indications other than cancer.

### SUMMARY

Provided herein are methods of treating or preventing a disease, disorder, or condition in a subject in need thereof by administering a compound capable of degrading Bruton's tyrosine kinase with little or no IMiD activity. In the examples provided herein, compounds are shown to recruit CRBN and degrade BTK with little or no IMiD activity. Specifically, in certain embodiments, exemplary compounds degrade BTK while not promoting degradation of Aiolos or Ikaros. In certain embodiments, embodiments, the compounds also do not trigger IL-2, another marker of IMiD activity. By degrading BTK without significant IMiD activity, the compounds can be tolerated at higher doses with fewer side effects than other degrader compounds that modulate CRBN.

In one aspect, provided herein are methods of treating or preventing a disease, disorder, or condition in a subject in need thereof. The methods comprise the step of administering to the subject an amount of a bifunctional compound capable of inducing proteolytic degradation of Bruton's tyrosine kinase with little or no IMiD activity. In certain embodiments, the amount is effective to treat or prevent the disease, disorder, or condition. In certain embodiments, the methods are for treating or preventing cancer, an autoimmune disease, or an inflammatory disease.

In one aspect, provided herein are methods of treating or preventing a brain disease, disorder, or condition in a subject in need thereof. The methods comprise the step of administering to the subject an amount of a bifunctional compound capable of inducing proteolytic degradation of Bruton's tyrosine kinase with little or no IMiD activity. In certain embodiments, the amount is effective to treat or prevent the brain disease, disorder, or condition. In certain embodiments, the methods are for treating or preventing brain cancer.

In another aspect, provided herein are methods of degrading Bruton's tyrosine kinase in a subject in need thereof. The methods comprise the step of administering to the subject an amount of a bifunctional compound capable of inducing proteolytic degradation of Bruton's tyrosine kinase with little or no IMiD activity. In certain embodiments, the amount is effective to degrade Bruton's tyrosine kinase in the subject.

In another aspect, provided herein are methods of preventing B cell activation in a subject in need thereof. The methods comprise the step of administering to the subject an amount of a bifunctional compound capable of inducing proteolytic degradation of Bruton's tyrosine kinase with little or no IMiD activity. In certain embodiments, the amount is effective to prevent B cell activation.

In another aspect, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase with little or no IMiD activity. In certain embodiments, the amount is effective to degrade the mutant Bruton's tyrosine kinase. In certain embodiments, the mutant Bruton's tyrosine kinase is a C481 mutant. In certain embodiments, the mutant Bruton's tyrosine kinase is a C481 S mutant.

In the methods, the bifunctional compounds comprise a moiety capable of specifically binding BTK with little or no IMiD activity. Particular compounds are described herein. The compounds can be administered in any form, including pharmaceutically acceptable salts and pharmaceutical compositions. In particular embodiments, the compounds are administered orally.

The methods provided herein are useful for treating or preventing diseases, conditions, and disorders mediated by Bruton's tyrosine kinase, including, for instance, cancer, autoimmune conditions, and inflammatory conditions.

### BRIEF DESCRIPTION OF THE FIGURES

FIG. 1 provides the effect of compound 1 on a collagen-induced arthritis model including clinical arthritis score (FIG. 1A), body weight (FIG. 1B), and serum collagen IgG (FIG. 1C).

FIG. 2 provides the effect of compound 1 on an experimental autoimmune encephalomyelitis.

FIG. 3 provides the effects of compound 1 on a systemic lupus erythematosus model including urine protein score (FIG. 3A), DNA titer (FIG. 3B), and glomerulus diameter and histology scores (FIG. 3C).

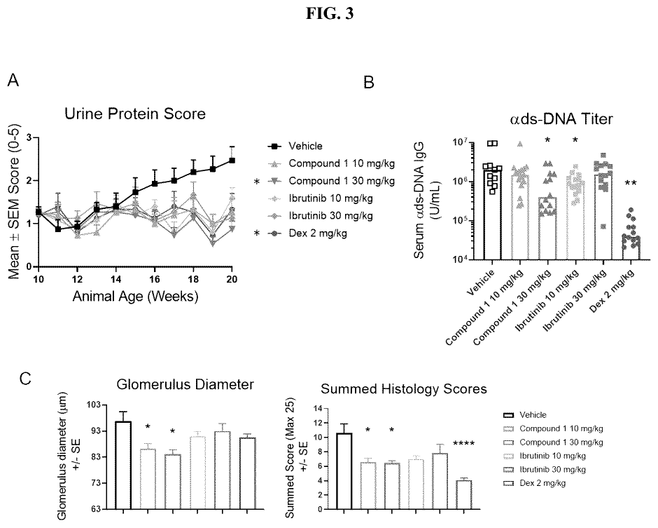

FIG. 4 provides the effects of compound 1 dosing on B cell compartment including BTK degradation (FIG. 4A), B cell percentage (FIG. 4B), and bone marrow plasma cell reduction (FIG. 4C).

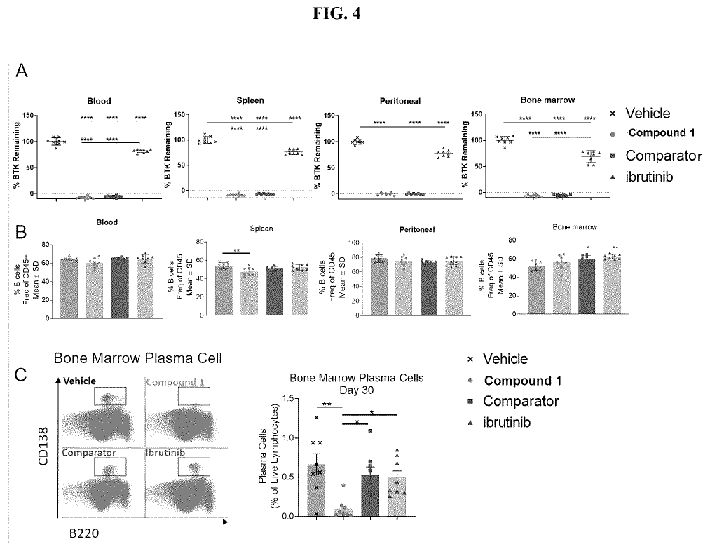

FIG. 5 provides the effects of compound 1 on plasma cell generation on immunization including treatment groups (FIG. 5A), Bruton's tyrosine kinase degradation (FIG. 5B), B cell effects (FIG. 5C), and plasma cell effects (FIG. 5D).

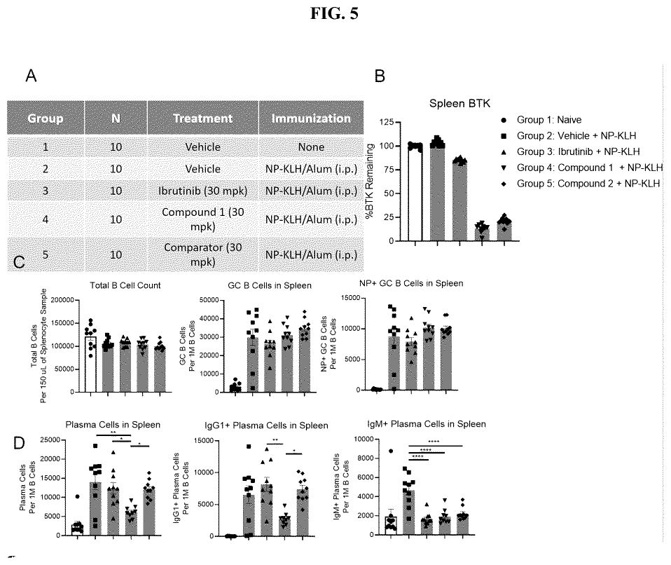

FIG. 6 provides clinical arthritis scores for compound 1, comparator compounds, and a comparator antibody (FIG. 6A), plasma cell counts in spleens for compound 1, comparator compounds, and a comparator antibody (FIG. 6B); and plasma cell counts in bone marrow for compound 1, comparator compounds, and a comparator antibody (FIG. 6C).

FIG. 7 provides compound 1 exposure in mouse cerebrospinal fluid over time following a single dose.

FIG. 8 provides tumor burden (FIG. 8A) and BTK degradation (FIG. 8B) in a mouse brain tumor model.

### DETAILED DESCRIPTION

Provided herein are methods of using bifunctional compounds that induce the proteolytic degradation of Bruton's tyrosine kinase (BTK) via a ubiquitin proteolysis pathway.

As used herein, the following definitions shall apply unless otherwise indicated.

### Definitions

For purposes herein, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As described herein, “IMiD” activity indicates Immunomodulatory imide Drug activity. In certain embodiments, IMiD activity is relative to an IMiD compound. In certain embodiments, the IMiD compound is selected from the group consisting of thalidomide, lenalidomide, pomalidomide, iberdomide, and apremilast. In certain embodiments, IMiD activity is measured with downregulation of an IMiD target. In certain embodiments, the target is Aiolos. In certain embodiments, the target is Ikaros. In certain embodiments, “Low IMiD activity” indicates a maximum degradation of Aiolos of less than 50%, 40%, 30%, 25%, 20%, 15%, or 10% under physiological conditions. In certain embodiments, “Low IMiD activity” indicates a maximum degradation of Ikaros of less than 50%, 40%, 30%, 25%, 20%, 15%, or 10% under physiological conditions. Exemplary assays for Aiolos degradation are provided in the Examples herein.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds herein optionally may be substituted with one or more substituents, such as those illustrated generally herein, or as exemplified by particular classes, subclasses, and species of the description.

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aryl, heteroaryl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonyl amino.” These terms when used alone or in connection with another group refer to an amido group such as —N(R^{X})—C(O)—R^{Y} or —C(O)—N(R^{X})₂, when used terminally, and or —N(R^{X})—C(O)— when used internally, wherein R^{X} and R can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl (e.g., mono-, di- (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkyl sulfonyl)amino)aryl or ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkyl sulfonyl)amino)alkyl)aryl; ((heterocycloaliphatic)carbonyl)aryl; ((alkyl sulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9-, 10-, or 11-) membered structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryl s.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonyl amino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused, bridged, or spiro) (e.g., 5- to 10-membered mono- or bicyclic) saturated ring structure, in which one or more of the ring atoms is a heteroatom (e.g., nitrogen (N), oxygen (O), sulfur (S), or combinations thereof). Non-limiting examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholinyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl A monocyclic heterocycloalkyl group can be fused with a phenyl moiety to form structures, such as tetrahydroisoquinoline, that would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes benzo fused with one or two 4- to 8-membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, 2H-pyranyl, 4H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkyl sulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkyl sulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkyl sulfonyl)amino)alkyl)heteroaryl; ((alkyl sulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C14 alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein.

As used herein, an “alkoxy” group refers to an alkyl-O— group where “alkyl” has been defined previously herein.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)— or —C(O)O— when used as an internal group.

As used herein, an ester refers to —COOR^{X} when used as a terminal group; or —COOR^{X}— when used as an internal group, wherein R^{X} has been defined above.

As used herein, a formate refers to —OC(O)H.

As used herein, an acetate refers to —OC(O)R^{X}, wherein R^{X} has been defined above.

As used herein, a “haloaliphatic” group refers to an aliphatic group substituted with one to three halogen. For instance, the term haloalkyl includes the group —CF₃.

As used herein, a “mercapto” or “sulfhydryl” group refers to —SH.

As used herein, a “sulfa” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R_{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}- or —NR’—S(O)₂— when used internally, wherein R^{X}, R_{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))—S(O)—, cycloalkyl-S(O)—, heterocycloaliphatic-S(O)—, heteroaryl-S(O)—, and/or the like.

As used herein, a “sulfonyl” group refers to S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, and/or the like.

As used herein, a “sulfoxy” group refers to —O—S(O)—R^{X} or —S(O)—O—R^{X}, when used terminally and —O—S(O)— or —S(O)—O— when used internally, where R^{X} has been defined above.

As used herein, a “halogen” or “halo” group refers to fluorine (F), chlorine (Cl), bromine (Br), or iodine (I).

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy, aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} each when used terminally and —NR^{X}—CO—NR^{Y}— or —NR^{X}—CS—NR^{Y}— each when used internally, wherein R^{X}, R^{Y}, and R^{Z} have been defined above.

As used herein, a “guanidine” group refers to the structure —N═C(N(R^{X}R^{Y})N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl (i.e., R^{X}O(O)C-alkyl) is an example of a carboxy group used terminally. A group is internal when the group is present in the middle of or within the termini of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ-, where each Q is independently a hydrogen (H or —H) or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used herein interchangeably with the phrase “substituted or unsubstituted.” As described herein, compounds herein can optionally be substituted with one or more substituents, such as are illustrated generally above, or as exemplified by particular classes, subclasses, and species of the description. As described herein, the variables R, R¹, R², L, Y, and Z, and other variables contained in Formula A-X or I-IV described herein encompass specific groups, such as alkyl and aryl. Unless otherwise noted, each of the specific groups for the variables R, R¹⁰, R^{A}, R¹, R², L, L¹, D, W, E, V, G, Y, and Z, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

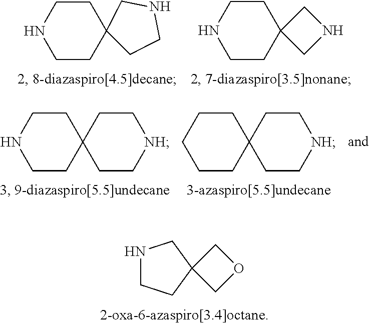

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

As used herein, the term “about” means within ±10% of a value. For example, a dose that is about 100 mg/kg provides that the does can 90 mg/kg to 110 mg/kg. By way of further example, an amount of an additional therapeutic agent ranging from about 50% to about 100% provides that the amount of additional therapeutic agent ranges from 45-55% to 90-110%. A person of skill in the art will appreciate the scope and application of the term “about” when used to describe other values disclosed herein.

Unless otherwise stated, structures depicted herein also are meant to include all isomeric (e.g., enantiomeric, diastereomeric, and geometric (or conformational)) forms of the structure; for example, the (R)- and (S)- configurations for each asymmetric center, (Z)— and (E)- double bond isomers, and (Z)— and (E)-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]−[(S)-compound])/([(R)-compound]+[(S)-compound])×100, where the (R)-compound>(S)-compound; or % ee=([(S)-compound]−[(R)-compound])/([(S)-compound]+[(R)-compound])×100, where the (S)-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de greater than zero. For example, in certain embodiments, the compounds or inhibitors described herein have an ee, de, % ee, or % de of ten. In certain embodiments, the compounds or inhibitors described herein have an ee, de, % ee, or % de of twenty-five. In certain embodiments, the compounds or inhibitors described herein have an ee, de, % ee, or de of fifty. In certain embodiments, the compounds or inhibitors described herein have an ee, de, % ee, or % de of seventy-five.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or % de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or % de is one. In one embodiment of a compound or inhibitor described herein, the ee, de, % ee, or % de is two. In one embodiment of a compound or inhibitor described herein, the ee, de, % ee, or % de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two. In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or % de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee, de, % ee, or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or % de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, any of compounds 1-22, as described in the Examples and/or Biological Examples have an ee, de, % ee, or % de as described within this paragraph. Unless otherwise stated, all tautomeric forms of the compounds of the description are within the scope of the description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “& 1” means that a compound including the “& 1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 11.0.1, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### Uses of the Compounds and Compositions

Bifunctional compounds that degrade BTK have been previously described, for example in PCT/US2019/56112, filed Oct. 14, 2019, published as WO 2020/081450, Apr. 23, 2020, and PCT/US2020/063176, filed Dec. 3, 2020, published as WO 2021/113557, Jun. 10, 2021, each of which is incorporated by reference in its entirety. Because many of these BTK degraders also have IMiD activity, these compounds have increased toxicity from IMiD activity which limits their potential uses where IMiD activity is not expected to provide an additional therapeutic effect. In contrast, in some embodiments, the bifunctional compounds described herein are useful for degrading BTK in biological samples or in patients with little or no IMiD activity. Thus, an embodiment of this disclosure provides a method of treating a BTK-mediated disease or disorder. As used herein, the term BTK-mediated disease or disorder” means any disease, disorder, or other deleterious condition in which a BTK is known to play a role. In some instances, a BTK-mediated disease or disorder is a proliferative disorder or an autoimmune disorder or an inflammatory disorder. Examples of proliferative disorders include cancer.

The IMiD activity of the compound can be measured by any technique deemed suitable by the person of skill. In certain embodiments, IMiD activity is measured as Aiolos degradation. In certain embodiments, IMiD activity is measured as Ikaros degradation. In certain embodiments, IMiD activity is measured as IL2 activation. In certain embodiments, IMiD activity is measured as any combination of these. In certain embodiments, IMiD activity is measured in vivo. In certain embodiments, IMiD activity is measured in vitro, for instance in cell based assays.

In certain embodiments, IMiD activity of the compound is less than 30% of the IMiD activity of a comparator compound. In certain embodiments, IMiD activity of the compound is less than 25% of the IMiD activity of a comparator compound. In certain embodiments, IMiD activity of the compound is less than 20% of the IMiD activity of a comparator compound. In certain embodiments, IMiD activity of the compound is less than 15% of the IMiD activity of a comparator compound. In certain embodiments, IMiD activity of the compound is less than 10% of the IMiD activity of a comparator compound. In certain embodiments, IMiD activity of the compound is less than 5% of the IMiD activity of a comparator compound. In certain embodiments, the comparator compound is thalidomide, lenalidomide, or pomalidomide. In certain embodiments, activity is measured as IC₅₀ or EC₅₀ or DC₅₀. In certain embodiments, activity is measured as Dₘₐₓ. In certain embodiments, activity is measured by Western blot.

In the methods, the compounds comprise a moiety capable of specifically binding BTK and further comprise a moiety capable of recruiting an ubiquitin ligase to degrade the BTK. Particular compounds are described herein. The compounds can be administered in any form, including pharmaceutically acceptable salts and pharmaceutical compositions.

Due to the low IMiD activity, the compounds described herein can yield less toxicity compared to other BTK degrading compounds. The lower toxicity can provide for higher doses of the compounds, increased duration of therapy, increased frequency of therapy, or any combination thereof.

In certain embodiments, the compounds are administered chronically. “Chronic administration” and “chronically” refer to an administration that continues on a schedule for over 14 days. In certain embodiments, the compound is administered for at least 15 days, at least 20 days, at least two weeks, at least three weeks, at least four weeks, at least one month, at least two months, at least three months, at least six months, at least one year, or longer.

In the dosing schedule, the doses can be administered on consecutive days or cyclically, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the frequency of chronically administrating is daily. In certain embodiments, the frequency of chronically administering is twice a day. In certain embodiments, the frequency of chronically administering is thrice a day. In certain embodiments, the frequency of chronically administering is frice a day. In certain embodiments, the frequency of chronically administering is once a week. In certain embodiments, the frequency of chronically administering is twice a week.

In certain embodiments, the dose(s) are administered for a period of time with a first interval between dose(s), and then the dose(s) are re-administered for a period of time following the first interval between dose(s), wherein this dosing regimen can be repeated (i.e., cyclically or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered in any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg.

In certain embodiments, the dose is selected from the group consisting of 10-100 mg/kg, 20-30 mg/kg, and 45-55 mg/kg. In certain embodiments, the dose is 10-100 mg/kg. In certain embodiments, the dose is 20-30 mg/kg. In certain embodiments, the dose is 45-55 mg/kg. In certain embodiments, the dose is about 10 mg/kg. In certain embodiments, the dose is about 100 mg/kg. In certain embodiments, the dose is about 20 mg/kg. In certain embodiments, the dose is about 30 mg/kg. In certain embodiments, the dose is about 45 mg/kg. In certain embodiments, the dose is about 55 mg/kg. In certain embodiments, the dose is 10 mg/kg. In certain embodiments, the dose is 100 mg/kg. In certain embodiments, the dose is 20 mg/kg. In certain embodiments, the dose is 30 mg/kg. In certain embodiments, the dose is 45 mg/kg. In certain embodiments, the dose is 55 mg/kg.

In certain embodiments, the dose is 100-600 mg/kg. In certain embodiments, the dose is 200-600 mg/kg. In certain embodiments, the dose is 250-600 mg/kg. In certain embodiments, the dose is 300-600 mg/kg. In certain embodiments, the dose is selected from the group consisting of 50 mg/kg, 100 mg/kg, 200 mg/kg, 300 mg/kg, 450 mg/kg, 600 mg/kg, 800 mg/kg, and 1000 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The compound can be administered by any route of administration deemed suitable by the practitioner of skill. In certain embodiments, the dose is administered orally. Formulations and techniques for administration are described in detail below.

In certain embodiments, the methods are for the treatment or prevention of a cancer, autoimmune condition, or inflammatory condition.

In one aspect, provided herein are methods of treating or preventing cancer in a subject in need thereof. In certain embodiments, the methods comprise the step of orally administering to the subject an amount of a bifunctional compound capable of inducing proteolytic degradation of Bruton's tyrosine kinase. In certain embodiments, the amount is effective to treat or prevent the cancer.

In certain embodiments, the cancer is any cancer described below. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation.

In certain embodiments, the subject has a mutant Bruton's tyrosine kinase. In certain embodiments, the subject has a C481 mutant Bruton's tyrosine kinase. In certain embodiments, the subject has a C481 S mutant Bruton's tyrosine kinase. In certain embodiments, the cancer is resistant to ibrutinib. Those of skill will recognize that certain ibrutinib-resistant cancers express a C481 mutant Bruton's tyrosine kinase, for instance C481 S Bruton's tyrosine kinase. For example, in certain embodiments, the subject has a C481 mutant Bruton's tyrosine kinase and the cancer is chronic lymphocytic leukemia (CLL).

In another aspect, provided herein are methods of degrading Bruton's tyrosine kinase in a subject in need thereof. The methods comprise the step of orally administering to the subject an amount of a bifunctional compound capable of inducing proteolytic degradation of Bruton's tyrosine kinase. In certain embodiments, the amount is effective to degrade Bruton's tyrosine kinase in the subject. The Bruton's tyrosine kinase can be expressed in any cells or tissues of the subject. In certain embodiments, the Bruton's tyrosine kinase is expressed in splenocytes. In certain embodiments, the Bruton's tyrosine kinase is expressed in peripheral blood mononuclear cells.

In certain embodiments, the Bruton's tyrosine kinase is a mutant form. In certain embodiments, Bruton's tyrosine kinase comprises a C481 mutation. In certain embodiments, the Bruton's tyrosine kinase comprises a C481S mutation. In certain embodiments, the Bruton's tyrosine kinase is resistant to ibrutinib.

In another aspect, provided herein are methods of preventing B cell activation in a subject in need thereof. The methods comprise the step of orally administering to the subject an amount of a bifunctional compound capable of inducing proteolytic degradation of Bruton's tyrosine kinase. In certain embodiments, the amount is effective to prevent B cell activation. In certain embodiments, the B cell expresses CD69. In certain embodiments, the B cell expresses CD86. In certain embodiments, the B cell expresses CD69 and CD86.

In another aspect, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase. In certain embodiments, the amount is effective to degrade the mutant Bruton's tyrosine kinase. In certain embodiments, the mutant Bruton's tyrosine kinase is a C481 mutant. In certain embodiments, the mutant Bruton's tyrosine kinase is a C481 S mutant.

In certain embodiments, term “cancer” includes, but is not limited to, the following cancers: epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenström's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metatstaic melanoma.

In certain embodiments, the cancer is brain cancer. In certain embodiments, the cancer is acoustic neuroma. In certain embodiments, the cancer is astrocytoma. In certain embodiments, the cancer is pilocytic astrocytoma. In certain embodiments, the cancer is juvenile pilocytic astrocytoma. In certain embodiments, the cancer is low-grade astrocytoma. In certain embodiments, the cancer is anaplastic astrocytoma. In certain embodiments, the cancer is glioblastoma. In certain embodiments, the cancer is chordoma. In certain embodiments, the cancer is CNS lymphoma. In certain embodiments, the cancer is craniopharyngioma. In certain embodiments, the cancer is glioma. In certain embodiments, the cancer is brain stem glioma. In certain embodiments, the cancer is ependymoma. In certain embodiments, the cancer is mixed glioma. In certain embodiments, the cancer is optic nerve glioma. In certain embodiments, the cancer is subependymoma. In certain embodiments, the cancer is medulloblastoma. In certain embodiments, the cancer is meningioma. In certain embodiments, the cancer is metastatic brain tumor. In certain embodiments, the cancer is oligodendroglioma. In certain embodiments, the cancer is pituitary tumor. In certain embodiments, the cancer is primitive neuroectodermal (PNET). In certain embodiments, the cancer is rhabdoid tumor. In certain embodiments, the cancer is schwannoma.

In certain embodiments, the disease is selected from the group consisting of Waldenstrom's macroglobulinemia, marginal zone lymphoma, mantle cell lymphoma, primary central nervous system lymphoma, and chronic lymphocytic leukemia. In certain embodiments, the disease is Waldenstrom's macroglobulinemia. In certain embodiments, the disease is marginal zone lymphoma. In certain embodiments, the disease is mantle cell lymphoma. In certain embodiments, the disease is primary central nervous system lymphoma. In certain embodiments, the disease is chronic lymphocytic leukemia.

Examples of autoimmune disorders include uticaria, graft-versus-host disease (GVHD), acute graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive Arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)). In certain embodiments, the autoimmune disorder is warm autoimmune hemolytic anemia (wAIHA). In certain embodiments, the autoimmune disorder is systemic sclerosis. In certain embodiments, the autoimmune disorder is systemic sclerosis membranous nephropathy.

Examples of inflammatory disorders include encephalitis, myelitis, meningitis, arachnoiditis, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, soft tissue, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

In certain embodiments, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase. In certain embodiments, the amount of a bifunctional compound capable of inducing proteolytic degradation of Bruton's tyrosine kinase is the amount effective to degrade the mutant Bruton's tyrosine kinase. In certain embodiments, the mutant Bruton's tyrosine kinase is a C481 mutant. In certain embodiments, the mutant Bruton's tyrosine kinase is a C481 S mutant. The contacting can be in vitro or in vivo. In certain embodiments, the contacting is in vitro. In certain embodiments, the contacting is in vivo. In certain embodiments, the contacting is in a subject in need thereof.

### Compounds

The methods provided herein comprise administration of a compound. The compound can be any compound described herein. In certain embodiments, the compound comprises at least two moieties. One moiety is capable of specifically binding Bruton's tyrosine kinase (BTK). The other moiety is capable of recruiting an ubiquitin ligase to degrade the BTK. In certain embodiments, the ubiquitin ligase is an E3 ligase. In certain embodiments, the ubiquitin ligase is cereblon (CRBN) or comprises cereblon as a component.

In the methods, the compound can be a compound of Formula (A1)

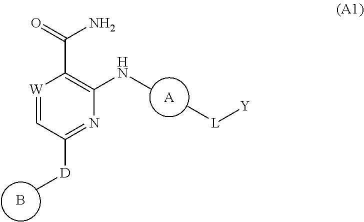

or a pharmaceutically acceptable salt thereof, wherein W is CH or N; D is a bond or a linker; Ring A is aryl or heteroaryl; Ring B is aryl or heteroaryl; L is a bond or a linker; and Y is a moiety capable of binding an ubiquitin ligase.

In the methods, the compound can be a compound of Formula (A)

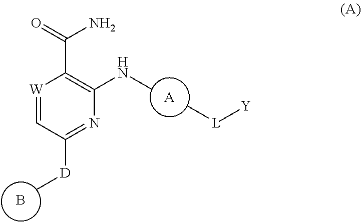

or a pharmaceutically acceptable salt thereof, wherein W is CH or N; D is a bond or —NH—; Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S, wherein Ring A is optionally and independently substituted with up to three substituents selected from halo, —CN, —COOH, NH₂, and optionally substituted C₁₋₆ alkyl; Ring B is a phenyl, a 5-6 membered heteroaryl, a 4-6 membered heterocycloalkyl, or a 8-10 membered (e.g., 8-9 membered or 9-10 membered) spiro bicyclic heterocycle, wherein Ring B is optionally substituted, and wherein the heteroaryl and heterocycloalkyl of Ring B has one to three heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S; X⁵ is a bond, —C₁₋₄ alkyl-, —N(R)—, —O—, —C(O)—, or —C(O)—N(R)—; each R is independently —H or —C₁₋₃ alkyl (e.g., methyl, ethyl, propyl, or iso-propyl); and each of m, n, and p is independently an integer from one to three (e.g., one, two, or three); and Y is

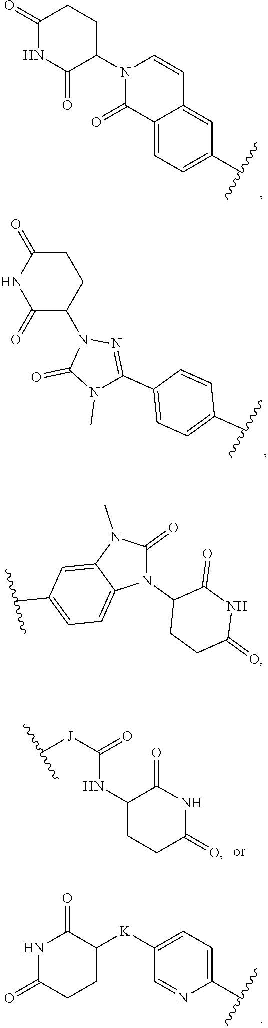

wherein each J is independently aryl or heteroaryl; and each K is independently absent, —CH₂—, —NH—, —NMe—, or —O—; wherein each C₁₋₄ alkyl is optionally and independently substituted with up to three instances of halo, —CN, —COOH, —COONH₂, —NH₂, or —CF₃.

With the exception of the moieties of group R, all moieties of the linking group L as defined in the compound of Formula (A) are bivalent moieties unless otherwise specified. For example, any alkyl (e.g., n-propyl, n-butyl, n-hexyl, and the like), aryl (e.g., phenyl), cycloalkyl (e.g., cyclopropyl, cyclohexyl, and the like), heteroaryl, heterocycloalkyl (e.g., piperidine, piperazine, and the like) that is present in L is bivalent unless otherwise specified.

In some embodiments, Ring B is an optionally substituted 5-6 membered heterocycloalkyl having one to two nitrogen atoms. For example, Ring B is piperidine-yl, piperizine-yl, or pyrrolidine-yl, any of which is optionally substituted.

In some embodiments, Ring B is an optionally substituted 5-6 membered heteroaryl having one to two heteroatoms independently selected from N and S. For example, Ring B is pyridine-yl, pyrazine-yl, or pyrimidine, any of which is optionally substituted.

In some embodiments, Ring B is

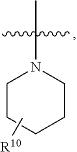

wherein R¹⁰ is halo, —H, —C₁₋₅ alkyl (e.g., —C₁₋₃ alkyl), 3-6 membered cycloalkyl, 5-6 membered heterocycloalkyl, —CN, —OH, —CF₃, —CH₂OH, —CH₂CH₂OH, —C(O)OH,

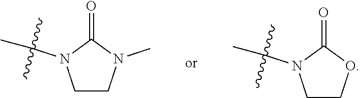

In some embodiments, Ring B is

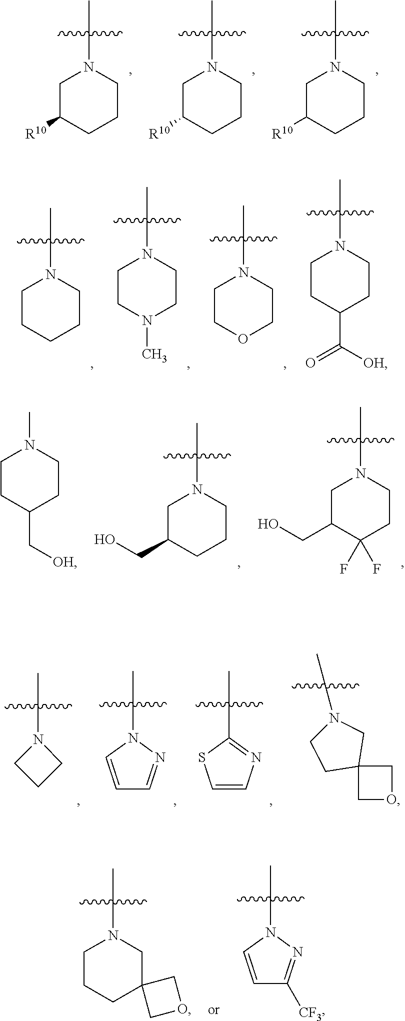

wherein R¹⁰ is

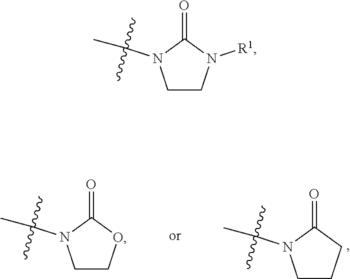

and wherein R¹ is a C₁₋₄ alkyl group. For example, Ring B is

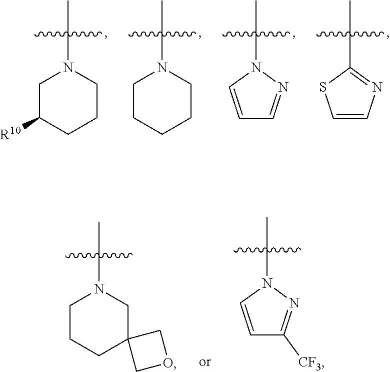

wherein R¹⁰ is

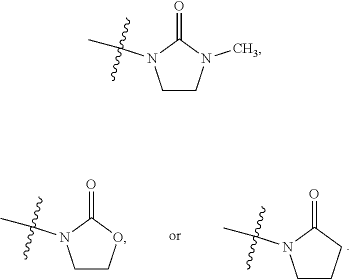 [SMILES: *N1CCN(C)C1=O ; *N1CCOC1=O ; *N1CCCC1=O]

And in some instances, Ring B is

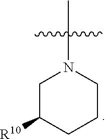

In other instances, R¹⁰ is

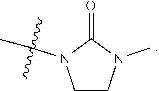 [SMILES: *N1CCN(C)C1=O]

In some embodiments, Ring A is

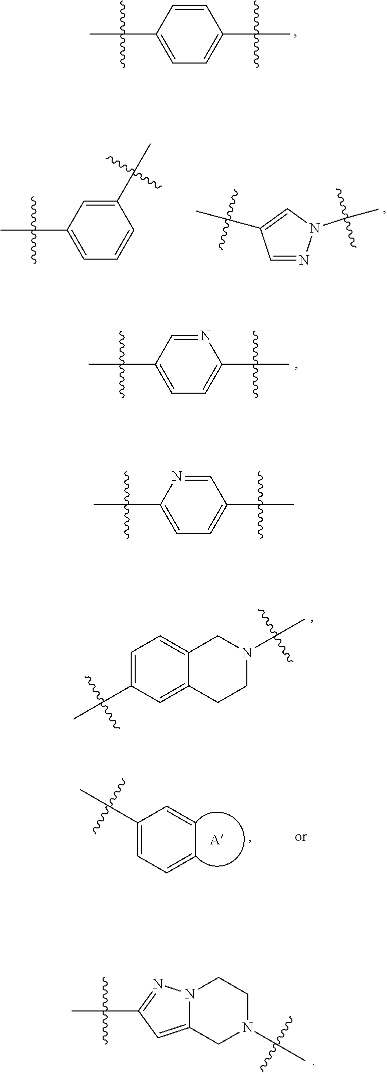

wherein Ring A′ together with the phenyl ring to which Ring A′ is fused form a 9-10 membered bicyclic aryl or a 9-10 membered bicyclic heteroaryl wherein the bicyclic heteroaryl (i.e., the bicyclic heteroaryl including Ring A′) has one to three heteroatoms independently selected from N, O, or S. For example, Ring A is

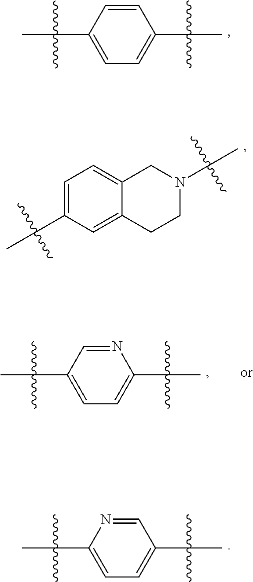 [SMILES: *c1ccc(*)cc1 ; *c1ccc2c(c1)CCN(*)C2 ; *c1ccc(*)nc1 ; *c1ccc(*)nc1]

In some embodiments, at least one of X¹, X², and X⁵ is —N(R)—, —C(O)—N(R)—, or —CH₂—.

In some embodiments, X¹ is —C(O)—N(R)—.

In some embodiments, X² is —(O—CH₂—CH₂)ₙ—, —(CH₂—CH₂—O)ₙ—, or —C₁₋₅ alkyl-.

In some embodiments, X³ is a bond, —C≡C—, —C₁₋₄ alkyl-, or —N(R)—.

In some embodiments, X⁴ is a bond, —CH₂—, or —N(R)—.

In some embodiments, X⁵ is a bond.

In some embodiments, X¹ is —(O—CH₂—CH₂—CH₂)ₘ—, m is one, and X² is —C(O)—N(R)—.

In some embodiments, X¹ is —CH₂—, —C(O)—,

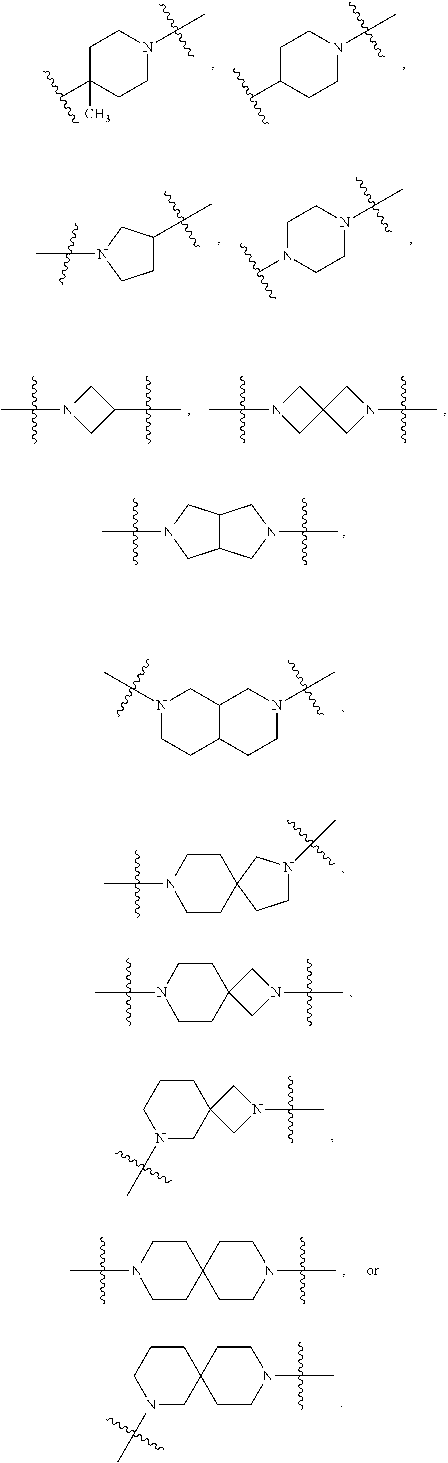

In some embodiments, X² is a bond, —C(O)—, —C₁₋₅ alkyl-,

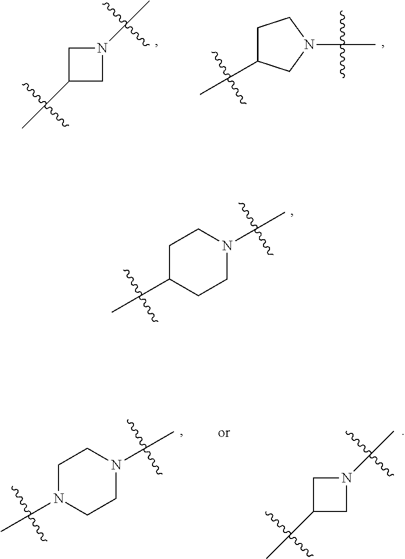 [SMILES: *C1CN(*)C1 ; *C1CCN(*)C1 ; *C1CCN(*)CC1 ; *N1CCN(*)CC1 ; *C1CN(*)C1]

In some embodiments, X³ is bond, —C₁₋₄ alkyl-, 4-6 membered cycloalkyl, or —N(R)—.

In some embodiments, X³ is a bond, C₁₋₄ alkyl NH—,

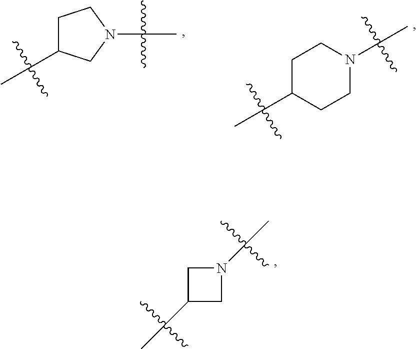 [SMILES: *C1CCN(*)C1 ; *C1CCN(*)CC1 ; *C1CN(*)C1]

### or —C≡C—.

In some embodiments, X⁴ is a bond,

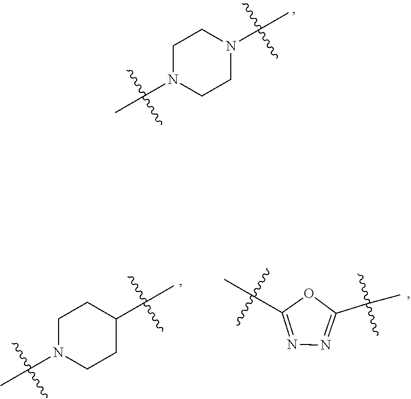 [SMILES: *N1CCN(*)CC1 ; *C1CCN(*)CC1 ; *c1nnc(*)o1]

—C₁₋₄ alkyl-, —CH₂—CH₂—N(R)—, or —N(R)—.

In some embodiments, X⁵ is a bond, —C₁₋₄ alkyl-, —N(R)—, or —C(O)—N(R)—.

In some embodiments, L is

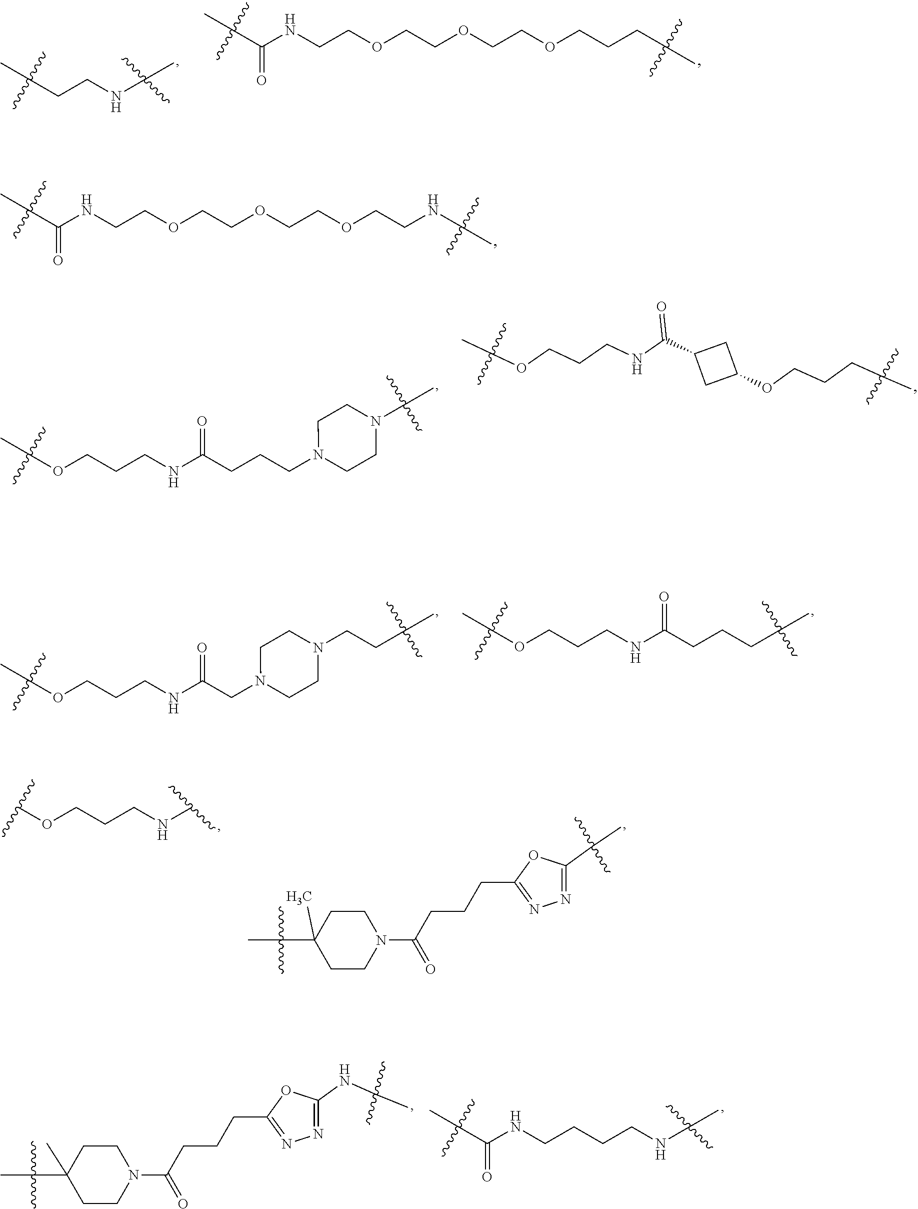

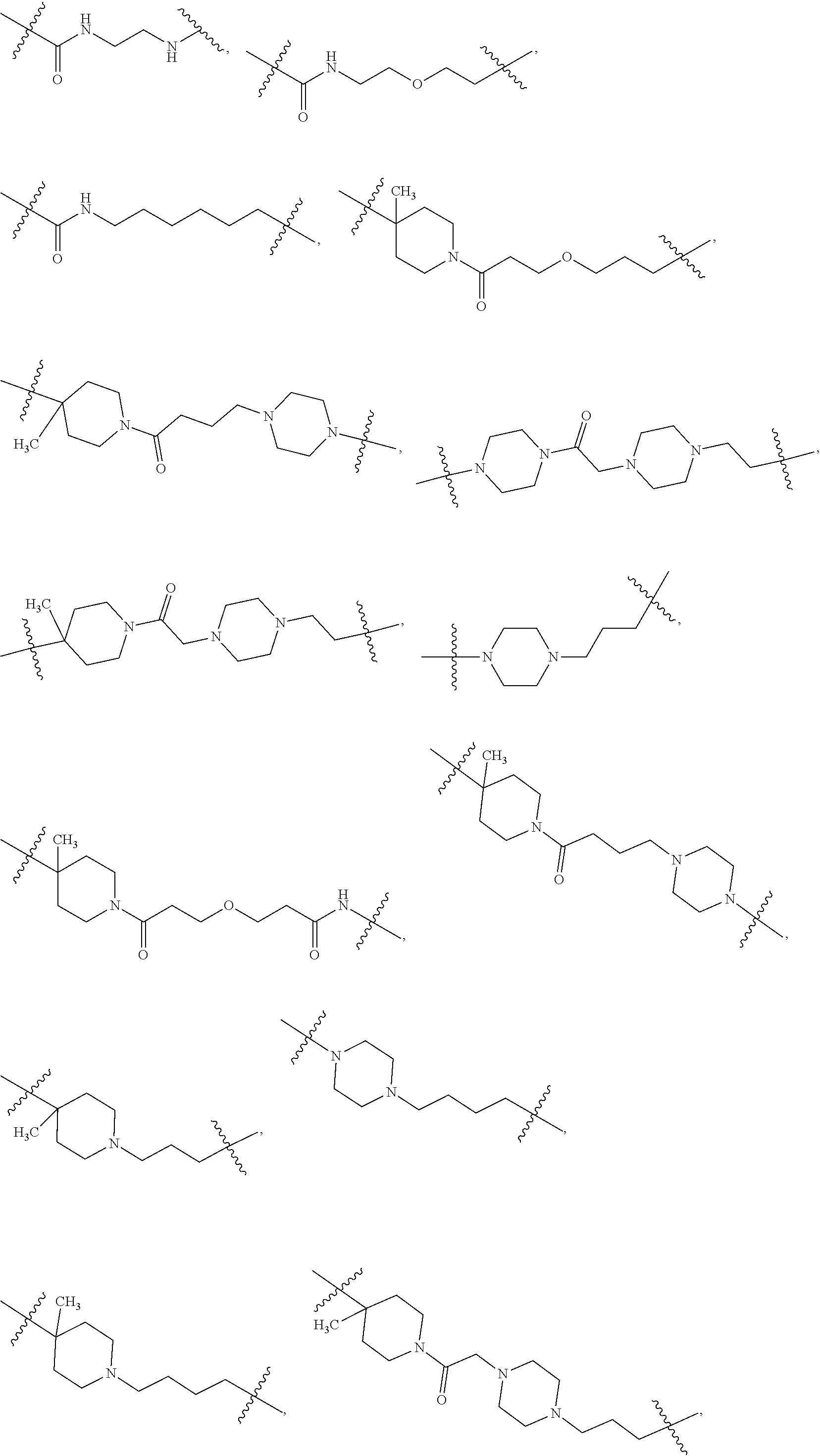

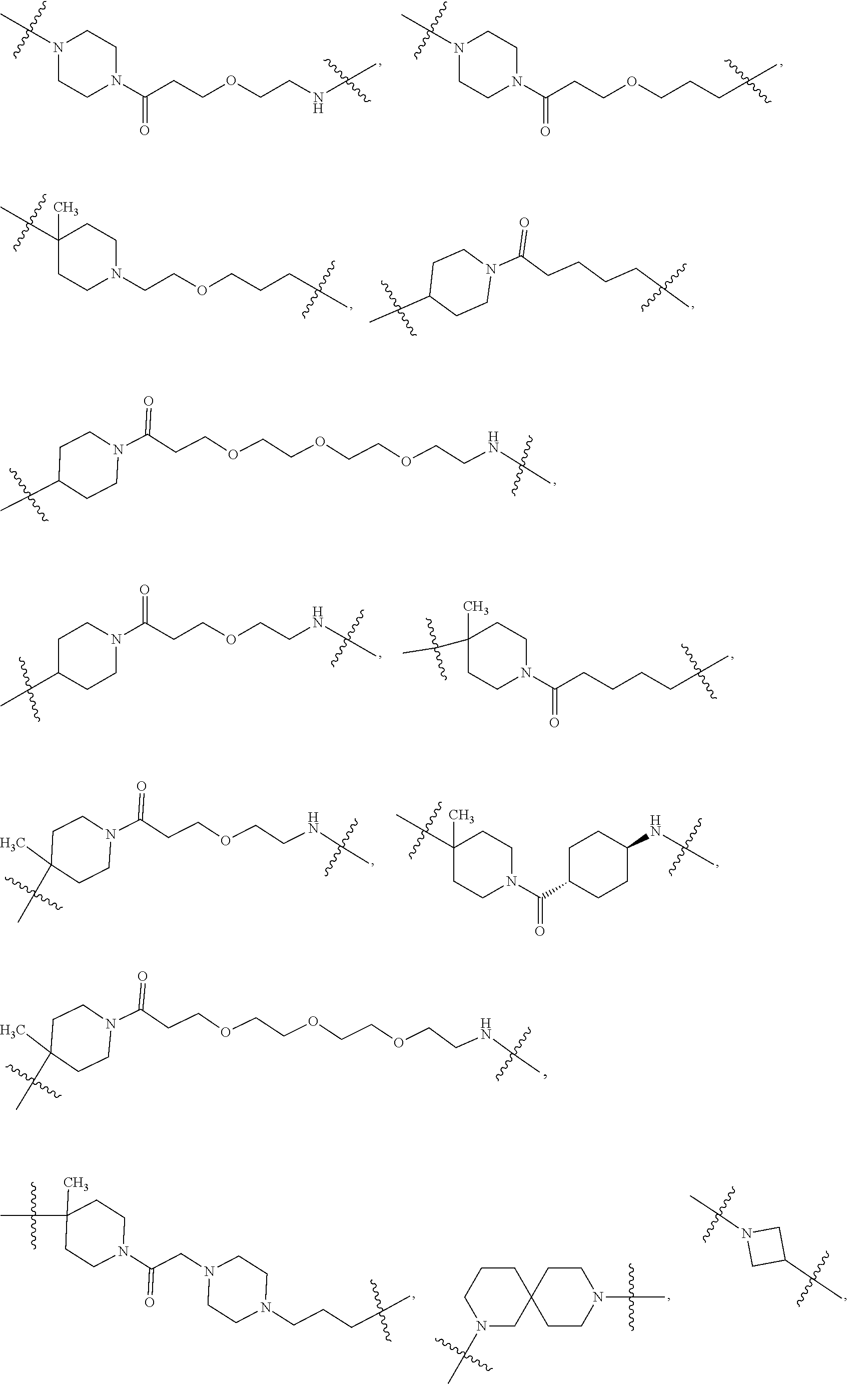

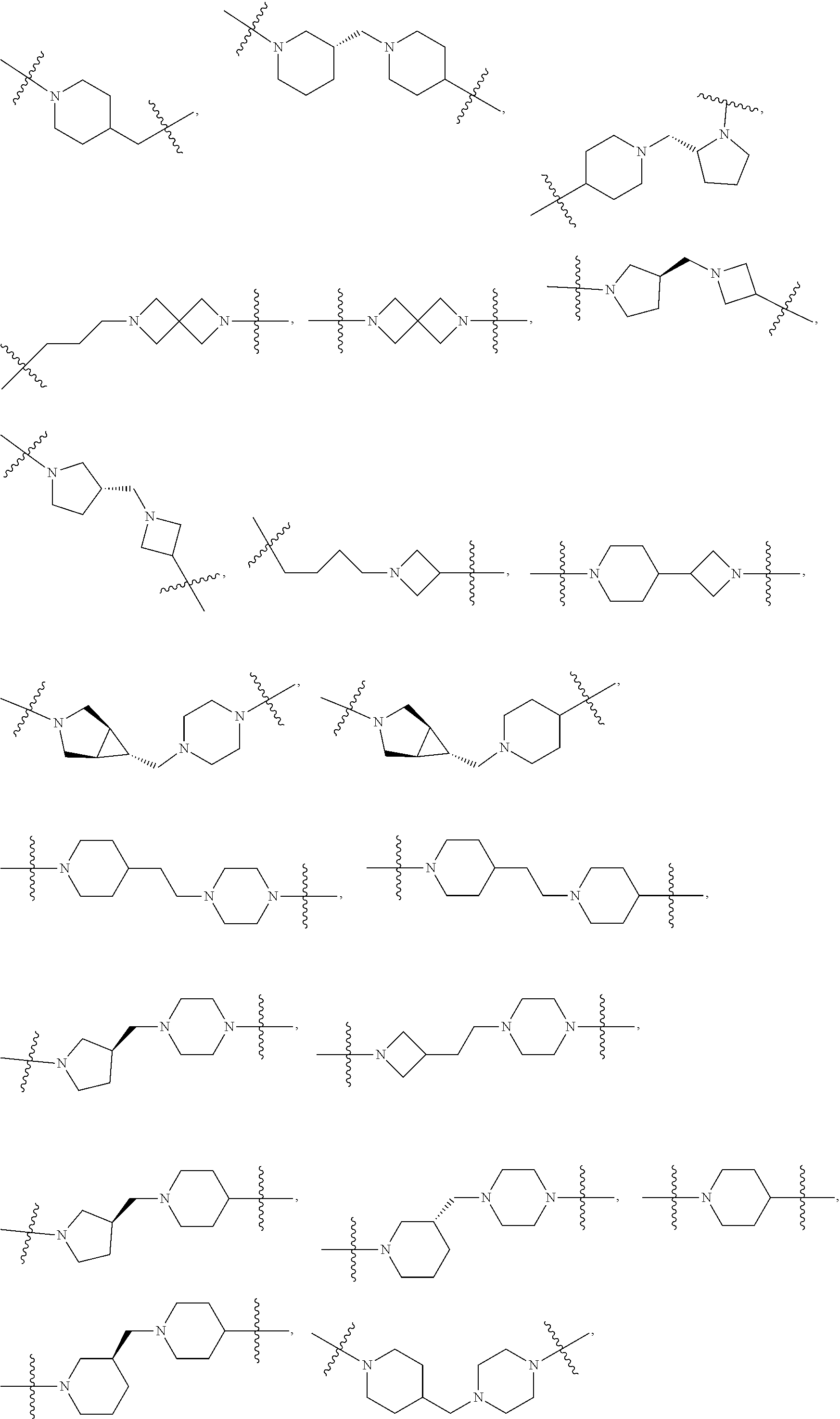

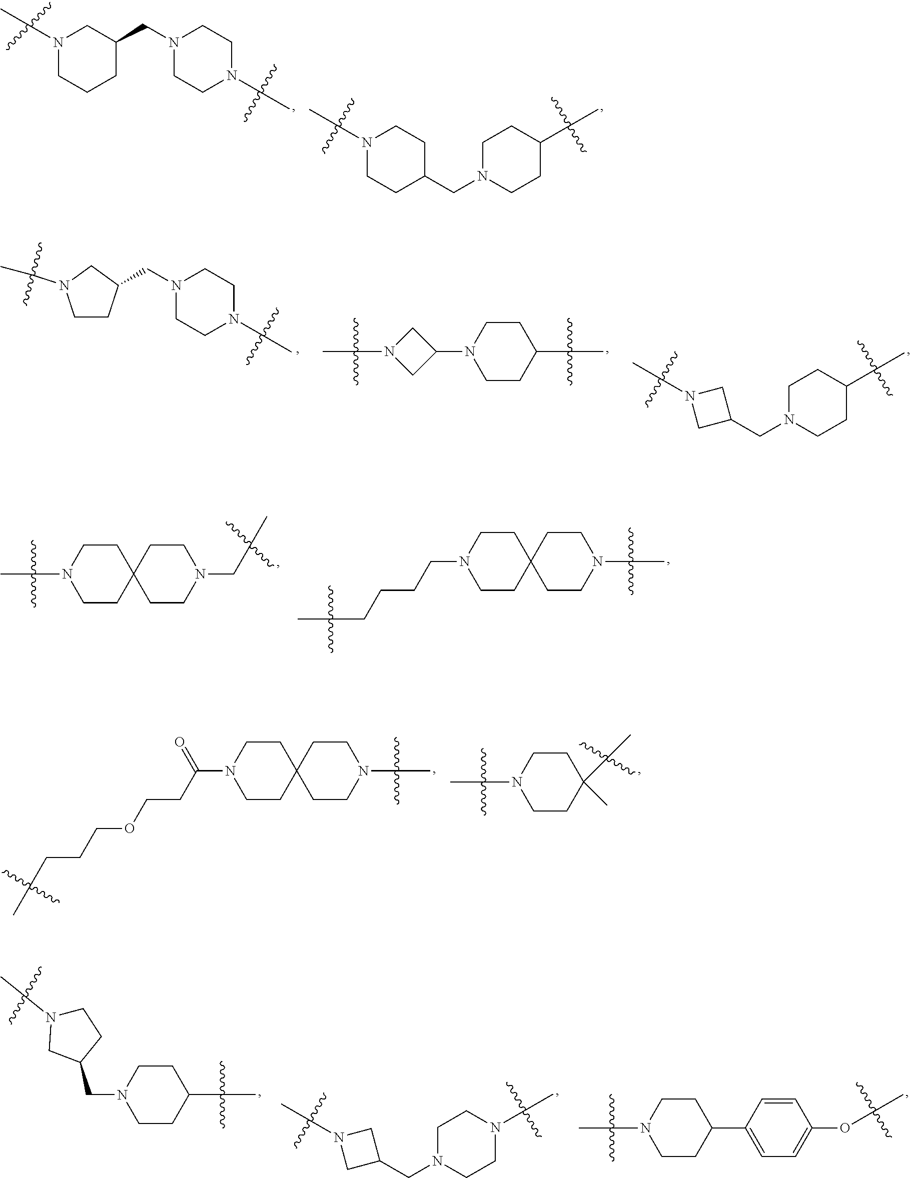

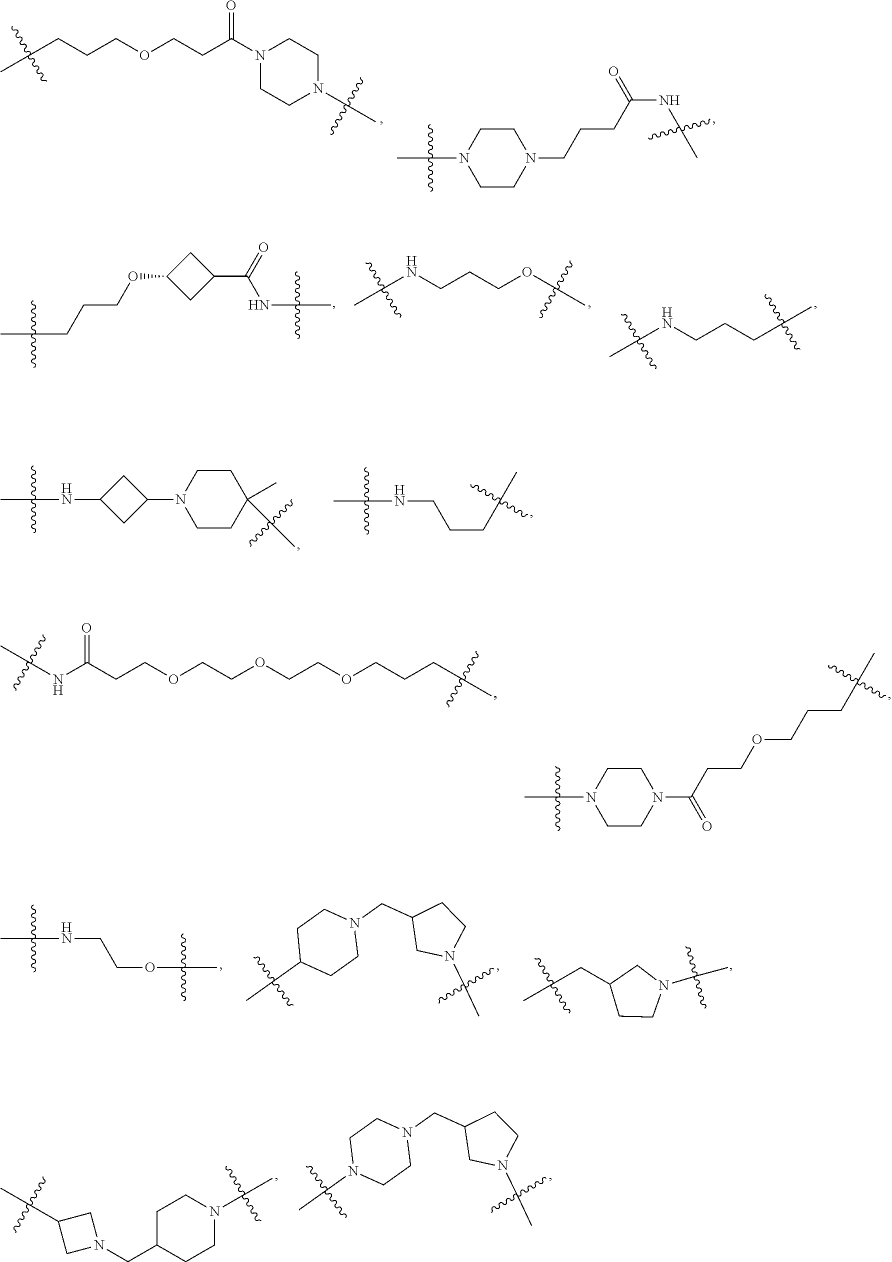

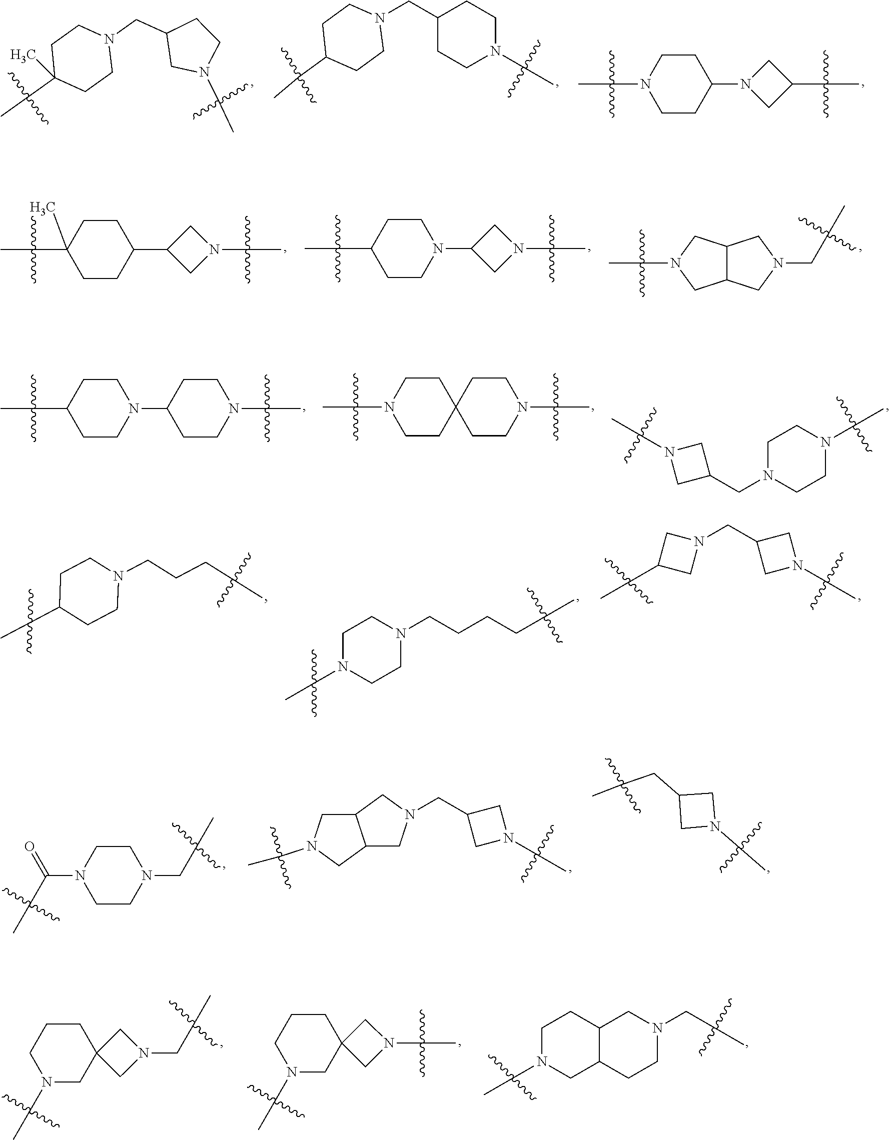

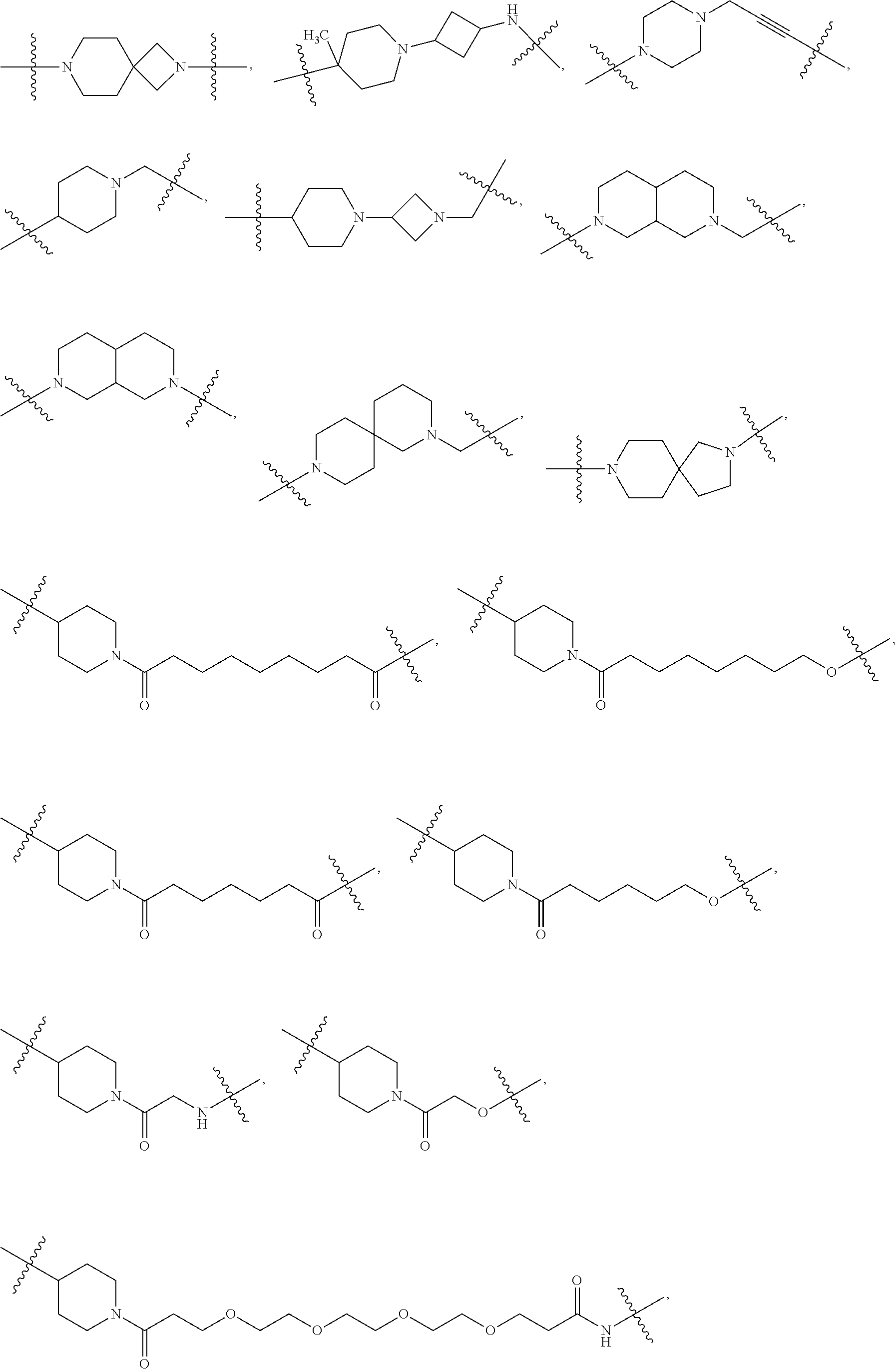

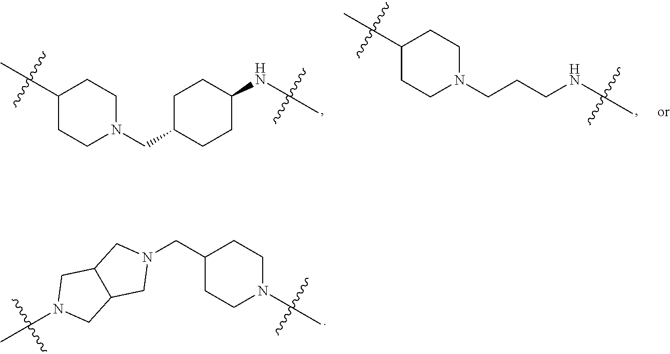 [SMILES: *N[C@H]1CC[C@H](CN2CCC(*)CC2)CC1 ; *NCCCN1CCC(*)CC1 ; *N1CCC(CN2CC3CN(*)CC3C2)CC1]

In some embodiments, Y is

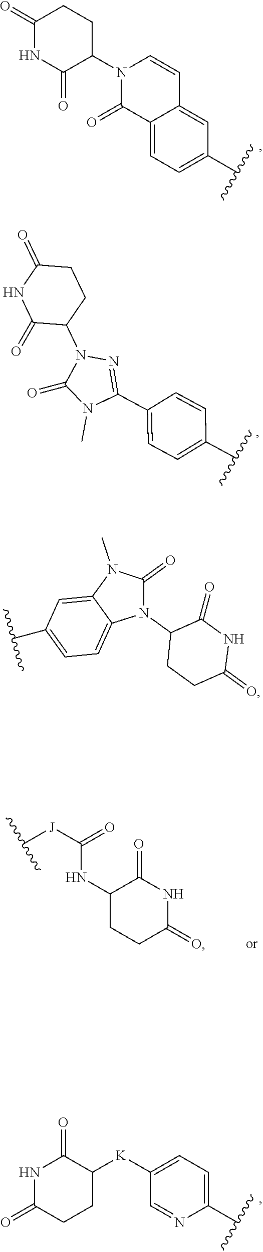

wherein each J is independently aryl or heteroaryl, and each K is independently absent, —CH₂—, —NH—, —NMe—, or —O—.

In some embodiments, Y is

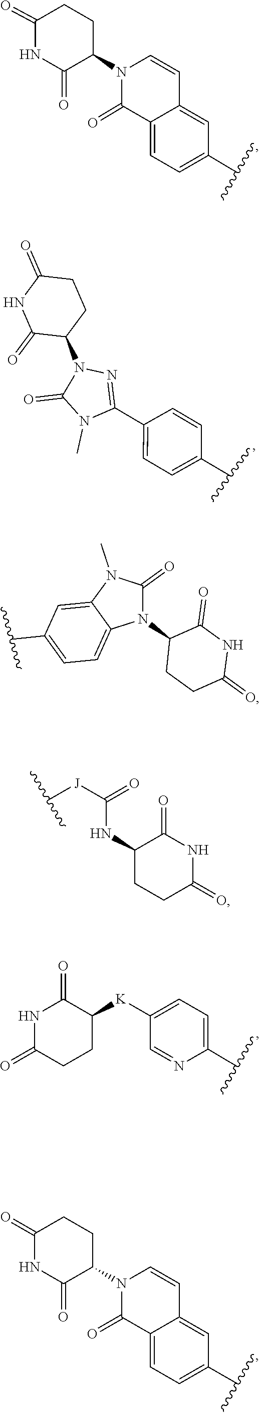

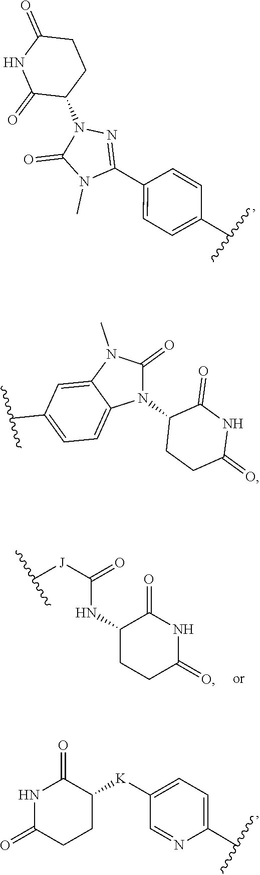

wherein each J is independently aryl or heteroaryl, and each K is independently absent, —CH₂—NH—, —NMe—, or —O—.

This disclosure also provides a compound of Formula (B)

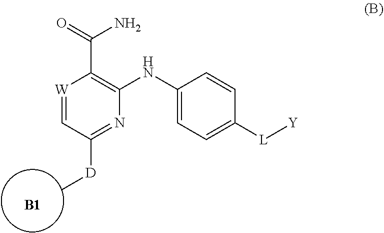

or a pharmaceutically acceptable salt thereof, wherein W is CH or N; D is a bond or —NH—; Ring B1 is a 4-6 membered, fully saturated, partially unsaturated, or fully unsaturated monocyclic heterocycle or a 8-10 membered, fully saturated, spiro bicyclic heterocycle, wherein Ring B1 has one to three heteroatoms independently selected from N, O, or S, and is optionally substituted with one to three groups selected from halo, —CH₃, —CF₃, —C(O)OH, —CH₂OH, or a 5-membered heterocycloalkyl optionally substituted with oxo and having one to two heteroatoms independently selected from N or O; L is —X¹—X²—X³—; X¹ is —C(O)—N(R), N(R)—C(O), (O—CH₂—CH₂)ₘ—, —O(C₆H₄)—, —(O—CH₂—CH₂—CH₂)ₚ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; each of m, n, and p is independently an integer from one to three; and Y is as described above.

In some embodiments, Ring B1 is

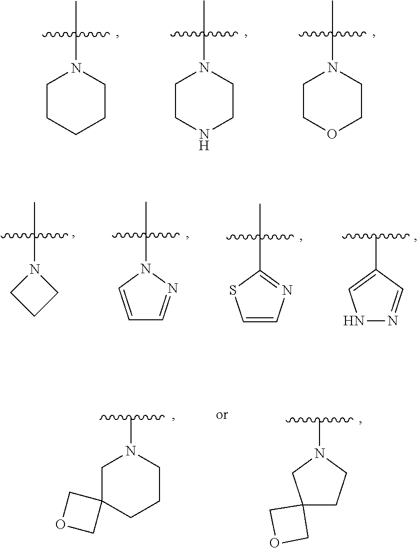

and Ring B1 is optionally substituted one to three groups selected from —CH₃, —CH₂OH, —CH₂CH₂OH, —C(O)OH, —CF₃, —F,

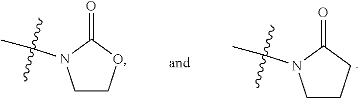 [SMILES: *N1CCOC1=O ; *N1CCCC1=O]

For example, Ring B1 is

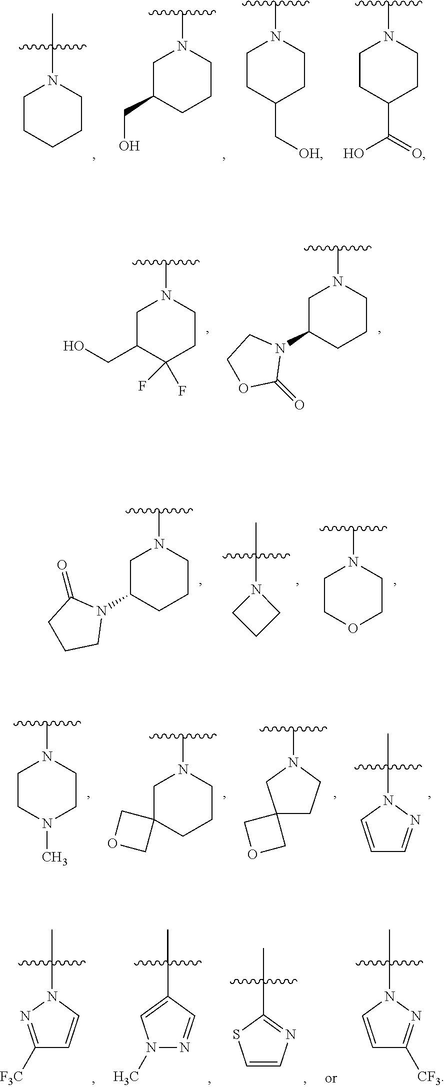

In other examples, Ring B1 is

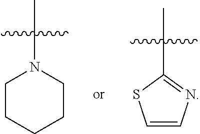 [SMILES: *N1CCCCC1 ; *c1nccs1]

In some embodiments, X¹ is,

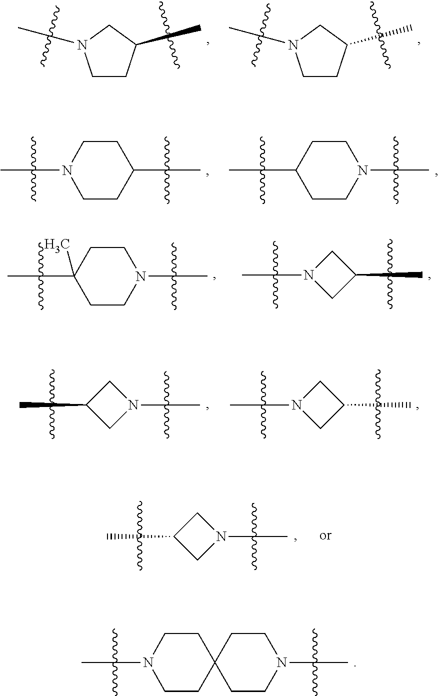

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond, —C₁₋₃ alkyl-, —C(O)—,

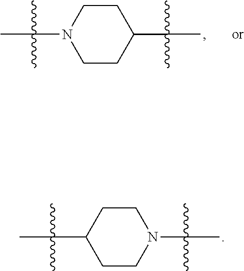 [SMILES: *C1CCN(*)CC1 ; *C1CCN(*)CC1]

In some embodiments, X³ is a bond, —C₁₋₄ alkyl-, —N(R)—, —(O—CH₂—CH₂)ₚ—, —(CH₂—CH₂—O)ₚ—, or a 4-6 membered heterocycloalkyl having one to two heteroatoms independently selected from N, O, or S, wherein the heterocycloalkyl is optionally substituted with —CH₃. For example, X³ is a bond,

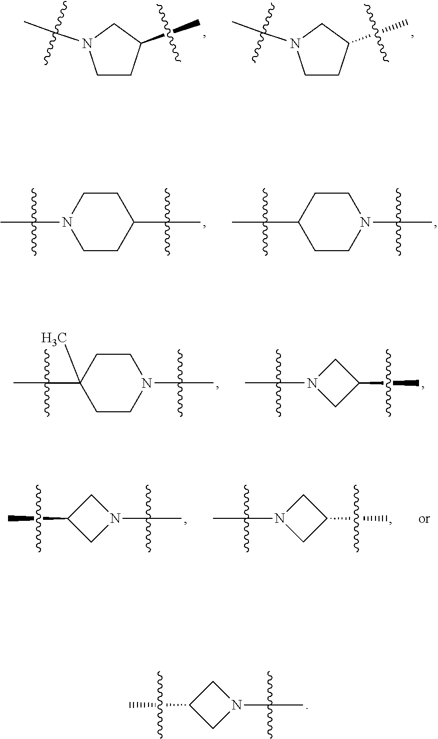

In some embodiments, L is

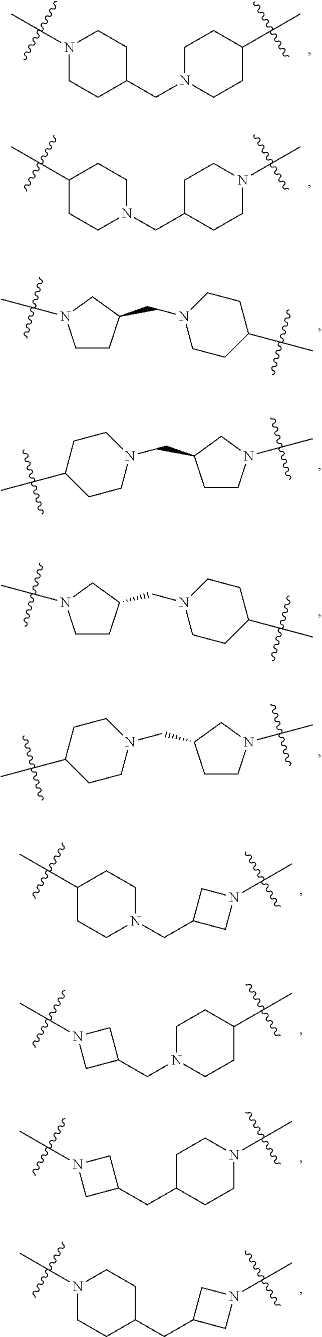

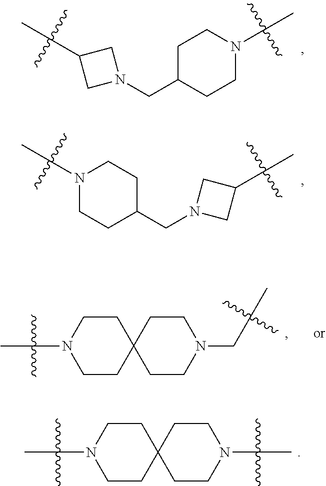 [SMILES: *C1CN(CC2CCN(*)CC2)C1 ; *C1CN(CC2CCN(*)CC2)C1 ; *CN1CCC2(CCN(*)CC2)CC1 ; *N1CCC2(CC1)CCN(*)CC2]

In some embodiments, W is N and D is a bond.

This disclosure also provides a compound of Formula (C)

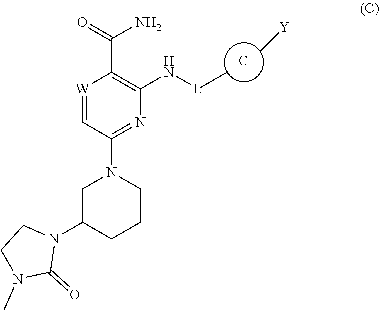

or a pharmaceutically acceptable salt thereof, wherein W is CH or N; Ring C is phenyl or a saturated, partially unsaturated, or fully unsaturated 5-6 membered monocyclic heterocycle having one to two heteroatoms independently selected from N, O, or S, wherein each of the phenyl and heterocycle of Ring C is optionally substituted; L is —X¹—X²—X³—; X¹ is —C(O)—N(R), N(R)—C(O), (O—CH₂—CH₂)ₘ, —O—(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the bicyclic heterocycloalkyl and the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; and each of m, n, and p is independently an integer from one to three.

In some embodiments, W is N.

In some embodiments Ring C is

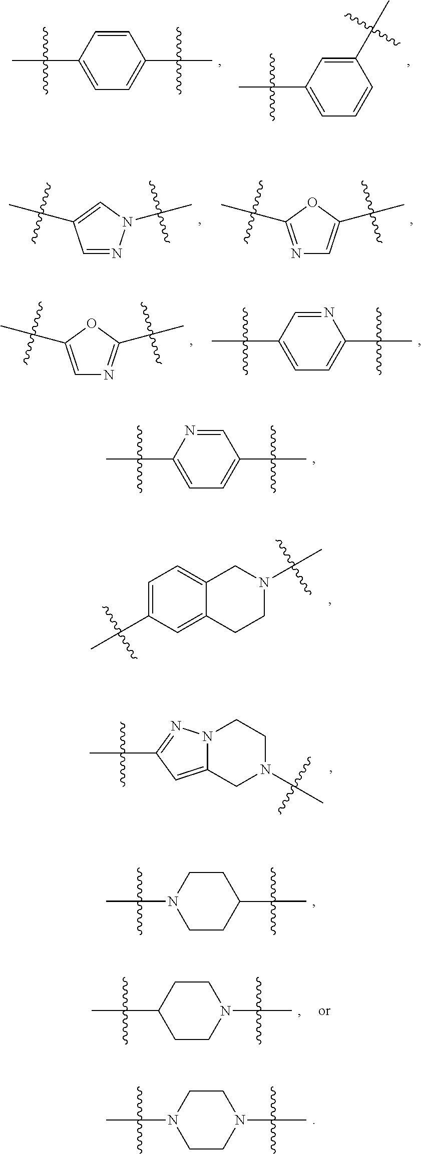

For example, Ring C is

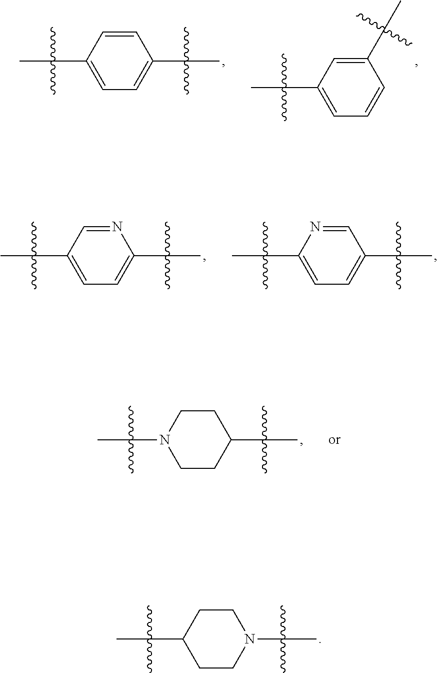 [SMILES: *c1ccc(*)cc1 ; *c1cccc(*)c1 ; *c1ccc(*)nc1 ; *c1ccc(*)nc1 ; *C1CCN(*)CC1 ; *C1CCN(*)CC1]

In other examples, Ring C is

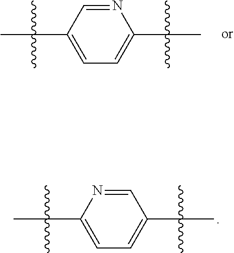 [SMILES: *c1ccc(*)nc1 ; *c1ccc(*)nc1]

In some embodiments, X¹ is a 4-6 membered monocyclic heterocycloalkyl having one to two heteroatoms independently selected from N, O, or S. For example, X¹ is

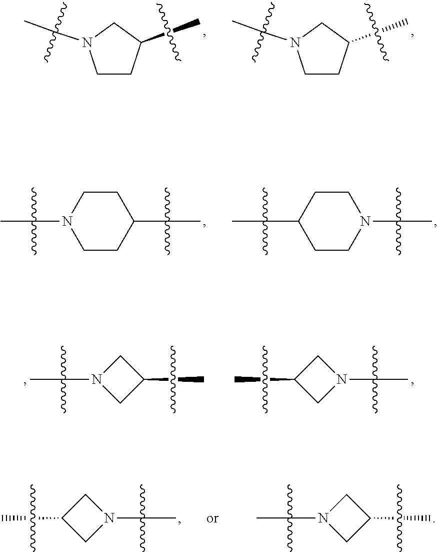

In some examples, X¹ is

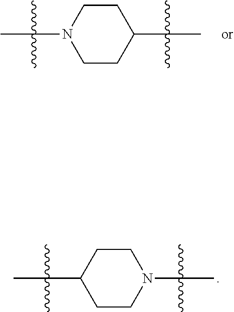 [SMILES: *C1CCN(*)CC1 ; *C1CCN(*)CC1]

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₃ alkyl- (e.g., —CH₂—).

In some embodiments, X³ is a 4-6 membered cycloalkyl, —N(R)—, or a 4-6 membered heterocycloalkyl having one to two heteroatoms independently selected from N, O, or S, wherein the heterocycloalkyl is optionally substituted with —CH₃. For example, X³ is

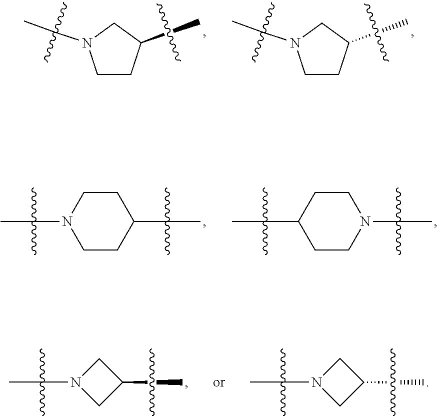

In other embodiments X³ is

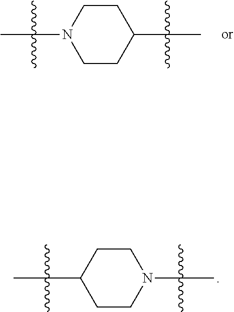 [SMILES: *C1CCN(*)CC1 ; *C1CCN(*)CC1]

In some embodiments, L is

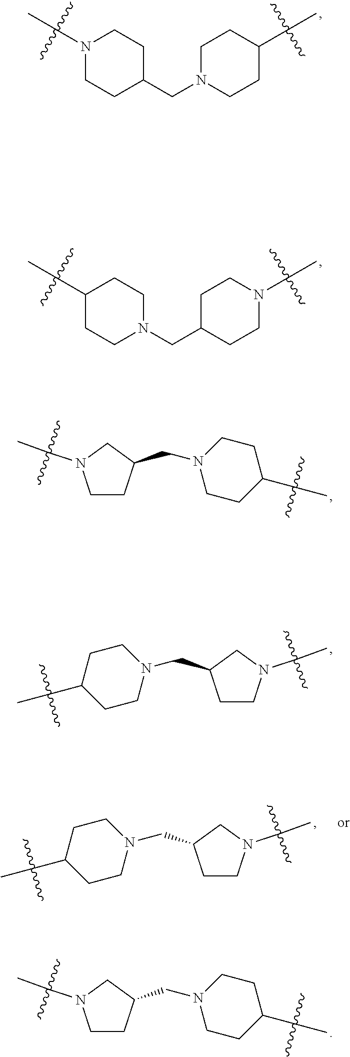 [SMILES: *C1CCN(CC2CCN(*)CC2)CC1 ; *C1CCN(CC2CCN(*)CC2)CC1 ; *C1CCN(C[C@H]2CCN(*)C2)CC1 ; *C1CCN(C[C@@H]2CCN(*)C2)CC1 ; *C1CCN(C[C@H]2CCN(*)C2)CC1 ; *C1CCN(C[C@@H]2CCN(*)C2)CC1]

For example, L is

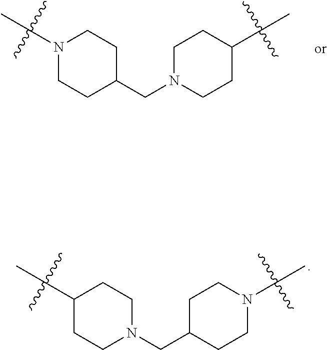 [SMILES: *C1CCN(CC2CCN(*)CC2)CC1 ; *C1CCN(CC2CCN(*)CC2)CC1]

This disclosure also provides a compound of Formula (D)

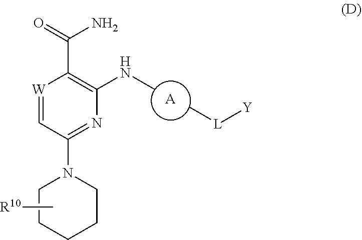

or a pharmaceutically acceptable salt thereof, wherein W is CH or N; Ring A is

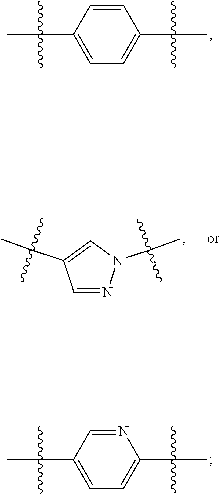 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having one to two heteroatoms independently selected from N, O, or S, wherein the monocyclic heterocycloalkyl of X¹ is optionally substituted with —CH₃; X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃;Y is as described herein; and R¹⁰ is halo, —H, —C₁₋₅ alkyl, 3-6 membered cycloalkyl, 5-6 membered heterocycloalkyl, —CN, —OH, —CF₃, —CH₂OH, —CH₂CH₂OH, —C(O)OH,

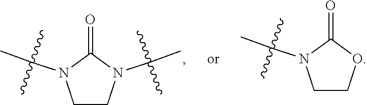 [SMILES: *N1CCN(*)C1=O ; *N1CCOC1=O]

In some embodiments, Ring A is

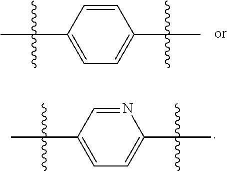

In some embodiments, X¹ is a 4-6 membered monocyclic heterocycloalkyl having one to two heteroatoms independently selected from N, O, or S, wherein the monocyclic heterocycloalkyl of X¹ is optionally subsituted with —CH₃. For example, X¹ is

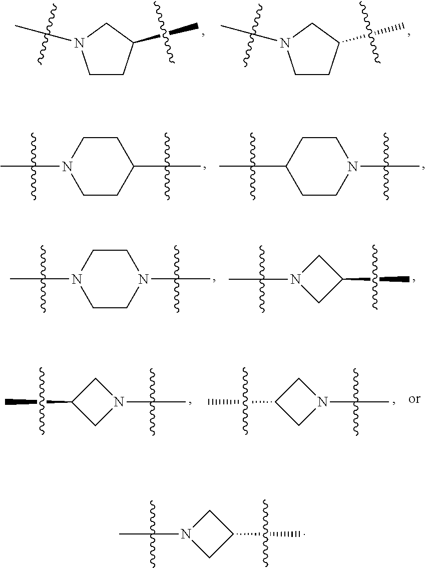

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O, or S. For example, X³ is

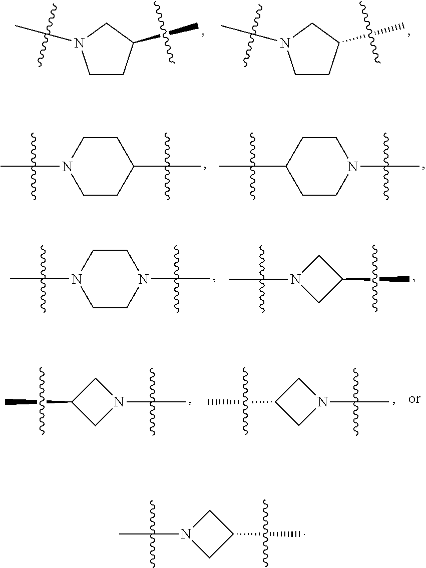

In some embodiments, L is

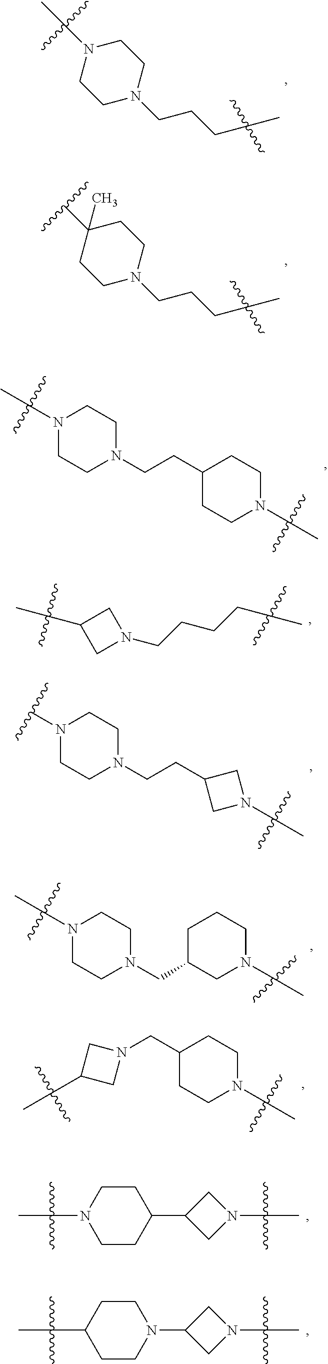

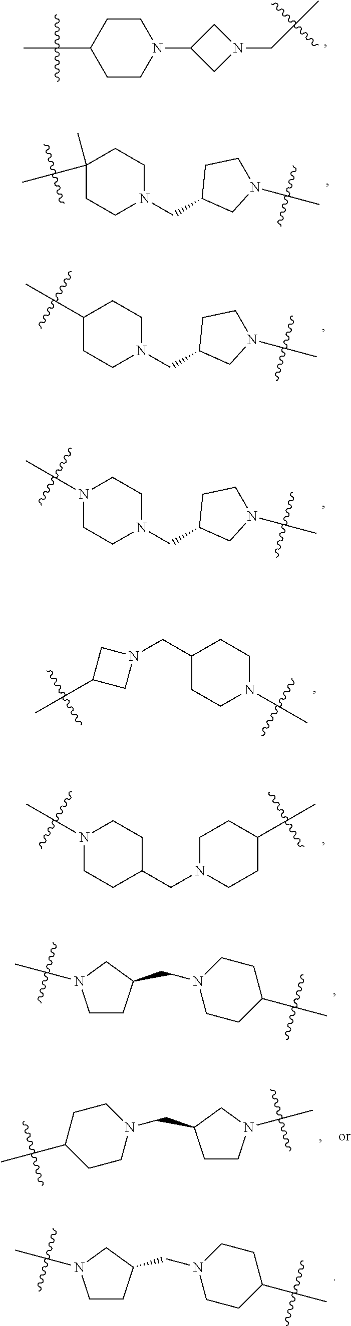

In some embodiments, R¹⁰ is halo, —H, —C₁₋₅ alkyl (e.g., —C₁₋₃ alkyl), 3-6 membered cycloalkyl, 5-6 membered heterocycloalkyl, —CN, —OH, —CF₃, —CH₂OH, —C(O)OH, or —CH₂CH₂OH. For instance, R¹⁰ is halo, —H, C₁₋₃ alkyl, CF₃, —CH₂OH, —C(O)OH, or —CH₂CH₂OH. In other instances, R¹⁰ is

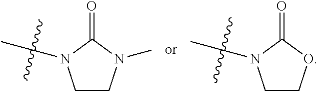

In some embodiments, R¹⁰ is

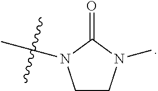 [SMILES: *N1CCN(C)C1=O]

In some embodiments, R¹⁰ is

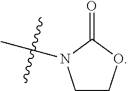 [SMILES: *N1CCOC1=O]

In some embodiments, the compound of Formula (D) is a compound of (D-1)

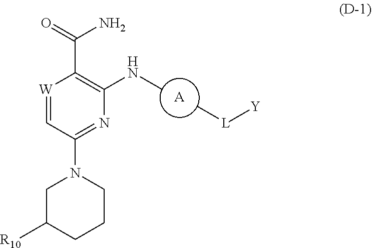

or a pharmaceutically acceptable salt thereof, wherein W is CH or N; Ring A is

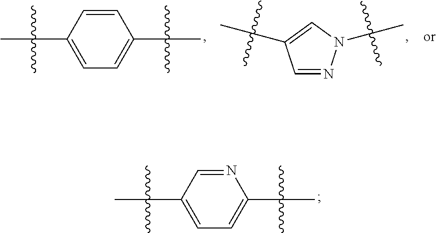 [SMILES: *c1ccc(*)cc1 ; *c1cnn(*)c1 ; *c1ccc(*)nc1]

L is —X¹—X²—X³— X¹ is —C₁₋₅ alkyl- or 4-6 membered monocyclic heterocycloalkyl having one to two heteroatoms independently selected from N, O, or S, wherein the monocyclic heterocycloalkyl of X¹ is optionally substituted with —CH₃; X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃; Y is as described herein; and R¹⁰ is

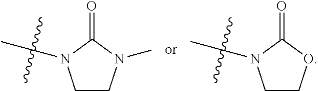

In some embodiments, Ring A is

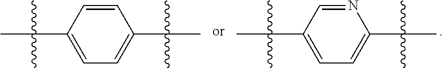 [SMILES: *c1ccc(*)cc1 ; *c1ccc(*)nc1]

In some embodiments, X¹ is a 4-6 membered monocyclic heterocycloalkyl having one to two heteroatoms independently selected from N, O, or S, wherein the monocyclic heterocycloalkyl of X¹ is optionally substituted with —CH₃. For example, X¹ is

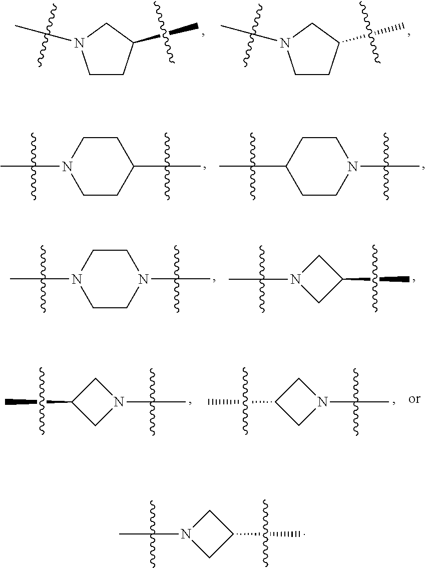

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O, or S. For example, X³ is

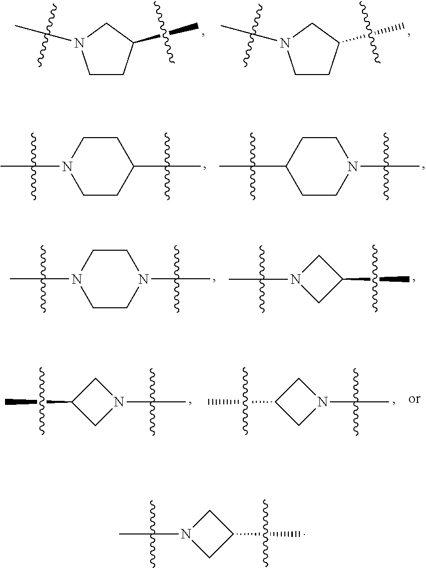

In some embodiments, L is

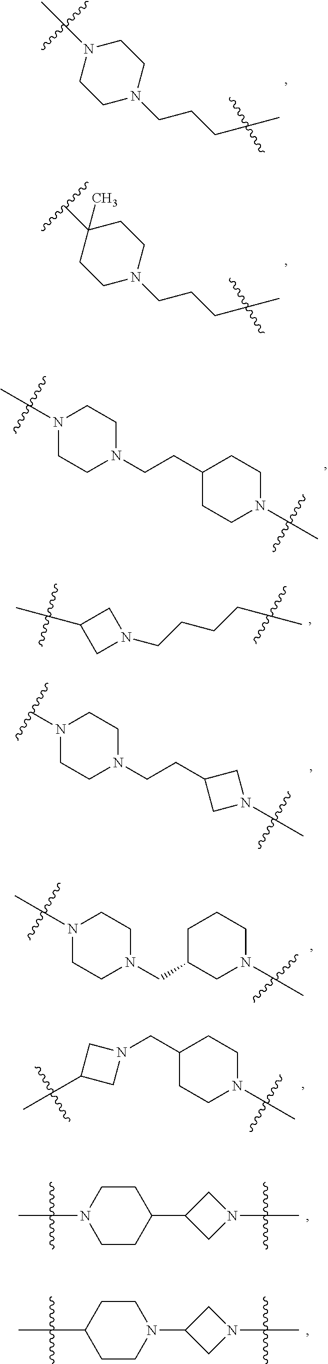

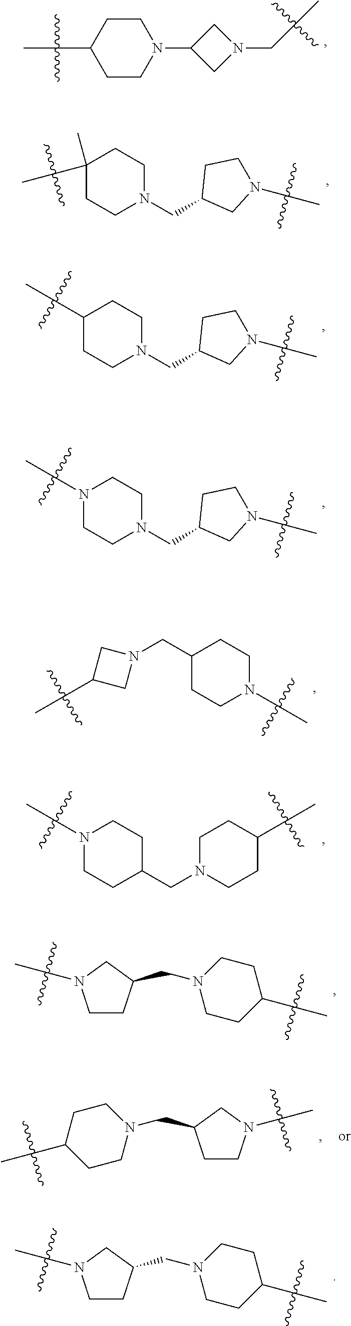

In some embodiments, R¹⁰ is

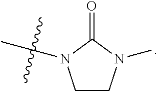 [SMILES: *N1CCN(C)C1=O]

In some embodiments, R¹⁰ is

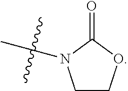 [SMILES: *N1CCOC1=O]

In some embodiments, the compound of Formula (D) or the compound of Formula (D-1) is a compound of Formula (D-2)

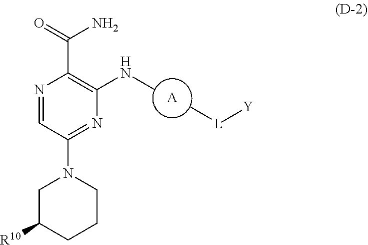

or a pharmaceutically acceptable salt thereof, wherein the terms Ring A, L, Y, and R¹⁰ are as defined in the compound of Formula (A), the compound of Formula (D), and the compound of Formula (D-1).

In some embodiments, Ring A is

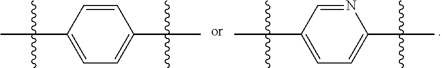 [SMILES: *c1ccc(*)cc1 ; *c1ccc(*)nc1]

In some embodiments, X¹ is a 4-6 membered monocyclic heterocycloalkyl having one to two heteroatoms independently selected from N, O, or S, wherein the monocyclic heterocycloalkyl of X¹ is optionally substituted with —CH₃. For example, X¹ is

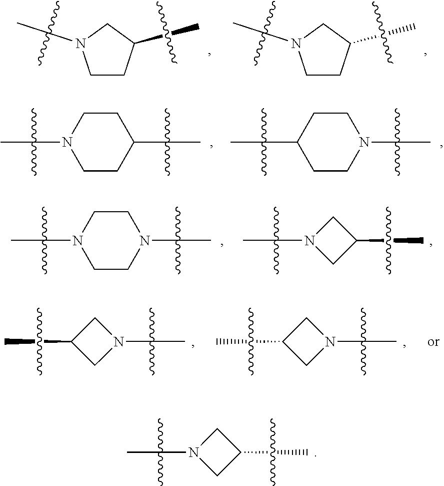

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O, or S. For example, X³ is

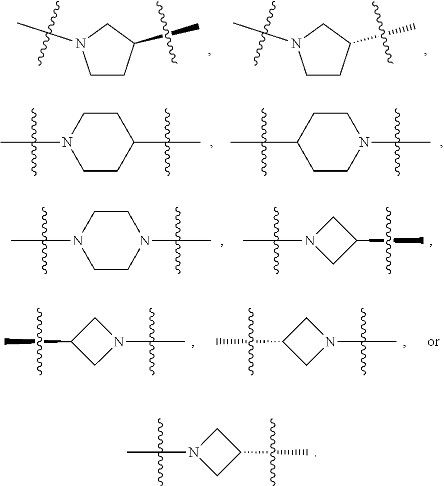

In some embodiments, L is

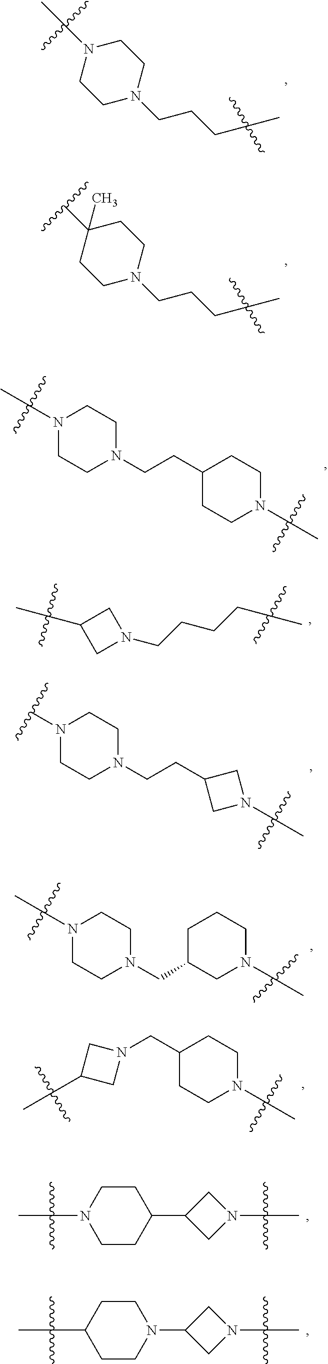

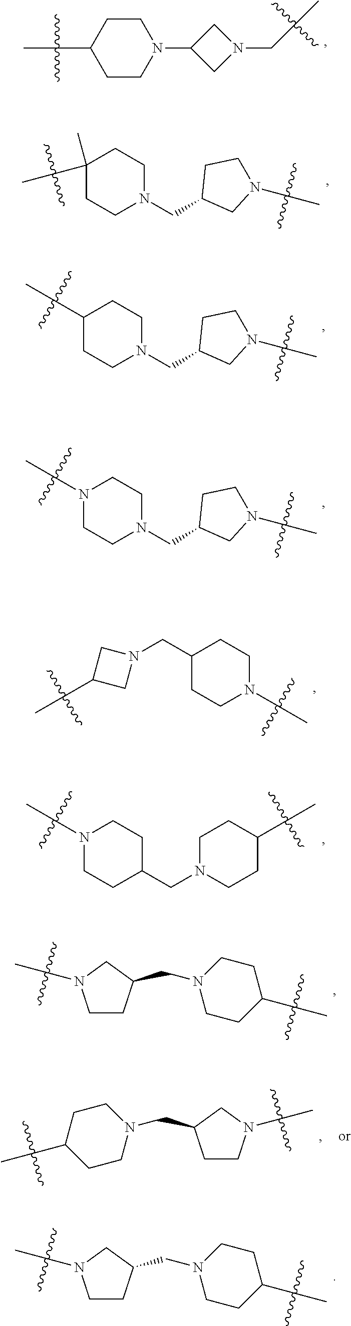

In some embodiments, R¹⁰ is

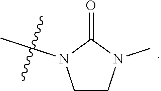 [SMILES: *N1CCN(C)C1=O]

In some embodiments, R¹⁰ is

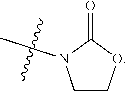 [SMILES: *N1CCOC1=O]

This disclosure also provides a compound of Formula (E)

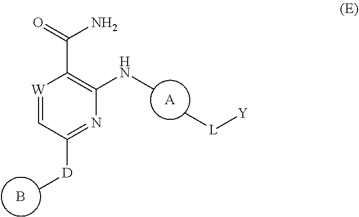

or a pharmaceutically acceptable salt thereof, wherein D is a bond or —NH—; W is N or CH; Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S; Ring B is an optionally substituted 5-6 membered saturated, partially unsaturated, or fully unsaturated monocyclic heterocycle, or an optionally substituted 8-10 membered (e.g., 8-9 membered or 9-10 membered) spiro bicyclic heterocycle, wherein Ring B has one to three heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S; X⁵ is a bond, —N(R)—, or —C(O)—N(R)—; each R is independently —H or —C₁₋₃ alkyl; each of m, n, and p is independently an integer from one to three; and Y is as described herein, wherein at least one of X¹, X², X³, X⁴, and X⁵ has a nitrogen atom, and Y is directly bonded to L at a nitrogen atom of X¹, X², X³, X⁴, or X⁵.

In some embodiments, Ring B is

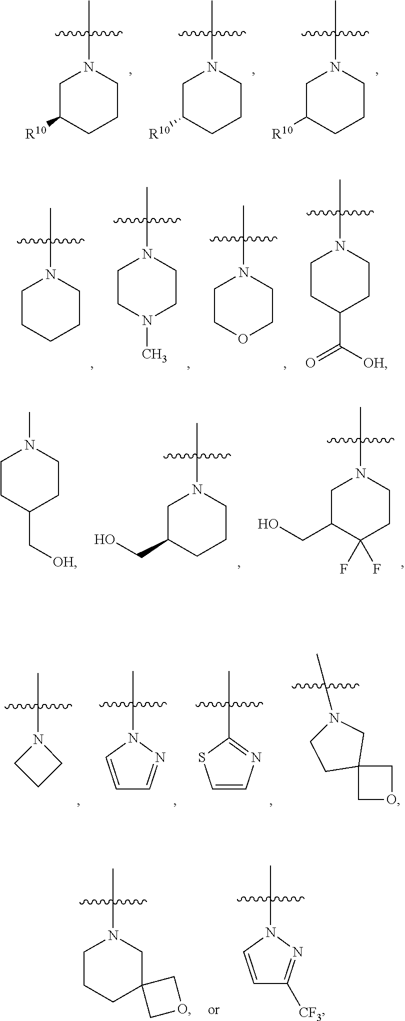

wherein R¹⁰ is

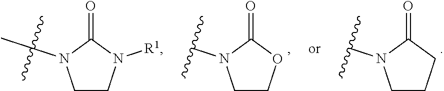

and wherein R¹ is a C₁₋₄ alkyl group. For example, Ring B is

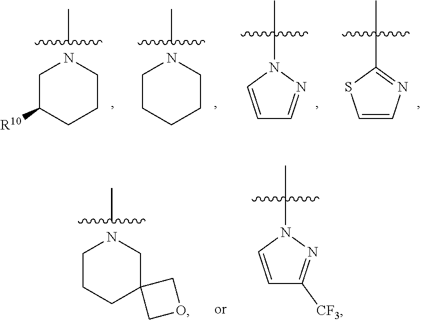

wherein R¹⁰ is

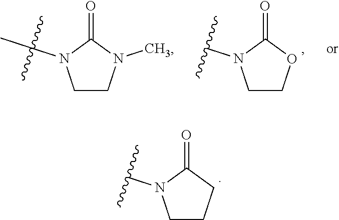 [SMILES: *N1CCN(C)C1=O ; *N1CCOC1=O ; *N1CCCC1=O]

In other examples, Ring B is

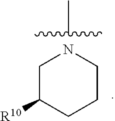

In some embodiments, R¹⁰ is

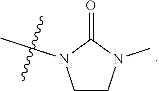 [SMILES: *N1CCN(C)C1=O]

In some embodiments, Ring A is

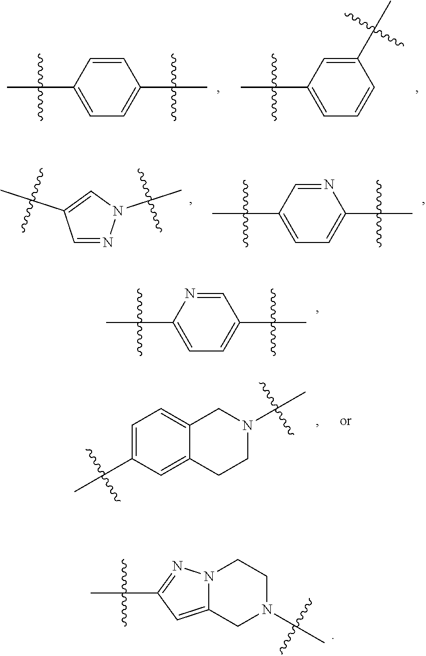

In some embodiments, X⁵ is —N(R)—.

In some embodiments, X⁵ is —C(O)—N(R)—.

In some embodiments, X⁵ is a bond.

In some embodiments, L is

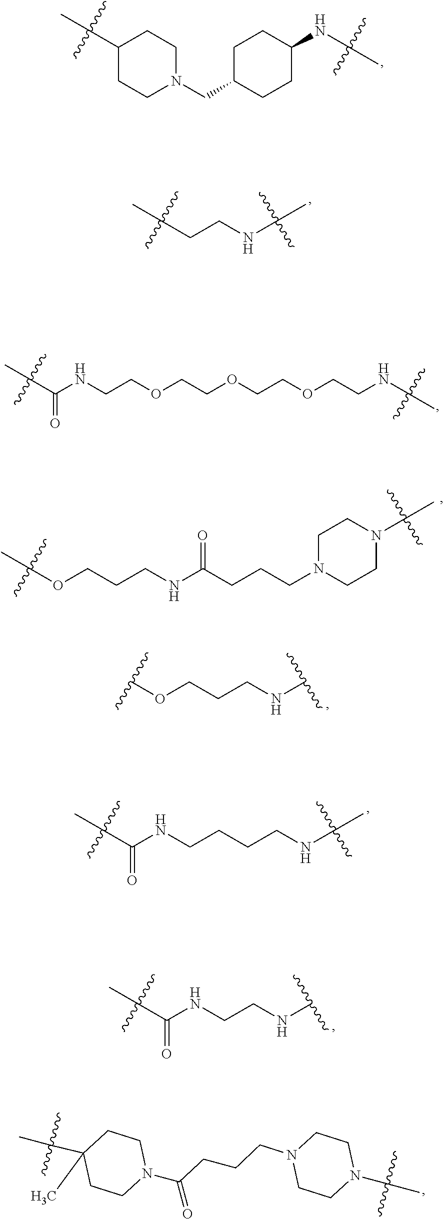

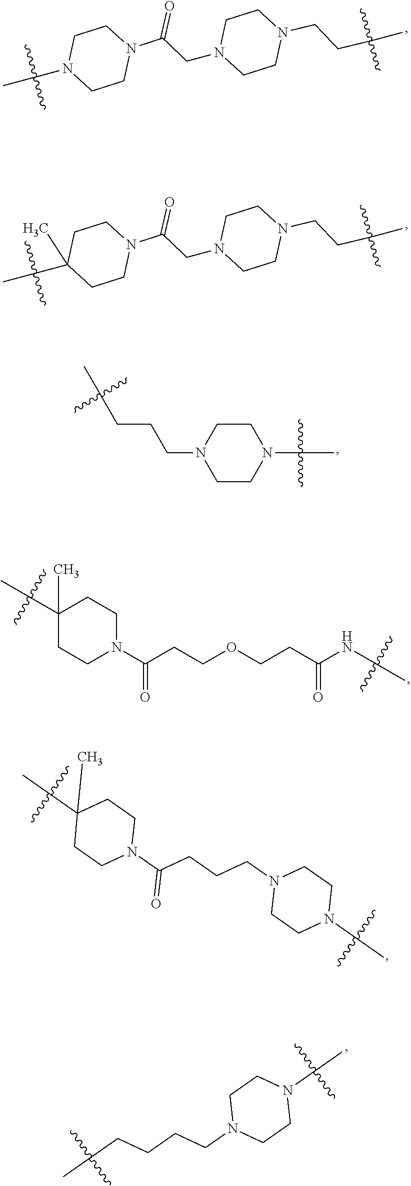

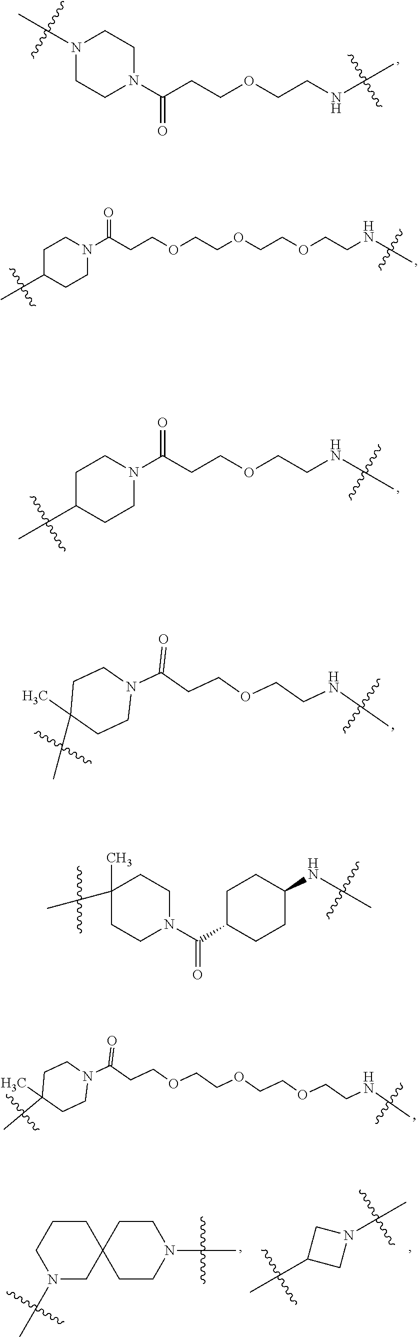

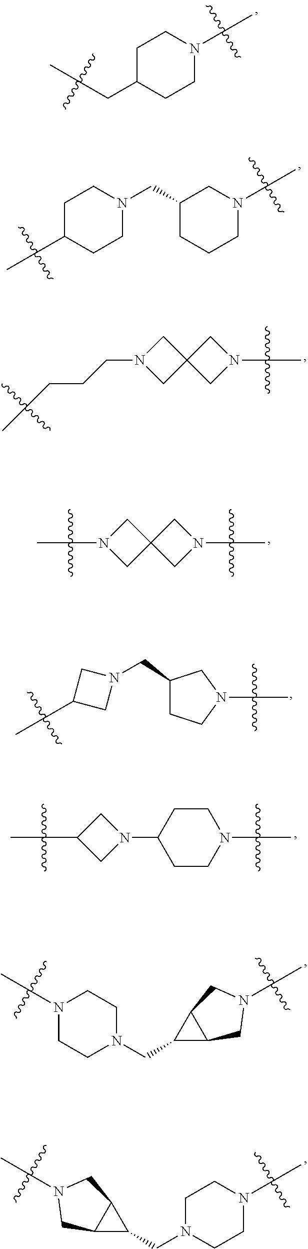

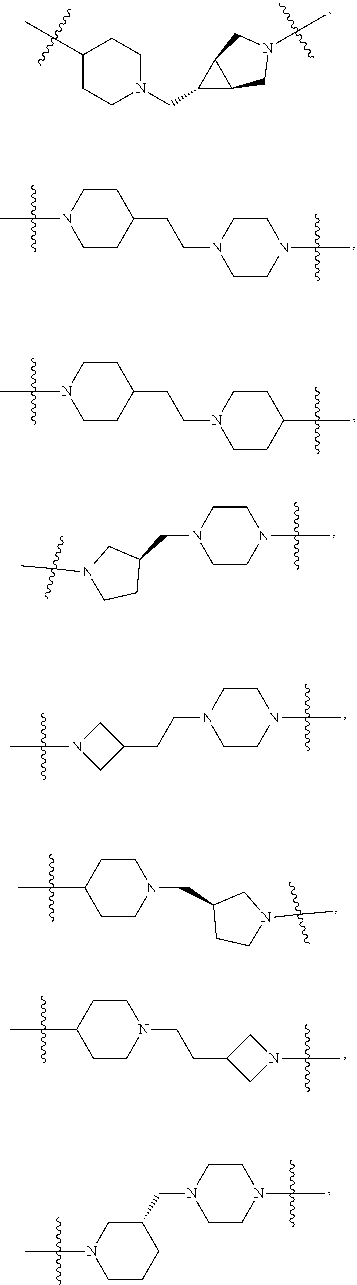

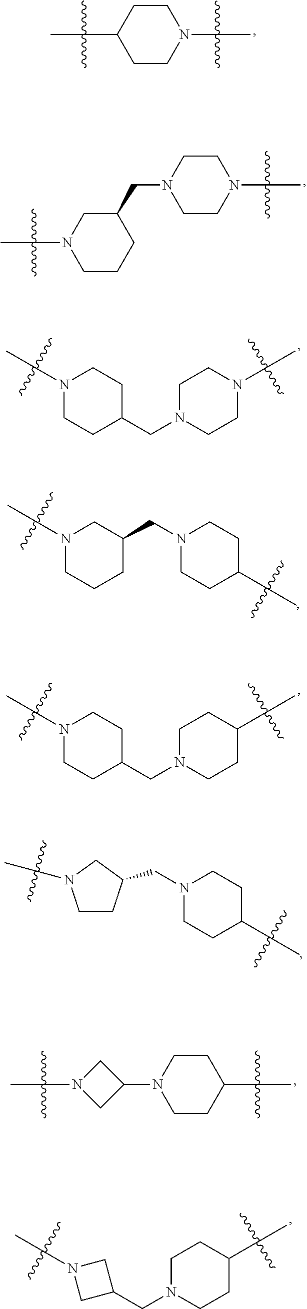

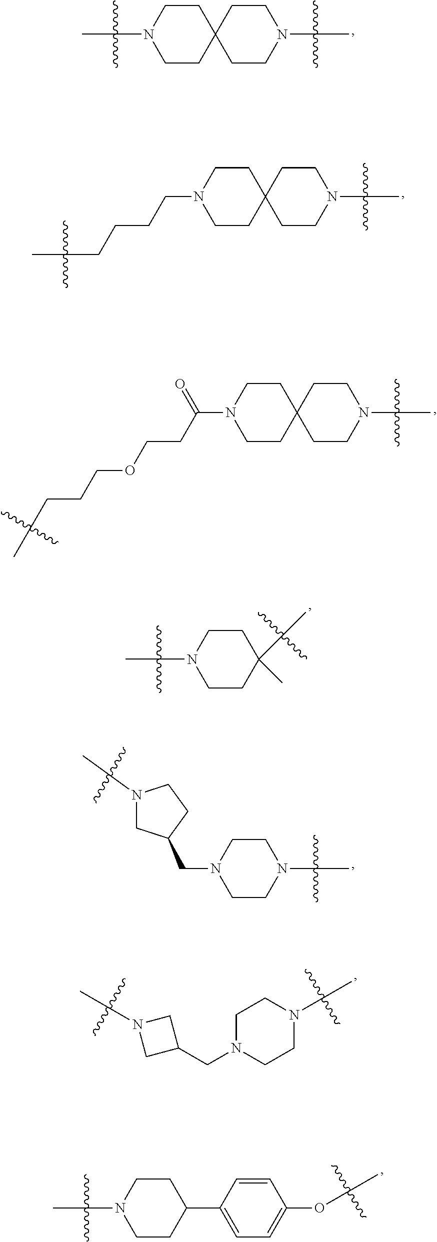

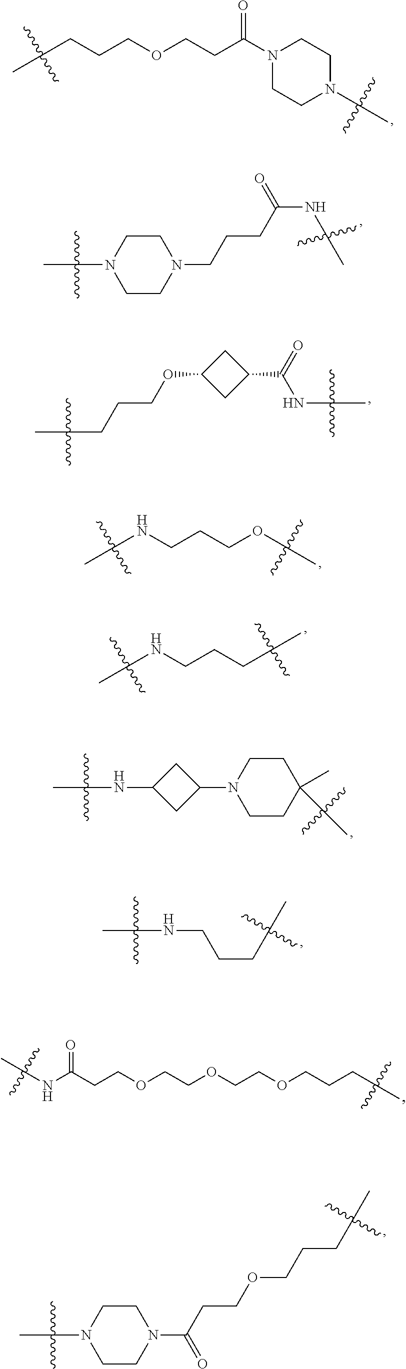

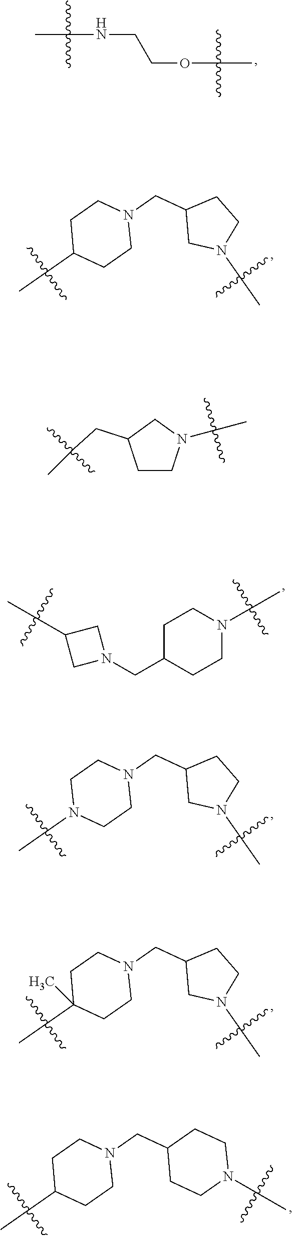

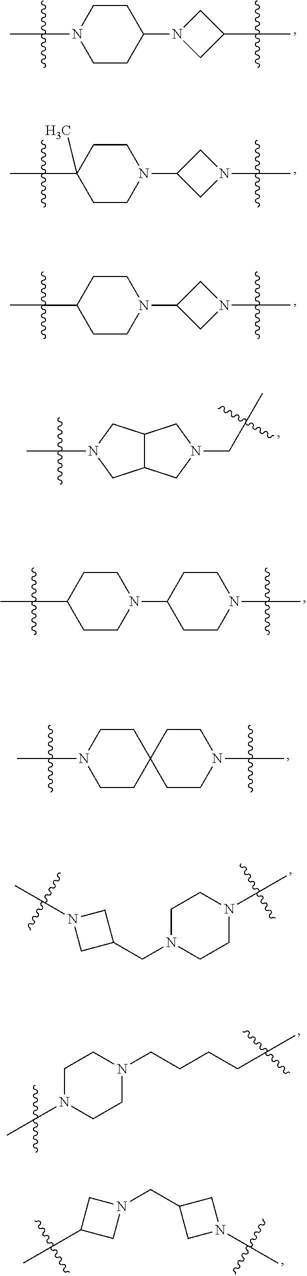

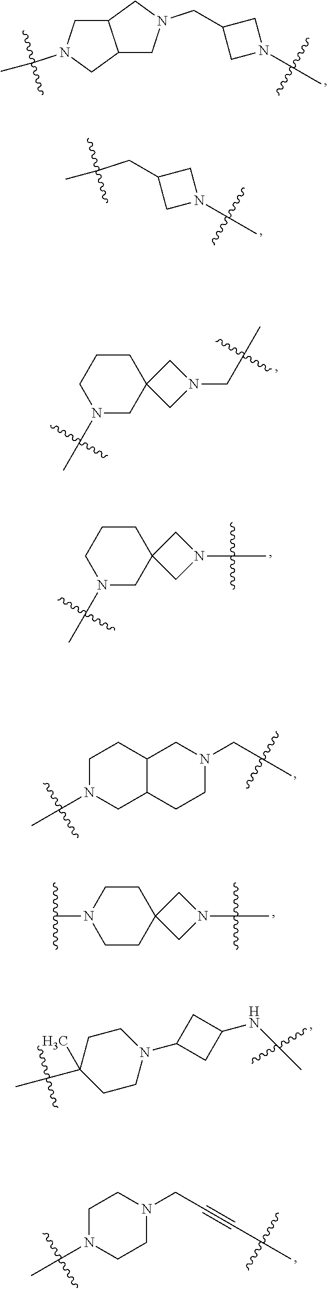

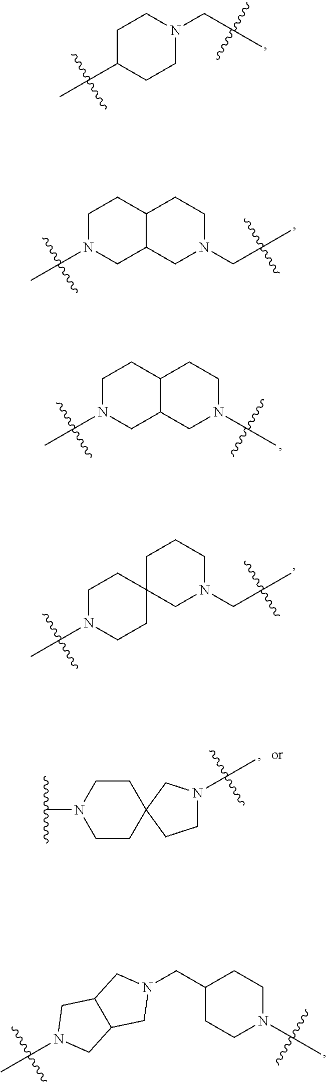 [SMILES: *CN1CCC(*)CC1 ; *CN1CCC2CCN(*)CC2C1 ; *N1CCC2CCN(*)CC2C1 ; *CN1CCCC2(CCN(*)CC2)C1 ; *N1CCC2(CC1)CCN(*)C2 ; *N1CCC(CN2CC3CN(*)CC3C2)CC1]

This disclosure also provides a compound of Formula (F)

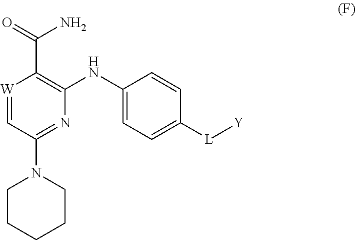

or a pharmaceutically acceptable salt thereof, wherein W is CH or N; L is —X¹—X²—X³—; X¹ is —C(O)—N(R), N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —C₁₋₅ alkyl-, —(O—CH₂—CH₂)ₙ (CH₂—CH₂—O)ₙ, N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; each of m, n, and p is independently an integer from one to three; and Y is as described herein.

In some embodiments, W is N.

In some embodiments, X¹ is a 4-6 membered monocyclic heterocycloalkyl having one to two heteroatoms independently selected from N, O, or S, wherein each of the monocyclic heterocycloalkyl of X¹ is optionally substituted with —CH₃. For example, X¹ is

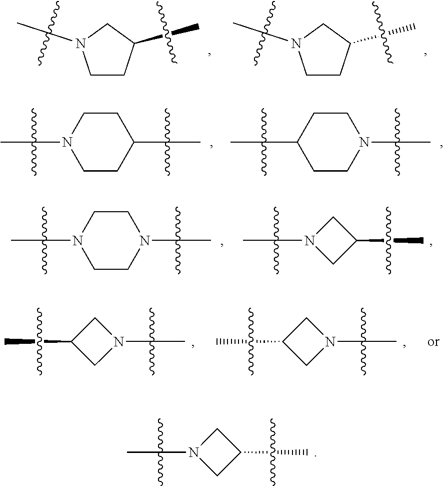

In some instances, X¹ is

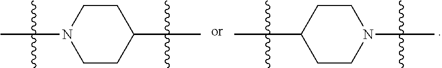 [SMILES: *C1CCN(*)CC1 ; *C1CCN(*)CC1]

In some embodiments, X² is a bond or —C₁₋₅ alkyl-.

In some embodiments, X³ is a 4-6 membered monocyclic heterocycloalkyl having one N to two heteroatoms independently selected from N, O, or S. For example, X³ is

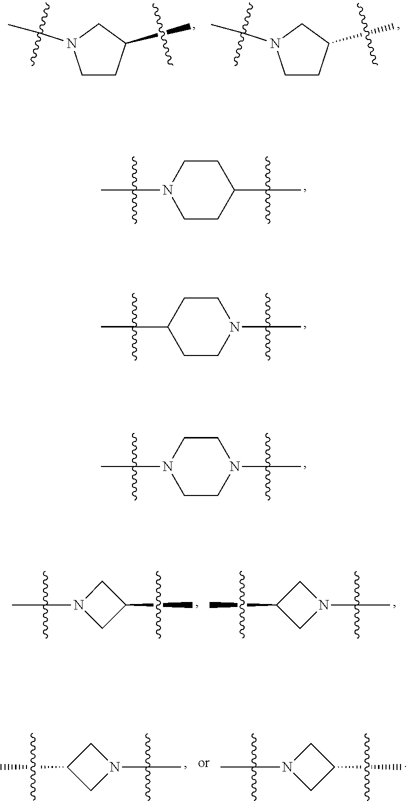

In some instances, X³ is

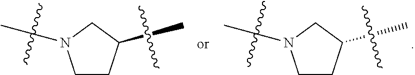

In some embodiments, L is

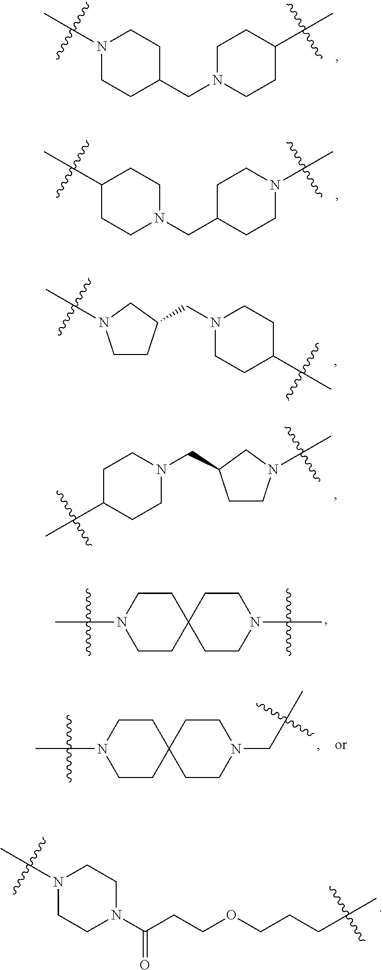

In some embodiments, L is

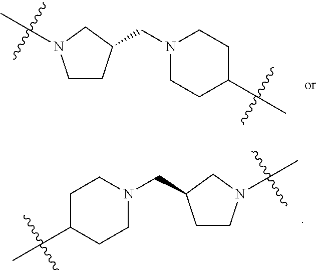 [SMILES: *C1CCN(C[C@@H]2CCN(*)C2)CC1 ; *C1CCN(C[C@@H]2CCN(*)C2)CC1]

In some embodiments, W is N; and L is

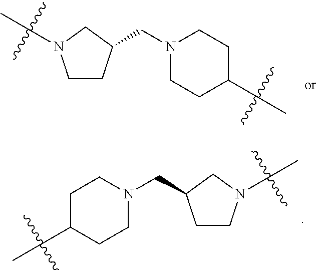 [SMILES: *C1CCN(C[C@@H]2CCN(*)C2)CC1 ; *C1CCN(C[C@@H]2CCN(*)C2)CC1]

This disclosure also provides a compound of Formula (G)

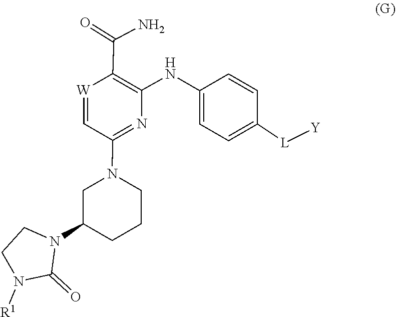

or a pharmaceutically acceptable salt thereof wherein R¹, L, and Y are as defined for compounds of Formula (A).

In some embodiments, R¹ is methyl.

In some embodiments, W is N.

This disclosure also provides a compound of Formula (M)

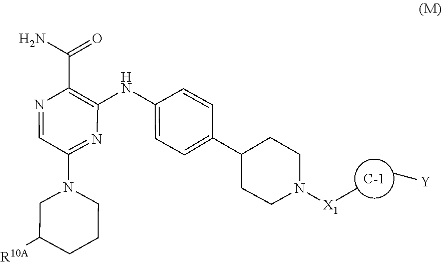

or a pharmaceutically acceptable salt thereof, wherein R^{10A} is —H,

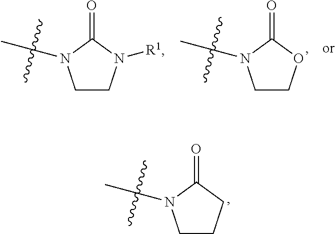

wherein R¹ is C₁₋₄ alkyl; X¹ is —C₁₋₅ alkyl-; Ring C-1 is a 5-6 membered heterocycloalkyl having one nitrogen atom; and Y is as described herein.

In some embodiments, R^{10A} is —H or

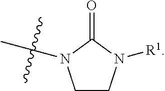

In some embodiments, R^{10A} is

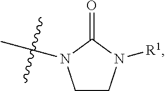

and R¹ is methyl, ethyl, propyl, iso-propyl, butyl, sec-butyl, or iso-butyl. For example, R¹ is methyl.

In some embodiments, X¹ is methylene (—CH₂—), ethylene (—CH₂CH₂—), or propylene (—CH₂CH₂CH₂—). For instance, X¹ is methylene (—CH₂—).

In some embodiments, Ring C-1 is

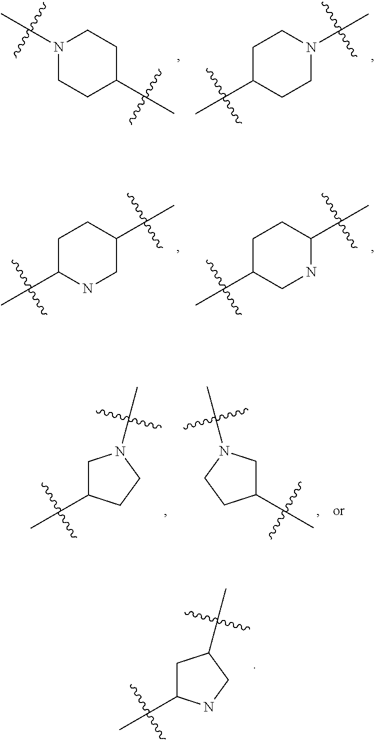

For instance, Ring C-1 is

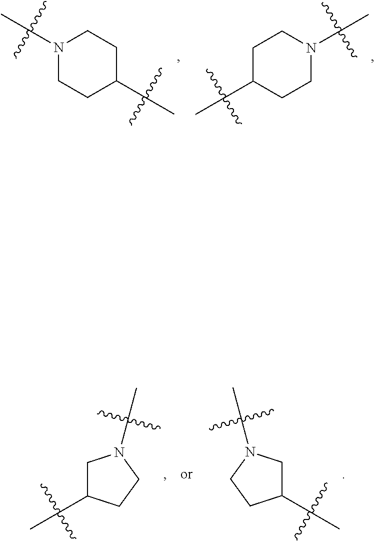 [SMILES: *C1CCN(*)CC1 ; *C1CCN(*)CC1 ; *C1CCN(*)C1 ; *C1CCN(*)C1]

This disclosure provides a compound of Formula (X)

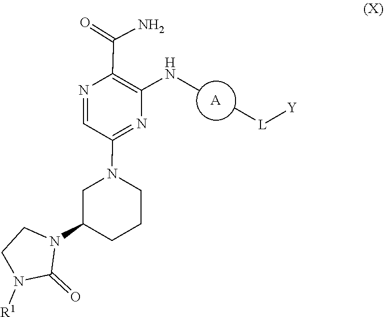

or a pharmaceutically acceptable salt thereof, wherein R¹ is C₁₋₃ alkyl; Ring A is phenyl, 5-6 membered partially or fully unsaturated monocyclic heterocycle, 9-10 membered bicyclic aryl, or 9-10 membered bicyclic heteroaryl, wherein the heterocycle and the bicyclic heteroaryl of Ring A each independently have one to three heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the bicyclic heterocycloalkyl of X¹ is optionally substituted with —CH₃, or 4-6 membered monocyclic heterocycloalkyl having one to two heteroatoms independently selected from N, O, or S, wherein the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated carbocycle having zero to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or C₁₋₄ alkyl; each Z is —C(R^{A})₂— or —C(O)—; each R^{A} is independently —H or C₁₋₄ alkyl; and q is zero, one, or two.

In some instances, the compound of Formula (X) is a compound of Formula (I)

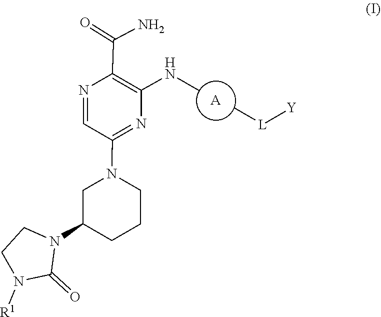

or a pharmaceutically acceptable salt thereof, wherein R¹ is C₁₋₃ alkyl; Ring A is phenyl, 9-10 membered bicyclic aryl, or 9-10 membered bicyclic heteroaryl having one to three heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three (e.g., one, two, or three); Y is as described herein, wherein each R² is independently halo or —C₁₋₄ alkyl; each Z is —C(R^{A})₂- or —C(O)—; each R^{A} is independently —H or —C₁₋₄ alkyl; and q is zero, one, or two.

In some embodiments, q is zero. In other embodiments, q is one and R² is —F.

In some embodiments, Z is —CH₂— or —C(O)—.

In some embodiments, R¹ is —C₁₋₃ alkyl. For example, R¹ is methyl, ethyl, propyl, or iso-propyl. In other embodiments, R¹ is methyl.

In some embodiments, each R is independently —H or —CH₃. For instance, each R is —H.

In some embodiments, X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered heterocycloalkyl having one to two heteroatoms independently selected from N, O, or S, wherein the heterocycloalkyl is optionally substituted with —CH₃. In some embodiments, X¹ is —C(O)—N(R)—. For example, X¹ is —C(O)—N(H)—, —C(O)—N(CH₃)—, or —C(O)—N(CH₂CH₃)—. In other embodiments, X¹ is a 5-6 membered heterocycloalkyl having one to two heteroatoms independently selected from N, O, or S, wherein the heterocycloalkyl is optionally substituted with —CH₃. For example, X¹ is,

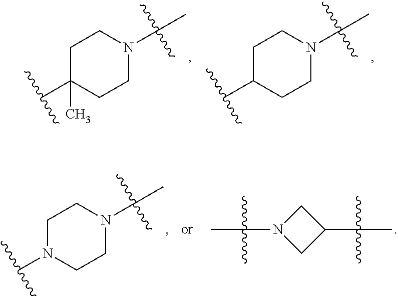

In other examples, X¹ is a 7-10 membered spiro bicyclic heterocycloalkyl ring having one to three heteroatoms independently selected from N, O, or S (e.g., N). For example, X¹ is

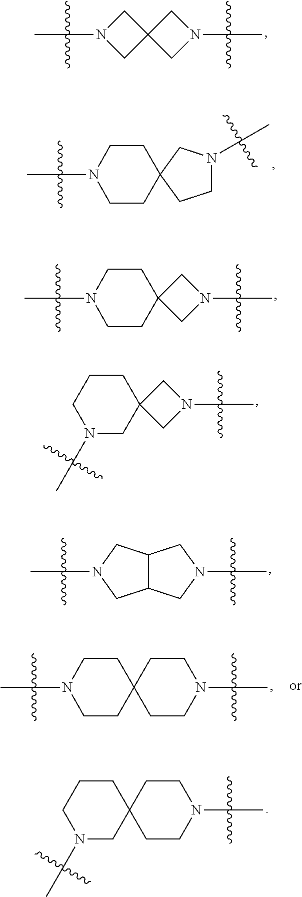

In other embodiments, X¹ is —(O—CH₂—CH₂)ₘ— or —(O—CH₂—CH₂—CH₂)ₘ—, wherein m is one, two, three. For example, X¹ is —(O—CH₂—CH₂)ₘ— or —(O—CH₂—CH₂—CH₂)ₘ—, and m is one. In another example, X¹ is —(O—CH₂—CH₂)ₘ— or —(O—CH₂—CH₂—CH₂)ₘ—, and m is two. In some embodiments, X¹ is —C₁₋₅ alkyl-. For example, X¹ is methylene (—CH₂—), ethylene (—CH₂CH₂—), propylene (—CH₂CH₂CH₂—), butylene (—CH₂CH₂CH₂CH₂—), or the like. In some embodiments, X¹ is —CH₂—, —C(O)—,

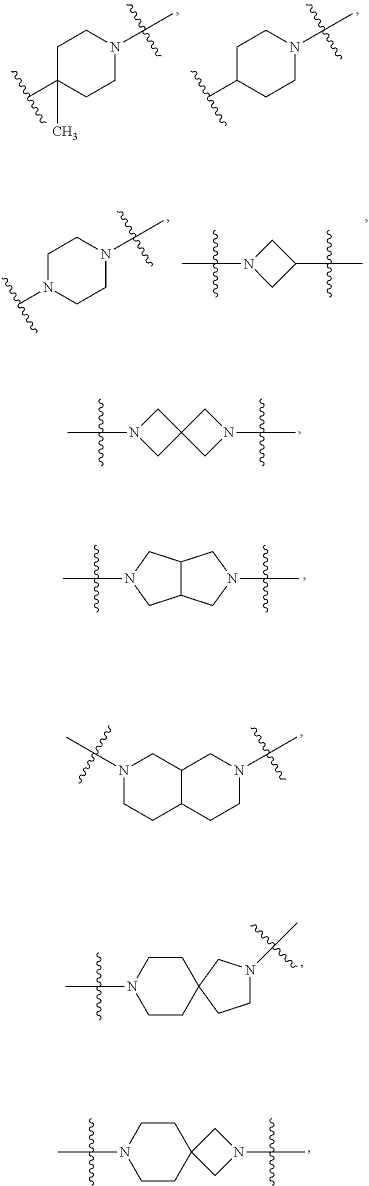

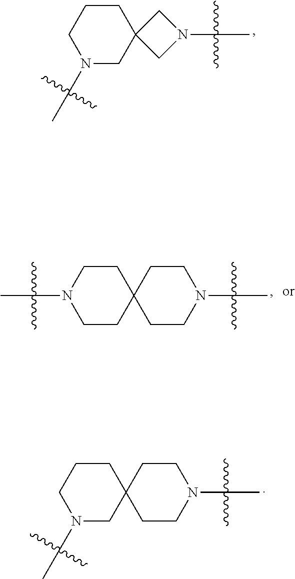 [SMILES: *N1CCCC2(C1)CN(*)C2 ; *N1CCC2(CC1)CCN(*)CC2 ; *N1CCC2(CCCN(*)C2)CC1]

In some embodiments, X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S. In some embodiments, X² is a bond. In some embodiments, X² is —(O—CH₂—CH₂)ₙ—, —(CH₂—CH₂—O)ₙ—, or —C₁₋₅ alkyl-, wherein n is one, two, or three. For example, X¹ is —C(O)—N(R)—, and X² is —(O—CH₂—CH₂)ₙ—, —(CH₂—CH₂—O)ₙ—, or —C₁₋₅ alkyl-. In some examples, X² is —(O—CH₂—CH₂)ₙ— or —(CH₂—CH₂—O)ₙ—, where n is one or two. In other examples, X² is —C₁₋₅ alkyl-. For instance, X² is methylene (—CH₂—), ethylene (—CH₂CH₂—), propylene (—CH₂CH₂CH₂—), butylene (—CH₂CH₂CH₂CH₂—), or the like. In other examples, X² is a bond, —CH₂—, —CH₂CH₂—, or —CH₂CH₂CH₂—. In some examples, X² is 4-6 membered cycloalkyl. For instance, X² is

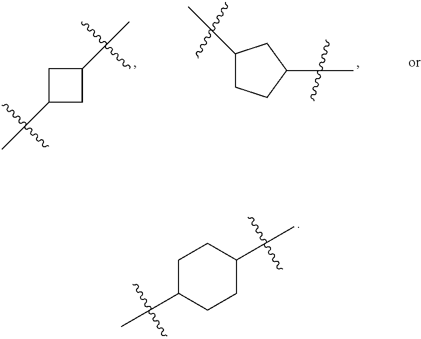 [SMILES: *C1CC(*)C1 ; *C1CCC(*)C1 ; *C1CCC(*)CC1]

In other examples X² is 4-6 membered heterocycloalkyl having one to two heteroatoms independently selected from O, or S. For instance, X is

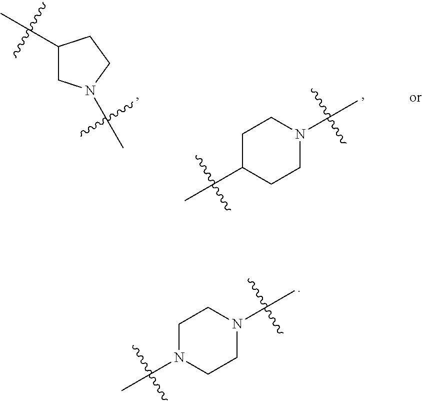 [SMILES: *C1CCN(*)C1 ; *C1CCN(*)CC1 ; *N1CCN(*)CC1]

In some embodiments, X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃. In some embodiments, X³ is a bond. In some embodiments, X³ is methyl, ethyl, propyl, iso-propyl, butyl, or the like. In some embodiments, X³ is cyclopentyl or cyclohexyl. In some embodiments, X³—N(H)—. And, in other embodiments, X³ is —(O—CH₂—CH₂)ₚ— or —(CH₂—CH₂—O)ₚ—, wherein p is one or two.

In some embodiments, X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S. In some embodiments, X⁴ is a bond,

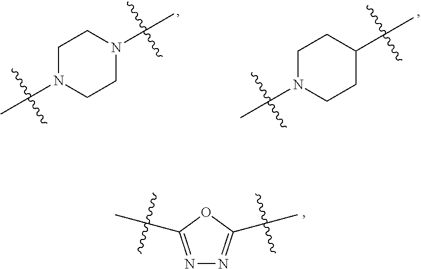 [SMILES: *N1CCN(*)CC1 ; *C1CCN(*)CC1 ; *c1nnc(*)o1]

—C₁₋₄ alkyl-, —CH₂—CH₂—N(R)—, or —N(R)—. For example, X⁴ is —CH₂—CH₂—N(H)—, or —N(H)—. In other examples, X⁴ is methyl, ethyl, propyl, iso-propyl, butyl, sec-butyl, or the like.

In some embodiments, X⁵ is a bond, —C₁₋₄ alkyl-, —N(R)—, or —C(O)—N(R)—. In some embodiments, X⁵ is a bond. In some embodiments, X⁵ is methyl, ethyl, propyl, iso-propyl, butyl, or the like. In some embodiments, X⁵ is —N(H)— or —C(O)—N(H)—.

In some embodiments, L is selected from H

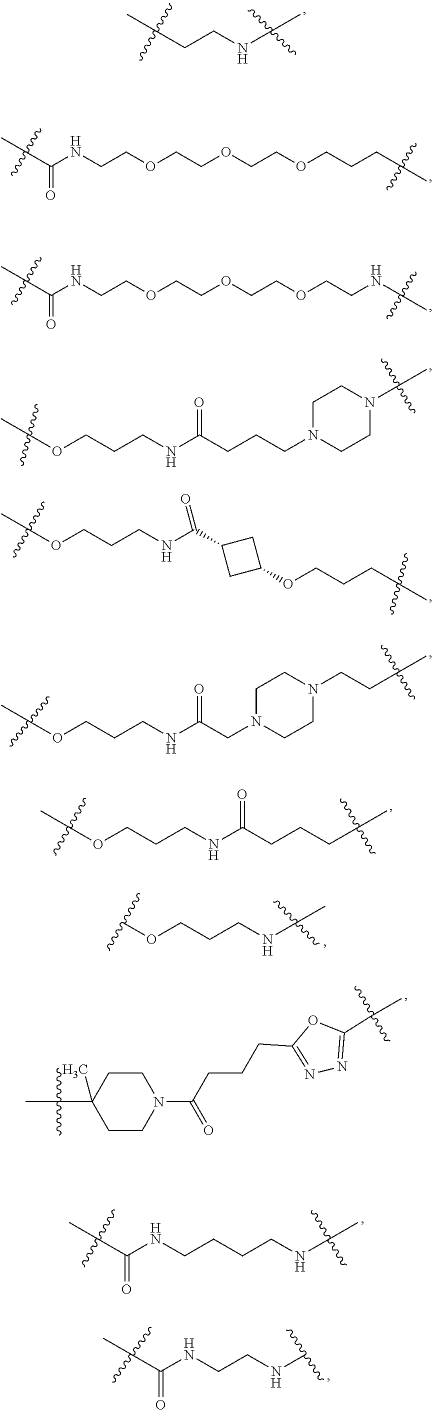

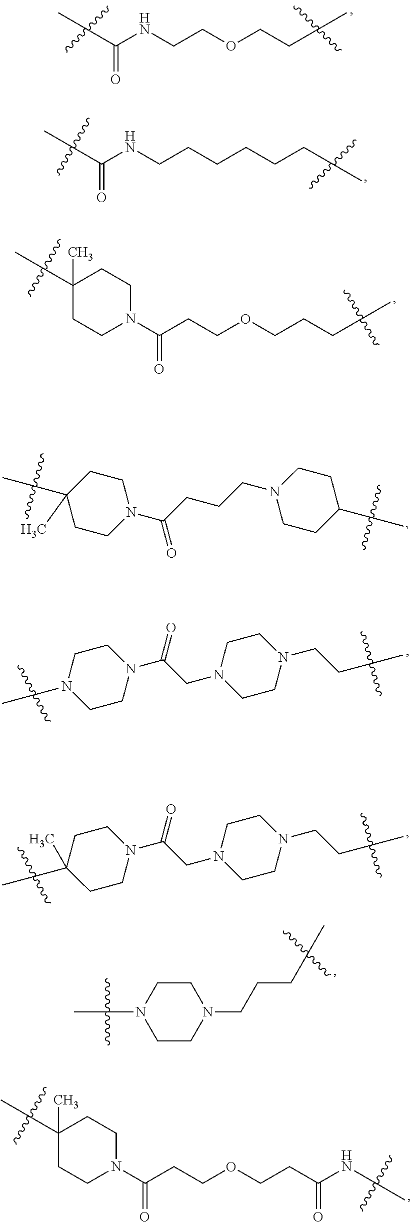

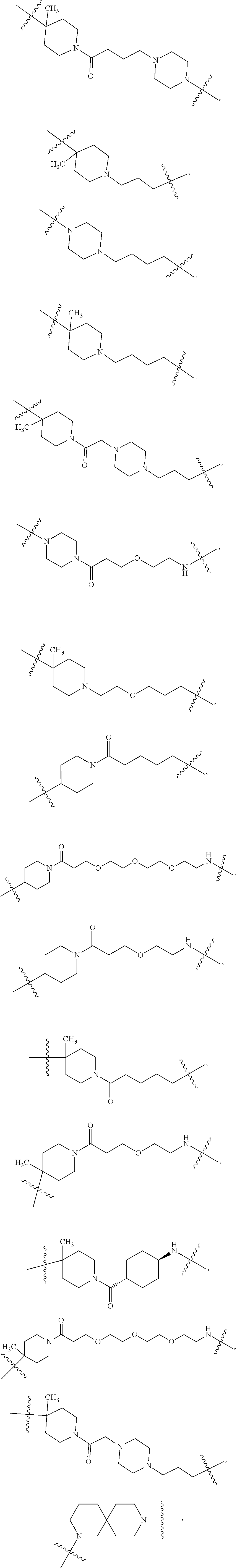

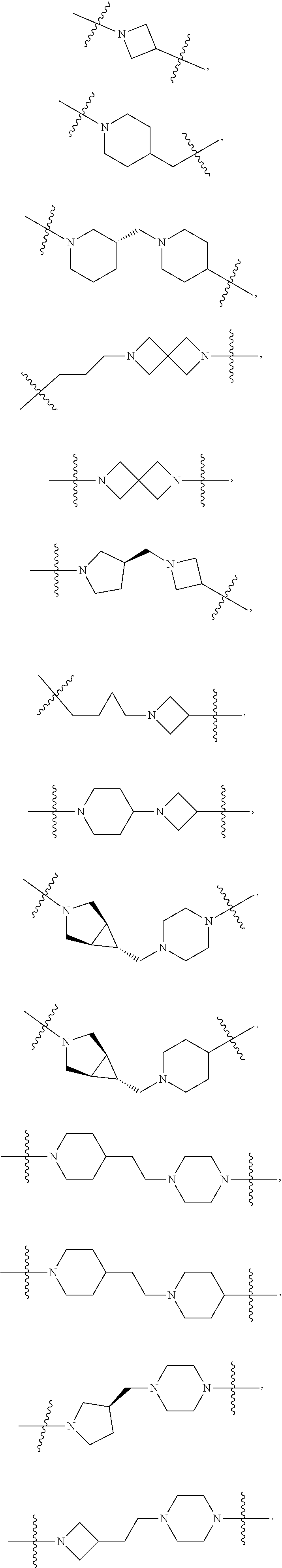

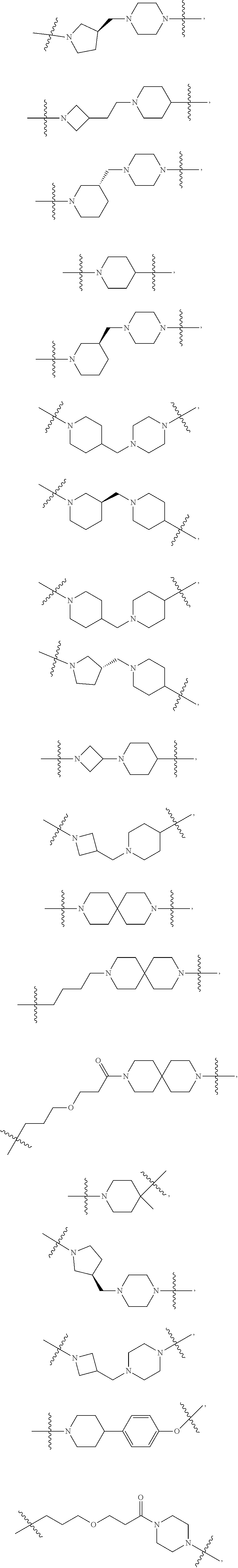

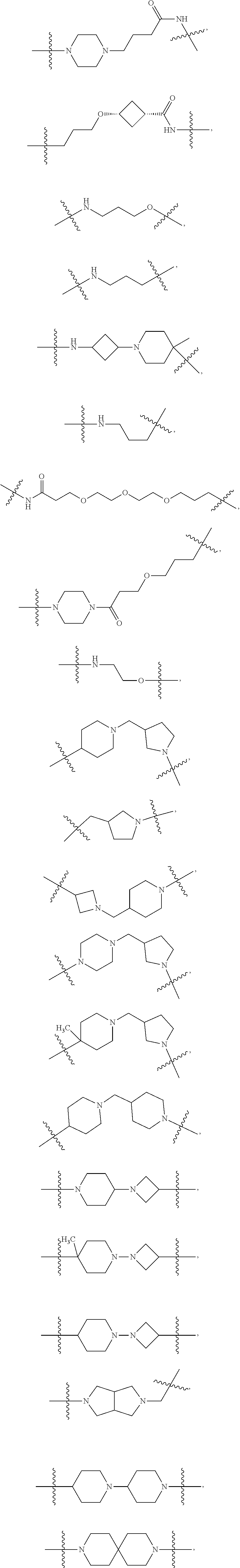

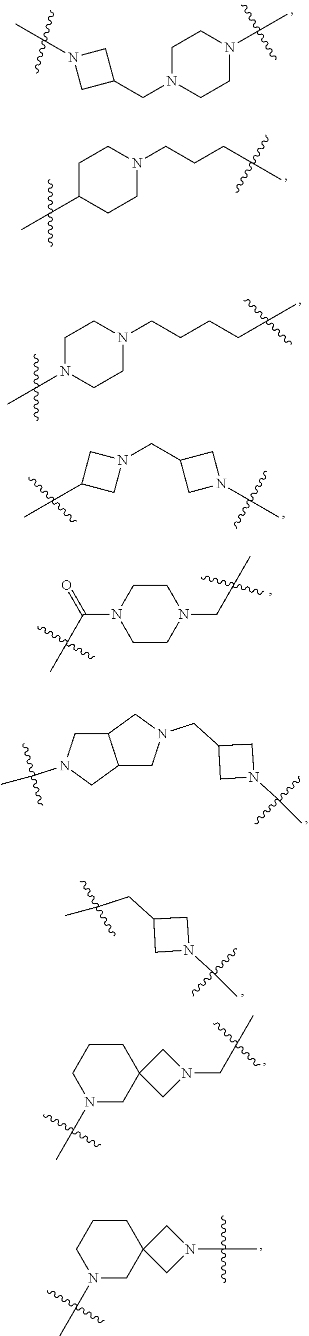

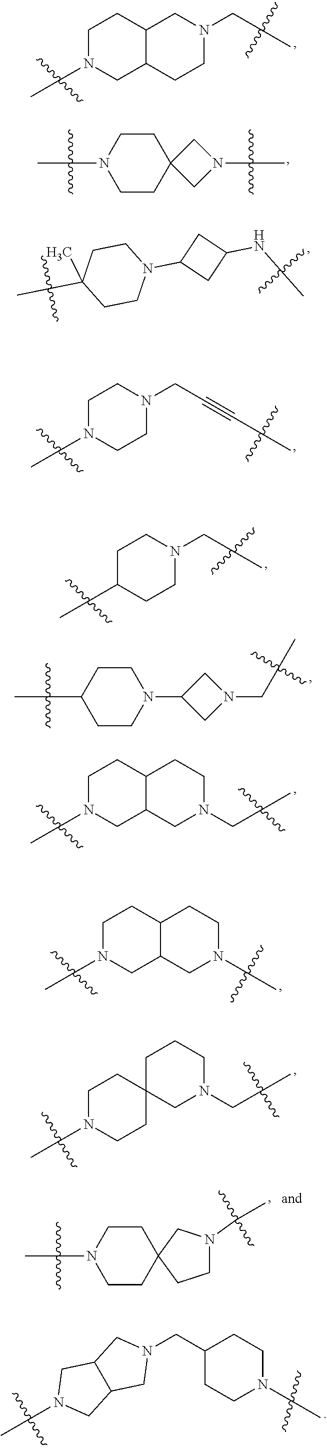

This disclosure also provides a compound of Formula (I-A):

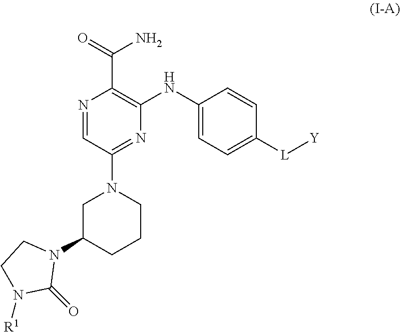

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O) (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or —C₁₋₄ alkyl; each Z is —C(R^{A})₂— or —C(O)—; each R^{A} is independently —H or —C₁₋₄ alkyl; and q is zero, one, or two.

In other embodiments, each of the variables in Formula (I-A) is as defined herein for the compound of Formula (X) or (I).

This disclosure also provides a compound of Formula (I-B)

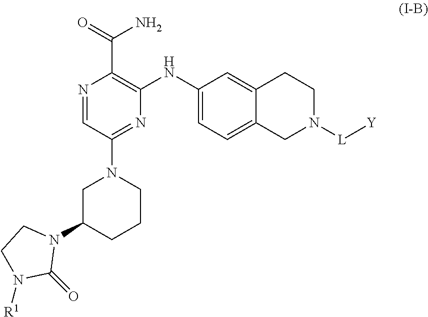

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl ring having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or C₁₋₄ alkyl; each Z is —C(R^{A})₂— or —C(O)—; each R^{A} is independently —H or C₁₋₄ alkyl; and q is zero, one, or two.

In other embodiments, each of the variables in Formula (I-B) is as defined herein for the compound of Formula (X) or (I).

This disclosure also provides a compound of Formula (III)

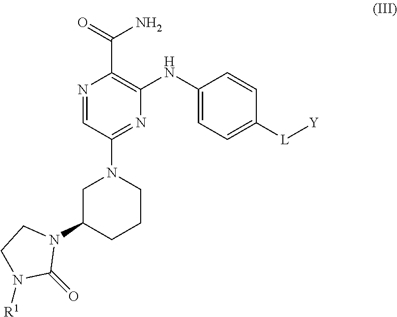

or a pharmaceutically acceptable salt thereof, wherein R¹ is C₁₋₃ alkyl; L is —X¹—X²—X³—; X¹ is 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond or —C₁₋₅ alkyl-; X³ is a bond, —C₁₋₄ alkyl-, 4-6 membered heterocycloalkyl having one to two heteroatoms independently selected from N, O, or S, wherein the heterocycloalkyl is optionally substituted with —CH₃; Y is as described herein, wherein each R² is independently halo or —C₁-4 alkyl; each Z is or —C(O)—; each R^{A} is independently —H; and q is zero, one, or two.

This disclosure also provides a compound of Formula (IV)

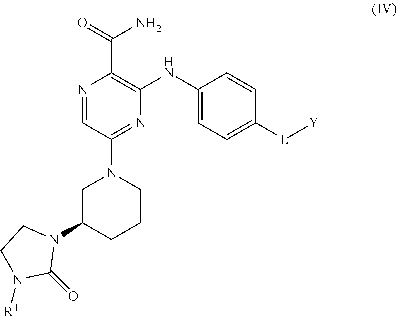

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O), (O—CH₂—CH₂)ₘ—, —O(C₆H₄)—, —(O—CH₂—CH₂—CH₂)ₘ—, —C₁₋₅ alkyl-, 7-12 membered Spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or —C₁₋₄ alkyl; each Z is —C(R^{A})₂— or —C(O)—; each R^{A} is independently —H or —C₁₋₄ alkyl; and q is zero, one, or two.

In certain embodiments of any of Formulas A-X or I-IV, Y is

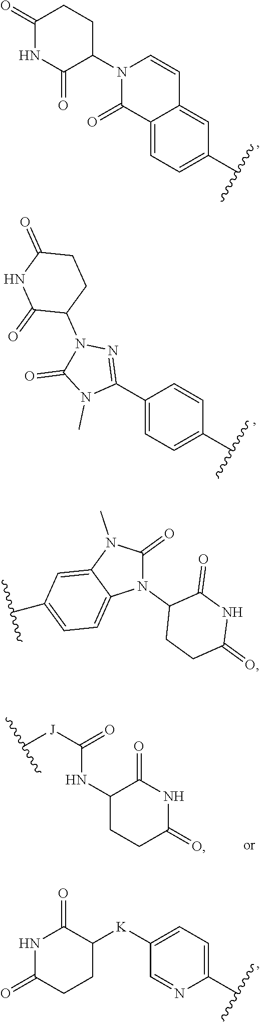

wherein each J is independently aryl or heteroaryl; and each K is independently absent, —CH₂—, —NH—, —NMe—, or —O—.

In certain embodiments of any of Formulas A-X or I-IV, Y is

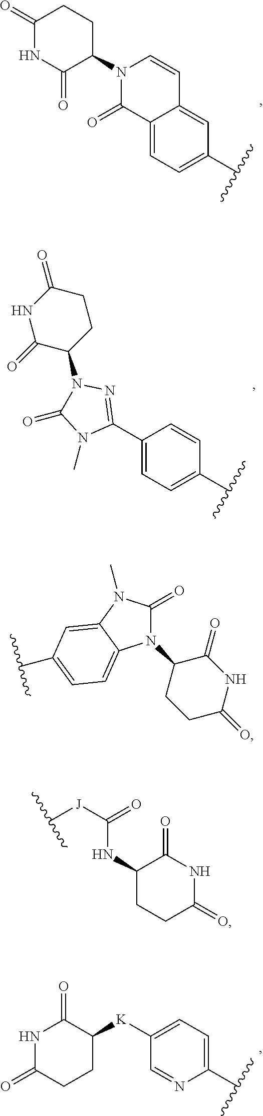

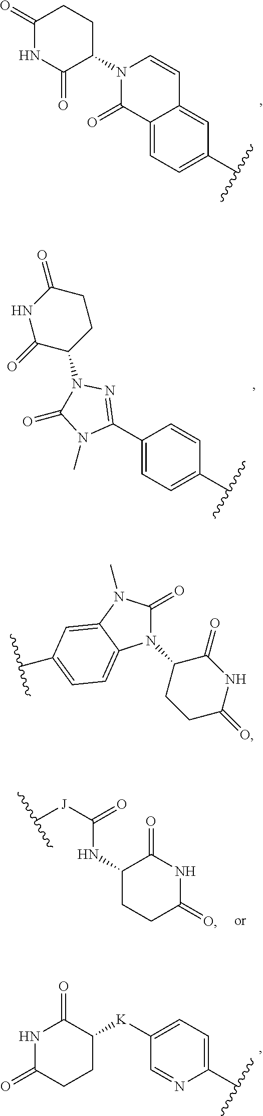

wherein each J is independently aryl or heteroaryl; and each K is independently absent, —CH₂—, —NH—, —NMe—, or —O—.

In certain embodiments of any of Formulas A-X or I-IV, Y is any of the following:

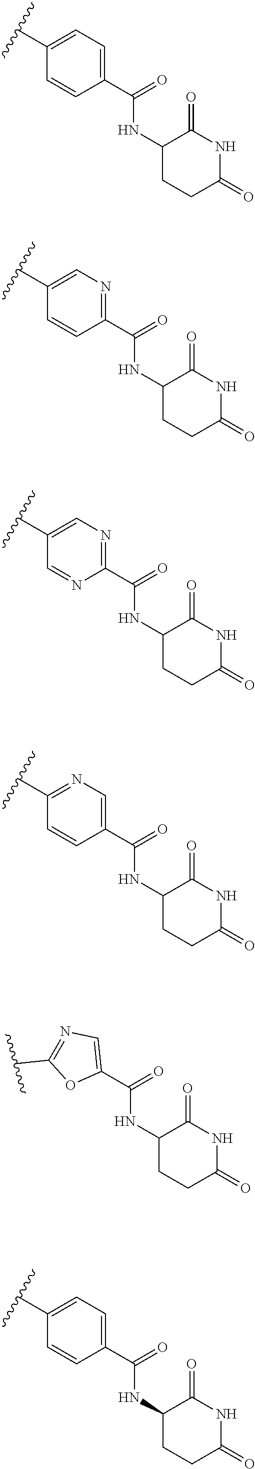 [SMILES: *c1ccc(C(=O)NC2CCC(=O)NC2=O)cc1 ; *c1ccc(C(=O)NC2CCC(=O)NC2=O)nc1 ; *c1cnc(C(=O)NC2CCC(=O)NC2=O)nc1 ; *c1ccc(C(=O)NC2CCC(=O)NC2=O)cn1 ; *c1ncc(C(=O)NC2CCC(=O)NC2=O)o1 ; *c1ccc(C(=O)N[C@@H]2CCC(=O)NC2=O)cc1]

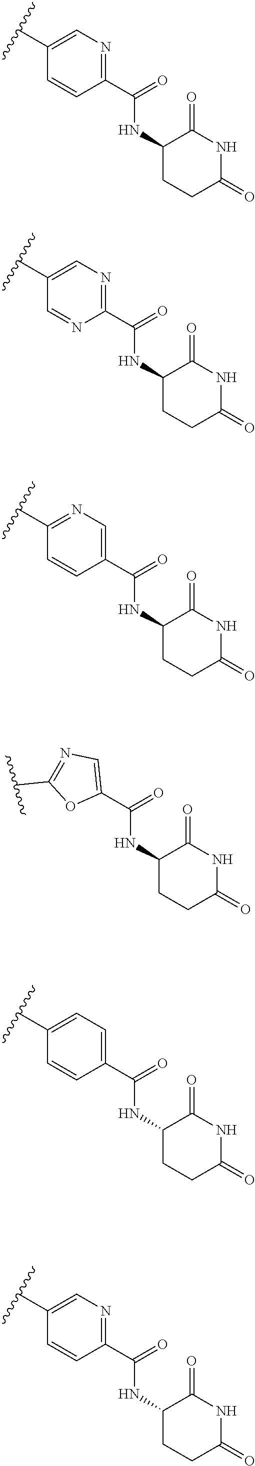 [SMILES: *c1ccc(C(=O)N[C@@H]2CCC(=O)NC2=O)nc1 ; *c1cnc(C(=O)N[C@@H]2CCC(=O)NC2=O)nc1 ; *c1ccc(C(=O)N[C@@H]2CCC(=O)NC2=O)cn1 ; *c1ncc(C(=O)N[C@@H]2CCC(=O)NC2=O)o1 ; *c1ccc(C(=O)N[C@H]2CCC(=O)NC2=O)cc1 ; *c1ccc(C(=O)N[C@H]2CCC(=O)NC2=O)nc1]

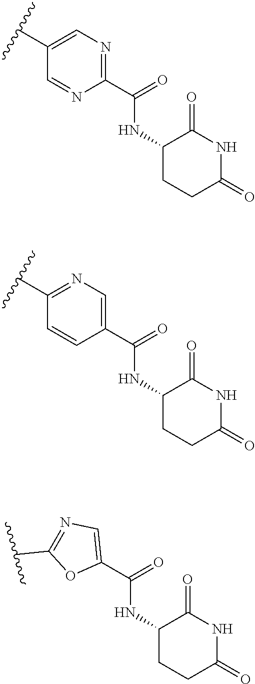 [SMILES: *c1cnc(C(=O)N[C@H]2CCC(=O)NC2=O)nc1 ; *c1ccc(C(=O)N[C@H]2CCC(=O)NC2=O)cn1 ; *c1ncc(C(=O)N[C@H]2CCC(=O)NC2=O)o1]

In certain embodiments of any of Formulas A-X or I-IV, Y is any of the following:

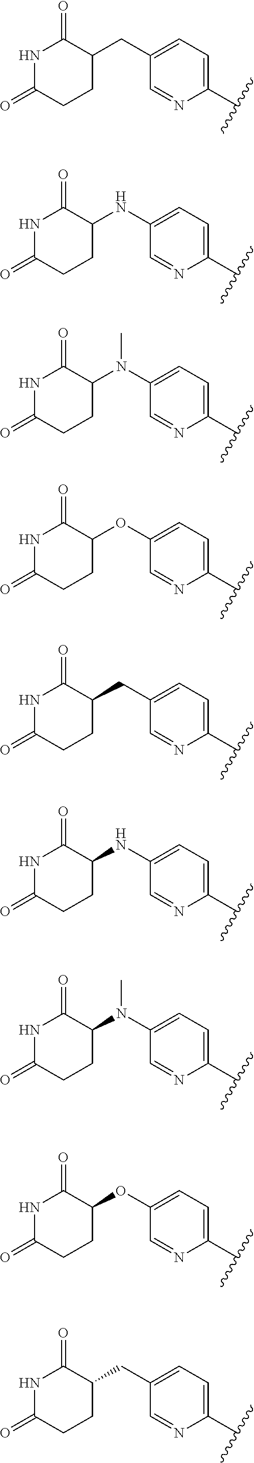

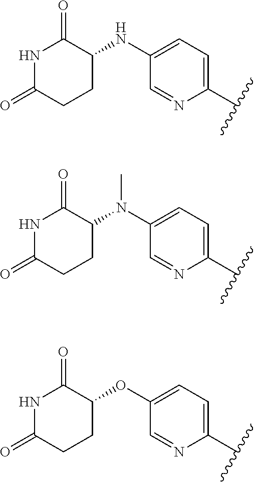

### General Synthetic Schemes

Compounds can be prepared or synthesized according to any technique deemed suitable by the person of skill in the art. In certain embodiments, compounds are prepared according to International Application No. PCT/US2019/56112, filed Oct. 14, 2019, incorporated by reference herein in its entirety. Exemplary synthetic schemes are described below.

### General Procedure 1: Amide Coupling

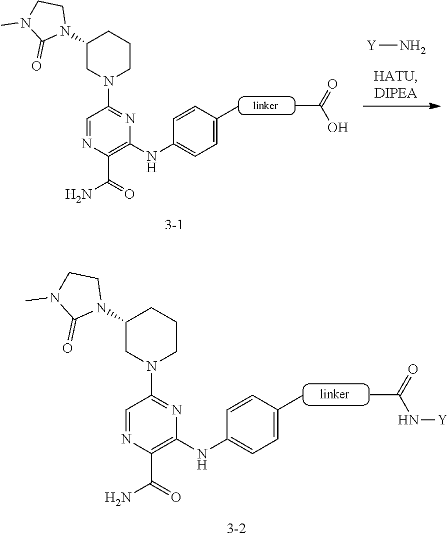

Intermediate (3-1), which can be generated by de-esterifying intermediate (1-6), is treated with amine, Y—NH₂, under coupling conditions to generate compounds of this disclosure (3-2), wherein the terminal linking group of L is an amide.

### General Procedure 2: Reductive Amination.

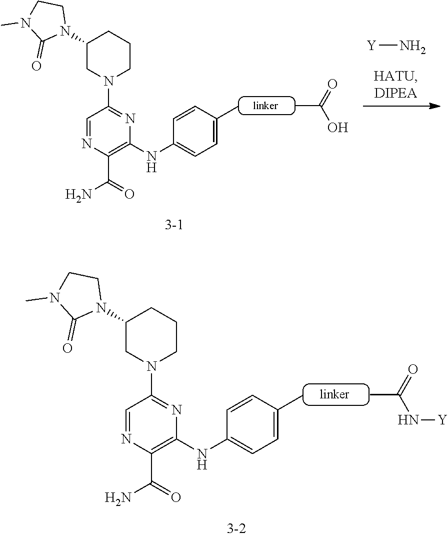

Intermediate (3-1), which can be generated by de-esterifying intermediate (1-6), is treated with amine, Y—NH₂, under coupling conditions to generate compounds of the present invention (3-2), wherein the terminal linking group of L is an amide.

General Procedure 3: Aryl fluoride displacement.

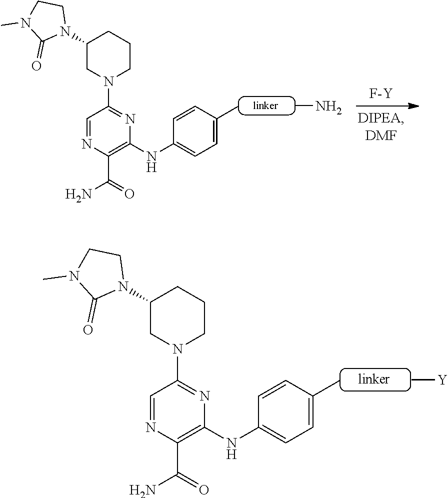

Intermediate (3-1), which can be generated by de-esterifying intermediate (1-6), is treated with any aryl fluoride, Y—F, under coupling conditions to generate compounds of the present invention (3-2), wherein the terminal linking group of L is an NH₂.

The abovementioned synthetic schemes can be used to synthesize the compounds in Table 1.

TABLE 1
Example compounds and/or pharmaceutically acceptable
salts thereof for use in the methods described herein.

| Com- | |
|---|---|
| pound | |
| Number | Structure |
| 1 | 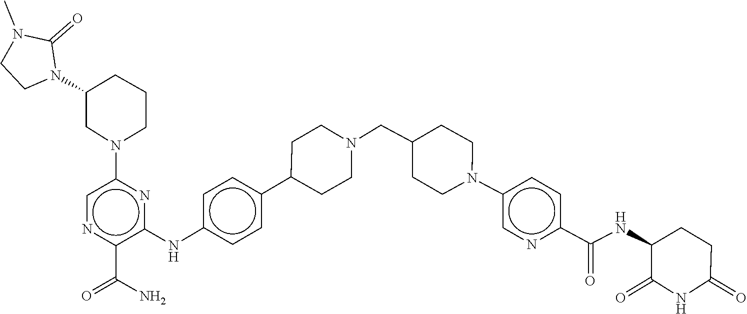 |
| 2 | 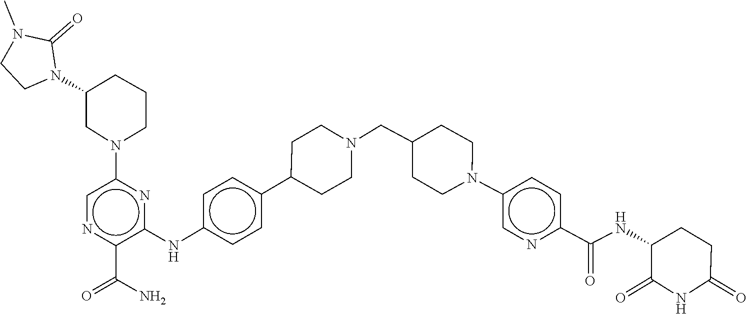 |
| 3 | 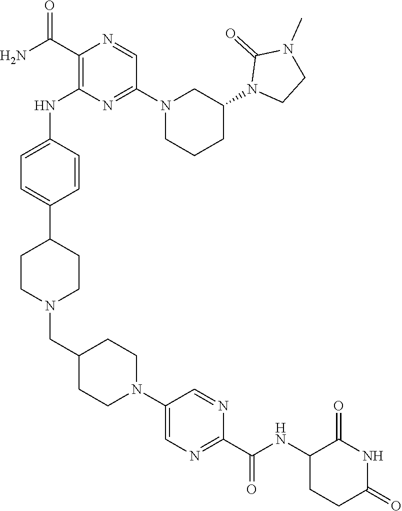 [SMILES: CN1CCN([C@@H]2CCCN(c3cnc(C(N)=O)c(Nc4ccc(C5CCN(CC6CCN(c7cnc(C(=O)NC8CCC(=O)NC8=O)nc7)CC6)CC5)cc4)n3)C2)C1=O] |
| 4 | 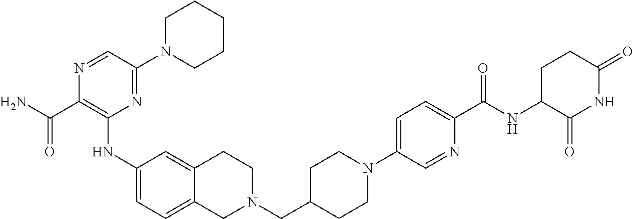 [SMILES: NC(=O)c1ncc(N2CCCCC2)nc1Nc1ccc2c(c1)CCN(CC1CCN(c3ccc(C(=O)NC4CCC(=O)NC4=O)nc3)CC1)C2] |
| 5 | 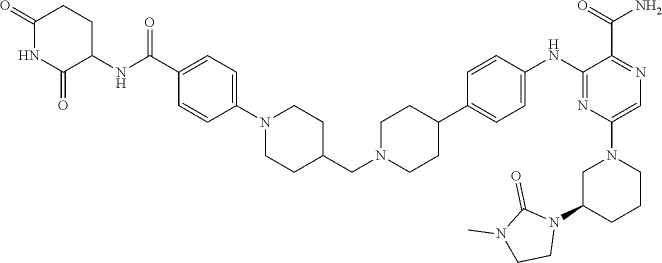 [SMILES: CN1CCN([C@@H]2CCCN(c3cnc(C(N)=O)c(Nc4ccc(C5CCN(CC6CCN(c7ccc(C(=O)NC8CCC(=O)NC8=O)cc7)CC6)CC5)cc4)n3)C2)C1=O] |
| 6 | 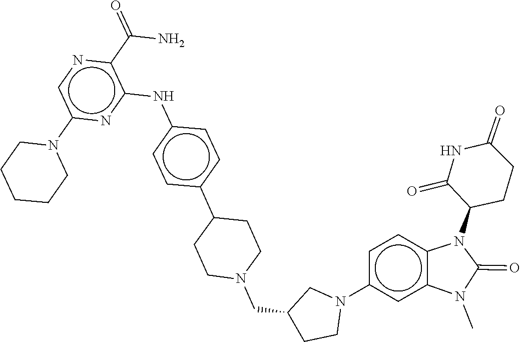 |
| 7 | 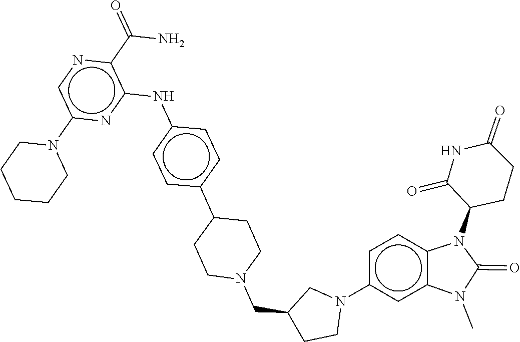 |
| 8 | 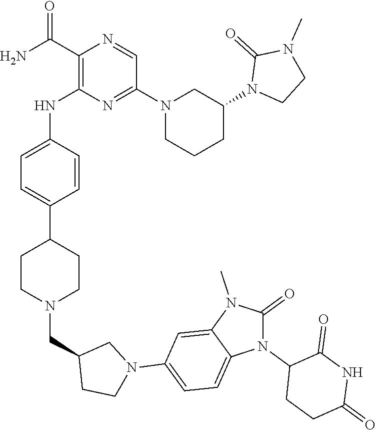 [SMILES: CN1CCN([C@@H]2CCCN(c3cnc(C(N)=O)c(Nc4ccc(C5CCN(C[C@@H]6CCN(c7ccc8c(c7)n(C)c(=O)n8C7CCC(=O)NC7=O)C6)CC5)cc4)n3)C2)C1=O] |
| 9 | 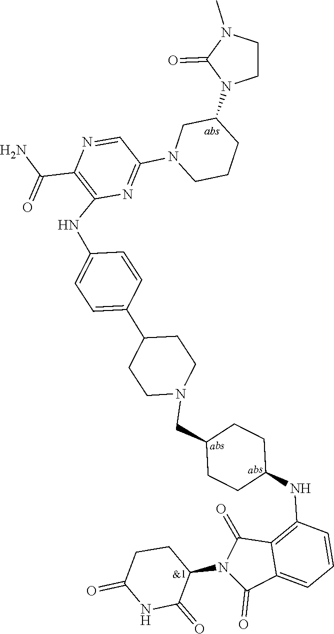 [SMILES: CN1CCN([C@@H]2CCCN(c3cnc(C(N)=O)c(Nc4ccc(C5CCN(C[C@H]6CC[C@@H](Nc7cccc8c7C(=O)N([C@@H]7CCC(=O)NC7=O)C8=O)CC6)CC5)cc4)n3)C2)C1=O] |
| 10 | 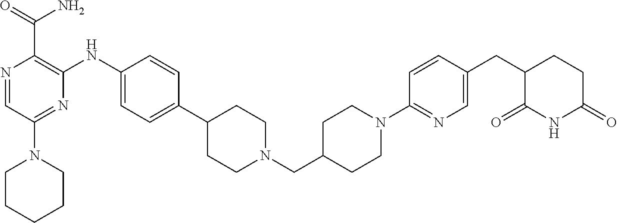 [SMILES: NC(=O)c1ncc(N2CCCCC2)nc1Nc1ccc(C2CCN(CC3CCN(c4ccc(CC5CCC(=O)NC5=O)cn4)CC3)CC2)cc1] |
| 11 | 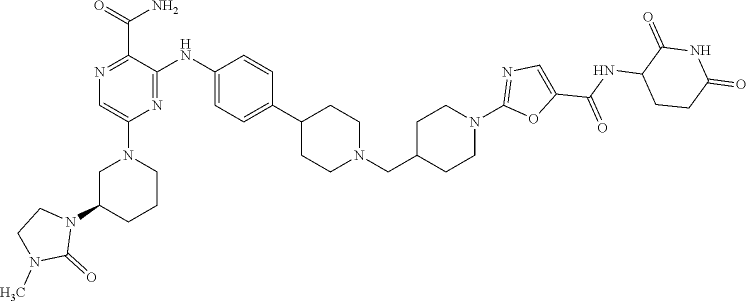 [SMILES: CN1CCN([C@@H]2CCCN(c3cnc(C(N)=O)c(Nc4ccc(C5CCN(CC6CCN(c7ncc(C(=O)NC8CCC(=O)NC8=O)o7)CC6)CC5)cc4)n3)C2)C1=O] |
| 12 | 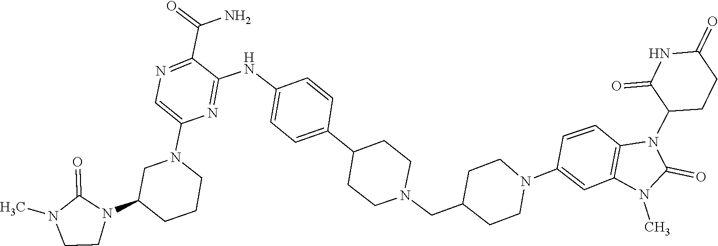 [SMILES: CN1CCN([C@@H]2CCCN(c3cnc(C(N)=O)c(Nc4ccc(C5CCN(CC6CCN(c7ccc8c(c7)n(C)c(=O)n8C7CCC(=O)NC7=O)CC6)CC5)cc4)n3)C2)C1=O] |
| 13 | 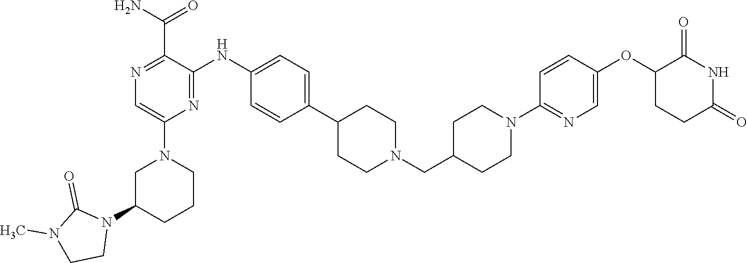 [SMILES: CN1CCN([C@@H]2CCCN(c3cnc(C(N)=O)c(Nc4ccc(C5CCN(CC6CCN(c7ccc(OC8CCC(=O)NC8=O)cn7)CC6)CC5)cc4)n3)C2)C1=O] |
| 14 | 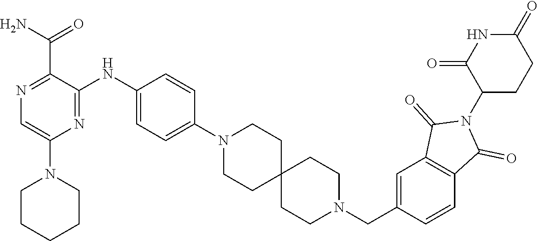 [SMILES: NC(=O)c1ncc(N2CCCCC2)nc1Nc1ccc(N2CCC3(CCN(Cc4ccc5c(c4)C(=O)N(C4CCC(=O)NC4=O)C5=O)CC3)CC2)cc1] |
| 15 | 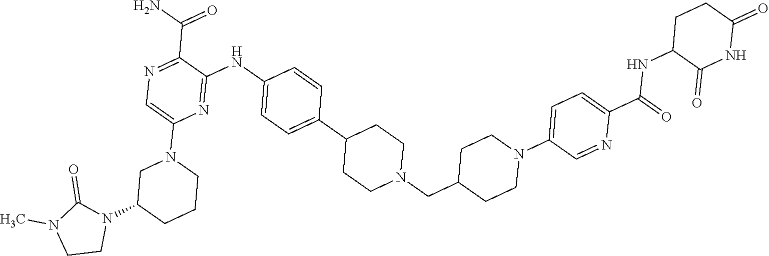 [SMILES: CN1CCN([C@H]2CCCN(c3cnc(C(N)=O)c(Nc4ccc(C5CCN(CC6CCN(c7ccc(C(=O)NC8CCC(=O)NC8=O)nc7)CC6)CC5)cc4)n3)C2)C1=O] |
| 16 | 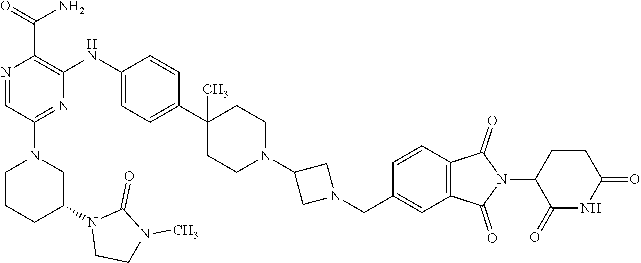 [SMILES: CN1CCN([C@@H]2CCCN(c3cnc(C(N)=O)c(Nc4ccc(C5(C)CCN(C6CN(Cc7ccc8c(c7)C(=O)N(C7CCC(=O)NC7=O)C8=O)C6)CC5)cc4)n3)C2)C1=O] |
| 17 | 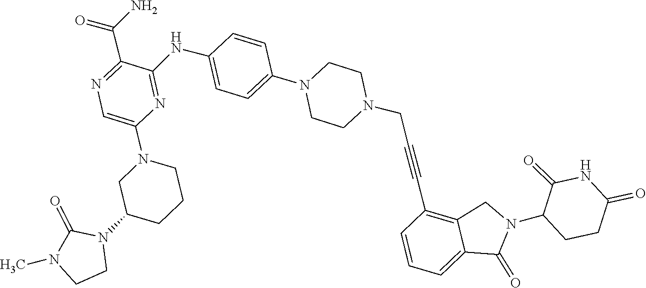 [SMILES: CN1CCN([C@H]2CCCN(c3cnc(C(N)=O)c(Nc4ccc(N5CCN(CC#Cc6cccc7c6CN(C6CCC(=O)NC6=O)C7=O)CC5)cc4)n3)C2)C1=O] |
| 18 | 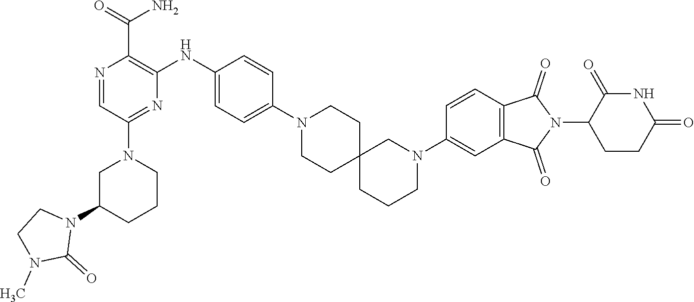 [SMILES: CN1CCN([C@@H]2CCCN(c3cnc(C(N)=O)c(Nc4ccc(N5CCC6(CCCN(c7ccc8c(c7)C(=O)N(C7CCC(=O)NC7=O)C8=O)C6)CC5)cc4)n3)C2)C1=O] |
| 19 | 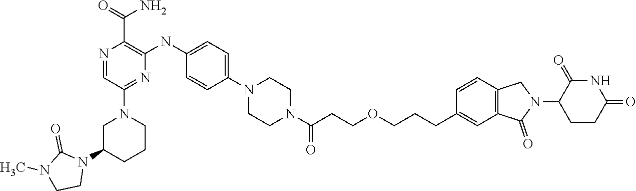 |
| 20 | 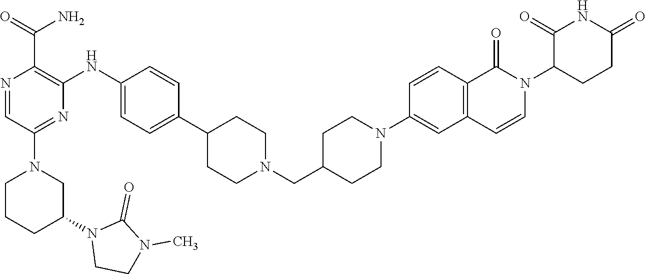 [SMILES: CN1CCN([C@@H]2CCCN(c3cnc(C(N)=O)c(Nc4ccc(C5CCN(CC6CCN(c7ccc8c(=O)n(C9CCC(=O)NC9=O)ccc8c7)CC6)CC5)cc4)n3)C2)C1=O] |
| 21 | 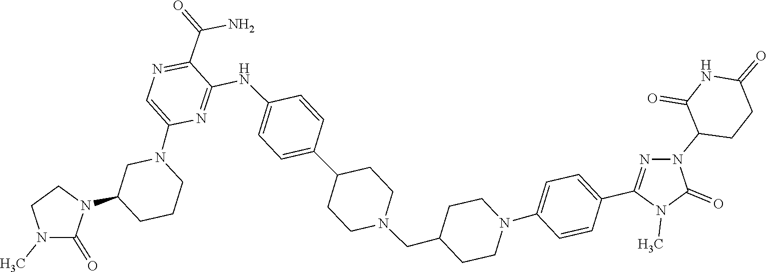 [SMILES: CN1CCN([C@@H]2CCCN(c3cnc(C(N)=O)c(Nc4ccc(C5CCN(CC6CCN(c7ccc(-c8nn(C9CCC(=O)NC9=O)c(=O)n8C)cc7)CC6)CC5)cc4)n3)C2)C1=O] |
| 22 | 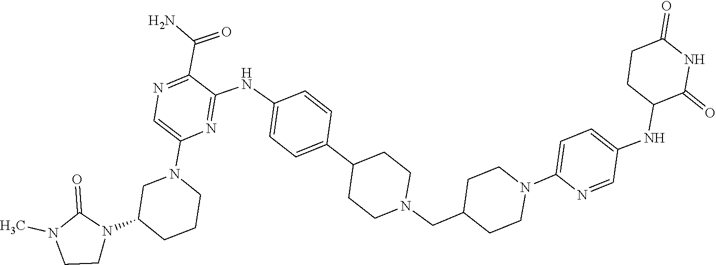 [SMILES: CN1CCN([C@H]2CCCN(c3cnc(C(N)=O)c(Nc4ccc(C5CCN(CC6CCN(c7ccc(NC8CCC(=O)NC8=O)cn7)CC6)CC5)cc4)n3)C2)C1=O] |

### Formulations and Administration

### Pharmaceutical Compositions

The compounds described herein can be formulated into pharmaceutical compositions that further comprise a pharmaceutically acceptable carrier, diluent, adjuvant, or vehicle. In one embodiment, this disclosure provides a pharmaceutical composition comprising a compound described above, and a pharmaceutically acceptable carrier, diluent, adjuvant, or vehicle. In one embodiment, this disclosure is a pharmaceutical composition comprising an effective amount of a compound of this disclosure or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to another embodiment, the description provides a composition comprising a compound herein or a pharmaceutically acceptable salt thereof and a pharmaceutically acceptable carrier, adjuvant, or vehicle. Pharmaceutical compositions of this description comprise a therapeutically effective amount of a compound of Formula A-X or I—IV wherein a “therapeutically effective amount” is an amount that is (a) effective to measurably degrade BTK (or reduce the amount of BTK) in a biological sample or in a patient; or (b) effective in treating and/or ameliorating a disease or disorder that is mediated by BTK.

The term “patient,” as used herein, means an animal, alternatively a mammal, and alternatively a human.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade,” means a measurable reduction in (a) BTK activity, between a sample comprising a compound of this description and a BTK and an equivalent sample comprising a BTK in the absence of said compound; or (b) the concentration of the BTK in a sample over time.

### Administration

The compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that maybe combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular condition, or disease, to be treated or prevented, additional therapeutic agents, which are normally administered to treat or prevent that condition, also may be present in the compositions of this disclosure. As used herein, additional therapeutic agents that are normally administered to treat or prevent a particular disease, or condition, are known as “appropriate for the disease, or condition, being treated.”

For example, chemotherapeutic agents or other anti-proliferative agents may be combined with the compounds of this disclosure to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxetan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy—Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of this disclosure (or a pharmaceutically acceptable salt thereof) is administered to a patient in need thereof.

Other examples of agents with which the compounds or inhibitors of this disclosure also may be combined include, without limitation, treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., AvoneX® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### EXAMPLES

Additional embodiments are disclosed in further detail in the following examples, which are not in any way intended to limit the scope of the claims.

### Example 1

### General Procedure 1: Amide Coupling

A mixture of amine (0.03 mmol), acid (0.03 mmol), HATU (0.04 mmol), DIPEA (0.15 mmol), and DMF was allowed to stir at room temperature for 30 minutes. The mixture was purified by HPLC (H₂O/MeCN with 0.1% TFA) to afford the amide product. Compound 21 was prepared according to procedure 1.

### General Procedure 2: Reductive Amination

A mixture of amine TFA salt (0.07 mmol), aldehyde (0.1 mmol), triethylamine (0.28 mmol), and DCE were allowed to stir at room temperature for 10 minutes. NaBH(OAc)₃ (0.14 mmol) was added and the mixture was allowed to stir at room temperature for 2 h. The mixture was filtered through celite, washed with CH₂Cl₂, concentrated, and purified by HPLC (H₂O/MeCN with 0.1% TFA) to afford the amine product. Compounds 6, 7, and 199 were prepared according to procedure 2.

### General Procedure 3: Aryl Fluoride Displacement

A mixture of amine (0.22 mmol), aryl fluoride (0.22 mmol), DIPEA (0.88 mmol), and DMF (1 mL) was allowed to stir at 90° C. for 16 h. The mixture was purified by HPLC (H₂O/MeCN with 0.1% TFA) to afford the desired product. Compound 20 was prepared according to procedure 3.

Compounds 10-22 are prepared according to PCT/US2019/56112, filed Oct. 14, 2019, which is hereby incorporated by reference in its entirety.

### Example 2

Prepared according to General Procedure 2:

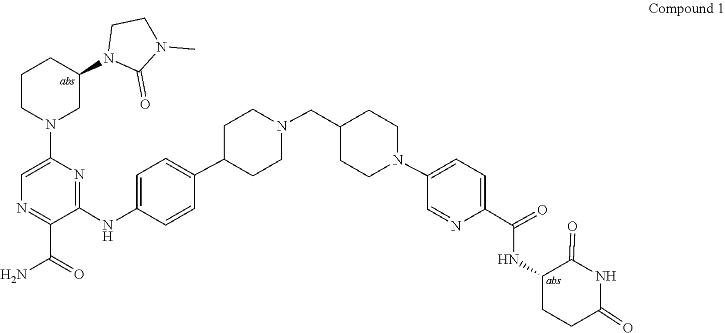 [SMILES: CN1CCN([C@@H]2CCCN(c3cnc(C(N)=O)c(Nc4ccc(C5CCN(CC6CCN(c7ccc(C(=O)N[C@H]8CCC(=O)NC8=O)nc7)CC6)CC5)cc4)n3)C2)C1=O]

¹H NMR (500 MHz, DMSO-d₆) δ 11.19 (s, 1H), 10.85 (s, 1H), 8.71 (d, J=8.2 Hz, 1H), 8.32 (d, J=2.9 Hz, 1H), 7.85 (d, J=8.8 Hz, 1H), 7.76 (s, 1H), 7.67 (s, 1H), 7.51 (d, J=8.1 Hz, 2H), 7.42 (dd, J=8.9, 2.9 Hz, 1H), 7.34 (s, 1H), 7.18 (d, J=8.2 Hz, 2H), 4.75 (ddd, J=13.2, 8.2, 5.4 Hz, 1H), 4.34 (dd, J=39.7, 12.8 Hz, 2H), 3.96 (d, J 12.5 Hz, 2H), 3.62 (d, J=11.0 Hz, 1H), 3.28 (dd, J=14.4, 7.4 Hz, 5H), 3.11-2.76 (m, 8H), 2.73 (s, 3H), 2.19 (dd, J=10.7, 5.3 Hz, 3H), 2.08-1.93 (m, 3H), 1.89-1.71 (m, 8H), 1.59 (d, J=29.1 Hz, 4H), 1.23 (d, J=14.3 Hz, 5H). LCMS: C₄₂H₅₄N₁₂O₅ requires: 806, found: m/z=807 [M+H]⁺.

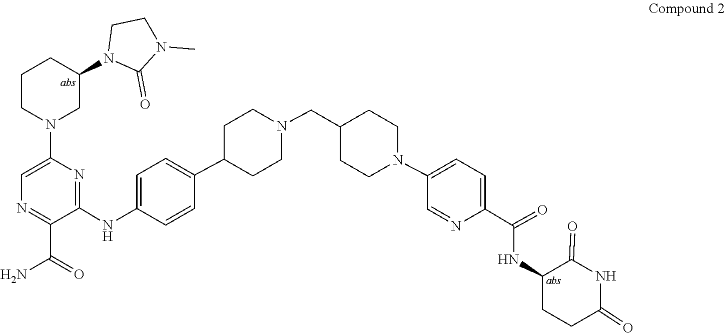 [SMILES: CN1CCN([C@@H]2CCCN(c3cnc(C(N)=O)c(Nc4ccc(C5CCN(CC6CCN(c7ccc(C(=O)N[C@@H]8CCC(=O)NC8=O)nc7)CC6)CC5)cc4)n3)C2)C1=O]

¹H NMR (500 MHz, DMSO-d₆) δ 11.30 (s, 1H), 10.86 (s, 1H), 8.71 (d, J=8.1 Hz, 1H), 8.36 (s, 1H), 7.88 (d, J=8.7 Hz, 1H), 7.79 (s, 1H), 7.69 (s, 1H), 7.58 (d, J=7.9 Hz, 2H), 7.46 (d, J 8.8 Hz, 1H), 7.36 (s, 1H), 7.19 (d, J 8.2 Hz, 2H), 4.75 (ddd, J=13.1, 8.2, 5.4 Hz, 1H), 4.42-4.25 (m, 2H), 4.01 (d, J=12.9 Hz, 2H), 3.65 (dd, J=13.8, 7.9 Hz, 3H), 3.27 (t, J=8.3 Hz, 3H), 3.17-2.87 (m, 6H), 2.86-2.70 (m, 5H), 2.27-2.10 (m, 2H), 2.11-1.69 (m, 12H), 1.68-1.47 (m, 2H), 1.30 (d, J=52.1 Hz, 3H). LCMS: C₄₂H₅₄N₁₂O₅ requires: 806, found: m/z=807 [M+H]⁺.

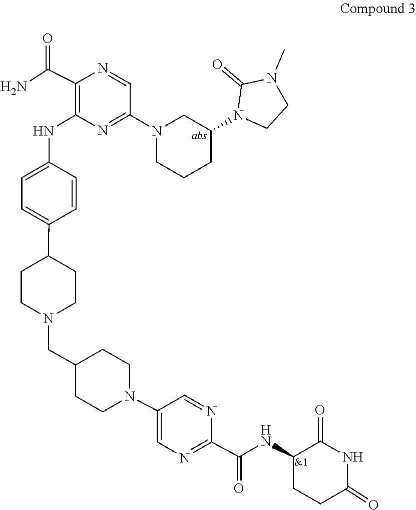 [SMILES: CN1CCN([C@@H]2CCCN(c3cnc(C(N)=O)c(Nc4ccc(C5CCN(CC6CCN(c7cnc(C(=O)N[C@@H]8CCC(=O)NC8=O)nc7)CC6)CC5)cc4)n3)C2)C1=O]

Prepared by a procedure analogous to 3-((4-(1-((1-(6-(((S)-2,6-dioxopiperidin-3-yl)carb amoyl)pyridin-3-yl)piperidin-4-yl)methyl)piperidin-4-yl)phenyl)amino)-5-((R)-3-(3-methyl-2-oxoimidazolidin-1-yl)piperidin-1-yl)pyrazine-2-carboxamide but with methyl 5-bromopyrimidine-2-carboxylate as a starting material. Obtained 5-(4-((4-(4-((3-carbamoyl-6-((R)-3-(3-methyl-2-oxoimidazolidin-1-yl)piperidin-1-yl)pyrazin-2-yl)amino)phenyl)piperidin-1-yl)methyl)piperidin-1-yl)-N—((RS)-2,6-dioxopiperidin-3-yl)pyrimidine-2-carboxamide (33 mg). ¹H NMR (500 MHz, DMSO-d6) δ 11.35-11.26 (m, 1H), 10.88 (s, 1H), 8.91 (s, 1H), 8.84 (d, J=8.2 Hz, 1H), 8.61 (s, 2H), 7.80 (s, 1H), 7.69 (s, 1H), 7.58 (d, J=8.0 Hz, 2H), 7.36 (s, 1H), 7.20 (d, J=8.3 Hz, 2H), 4.76 (ddd, J=13.0, 8.3, 5.3 Hz, 1H), 4.38-4.27 (m, 2H), 4.09 (d, J=12.8 Hz, 2H), 3.68-3.61 (m, 3H), 3.35 (dt, J=14.2, 7.7 Hz, 2H), 3.27 (t, J=8.0 Hz, 2H), 3.14-2.92 (m, 8H), 2.87-2.75 (m, 2H), 2.73 (s, 3H), 2.60-2.44 (m, 1H), 2.26-2.13 (m, 2H), 2.10-1.72 (m, 10H), 1.62-1.53 (m, 1H), 1.34 (q, J=11.4 Hz, 2H). LCMS: C₄₁H₅₃N₁₃O₅ requires 807, found: m/z=808 [M+H]⁺.

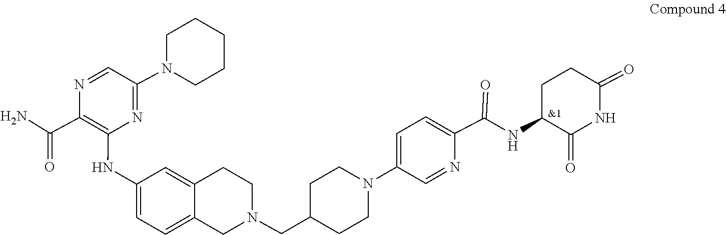 [SMILES: NC(=O)c1ncc(N2CCCCC2)nc1Nc1ccc2c(c1)CCN(CC1CCN(c3ccc(C(=O)N[C@H]4CCC(=O)NC4=O)nc3)CC1)C2]

¹H NMR (500 MHz, Acetonitrile-d₃) δ 11.26 (s, 1H), 9.59 (s, 1H), 8.78 (s, 1H), 8.35-8.26 (m, 2H), 7.95 (d, J=8.8 Hz, 1H), 7.70 (s, 1H), 7.61 (s, 1H), 7.52 (d, J=8.5 Hz, 1H), 7.43 (s, 1H), 7.37 (dd, J=8.9, 2.8 Hz, 1H), 7.15 (d, J=8.4 Hz, 1H), 5.84 (s, 1H), 4.81-4.70 (m, 1H), 4.59 (d, J=15.2 Hz, 1H), 4.18 (d, J=15.1 Hz, 1H), 3.98 (d, J=13.1 Hz, 2H), 3.73 (t, J=5.4 Hz, 5H), 3.44-3.22 (m, 2H), 3.13 (d, J=20.2 Hz, 3H), 2.97 (t, J=12.4 Hz, 2H), 2.84-2.65 (m, 3H), 2.27-2.07 (m, 1H), 1.80-1.62 (m, 7H), 1.45 (q, J=11.2, 10.3 Hz, 2H). LCMS: C₃₆H₄₄N₁₀O₄ requires 680, found: m/z=681 [M+H]⁺.

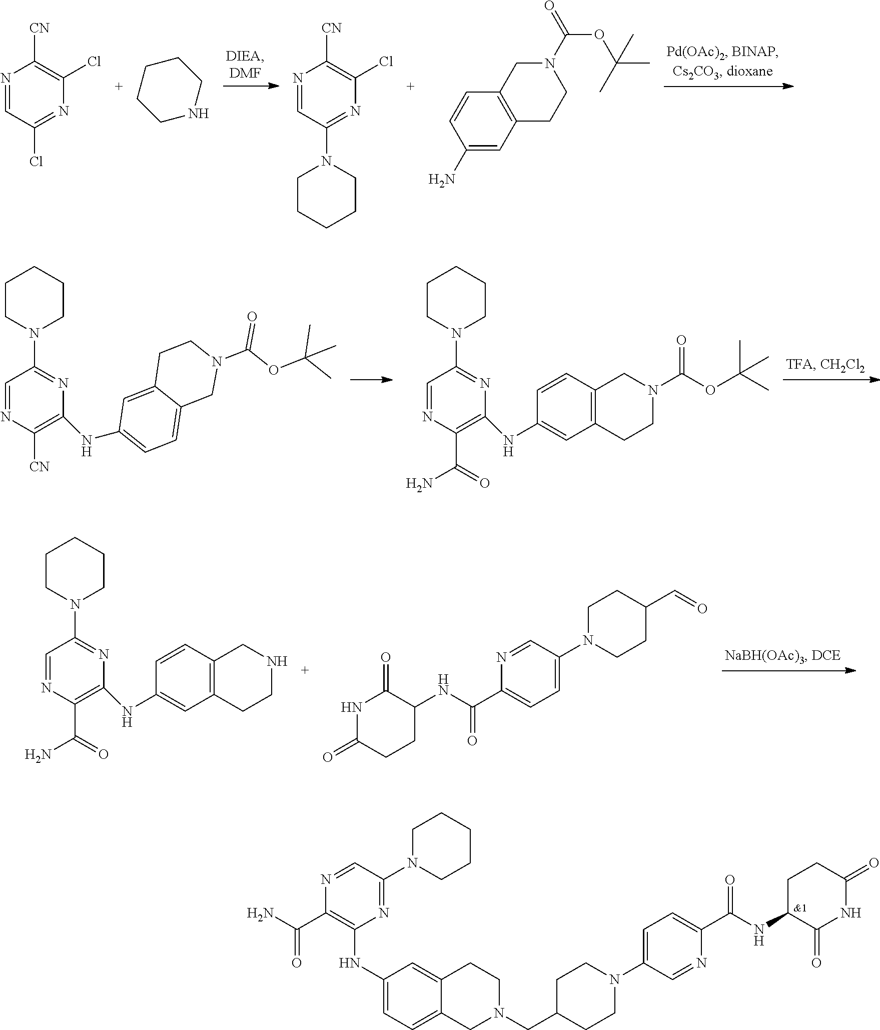

A mixture of piperidine (2.55 mL, 2194 mg, 25.76 mmol), 3,5-dichloropyrazine-2-carbonitrile (4075 mg, 23.42 mmol), N,N-diisopropylethylamine (8.16 mL, 46.84 mmol) and DMF (100 mL) was allowed to stir at room temperature for 2 h. EtOAc and H₂O were added. The organic layer was dried with MgSO₄, filtered, concentrated and purified by MPLC (0-100% EtOAc in hexanes) to afford 3-chloro-5-(piperidin-1-yl)pyrazine-2-carbonitrile (4.08 g, 78%). LCMS: C₁₀H₁₁ClN₄ requires 222, found: m/z=223 [M+H]⁺.

A mixture of tert-butyl 6-amino-3,4-dihydro-1H-isoquinoline-2-carboxylate (1398.42 mg, 5.63 mmol), 3-chloro-5-(piperidin-1-yl)pyrazine-2-carbonitrile (1254 mg, 5.63 mmol), (acetyloxy)palladio acetate (252.86 mg, 1.13 mmol), [2′-(diphenylphosphanyl)-[1,1′-binaphthalen]-2-yl]diphenylphosphane (701.32 mg, 1.13 mmol) and dicaesium carbonate (5504 mg, 16.89 mmol) was degassed and backfilled with N₂ 5 times. Dioxane (30 mL) was added and the mixture was allowed to stir at 100° C. for 90 min. The mixture was filtered through celite washing with MeOH/EtOAc, concentrated and purified by MPLC (0-100% EtOAc in CH₂Cl₂) to afford tert-butyl 6-((3-cyano-6-(piperidin-1-yl)pyrazin-2-yl)amino)-3,4-dihydroisoquinoline-2 (1H)-carboxylate (1.83 g, 74.8%). LCMS: C₂₄H₃₀N₆O₂ requires 434, found: m/z=435 [M+H]⁺.

H₂O₂ 30% aqueous solution (7.11 mL) was added to a mixture of dicaesium carbonate (1372 mg, 4.21 mmol), DMSO (2 mL), MeOH (40 mL) and tert-butyl 6-((3-cyano-6-(piperidin-1-yl)pyrazin-2-yl)amino)-3,4-dihydroisoquinoline-2 (1H)-carboxylate (1830 mg, 4.21 mmol). The mixture was allowed to stir at room temperature for 30 min. The mixture was concentrated. EtOAc was added and the organic phase was washed with H₂O and brine. The organic layer was dried with MgSO₄, filtered, concentrated and purified by MPLC (0-10% MeOH in CH₂Cl₂) to afford tert-butyl 6-((3-carbamoyl-6-(piperidin-1-yl)pyrazin-2-yl)amino)-3,4-dihydroisoquinoline-2 (1H)-carboxylate (1.647 g, 86.4%). LCMS: C₂₄H₃₂N₆O₃ requires 452, found: m/z=453 [M+H]⁺.

A mixture of tert-butyl 6-((3-carb amoyl-6-(piperidin-1-yl)pyrazin-2-yl)amino)-3,4-dihydroisoquinoline-2 (1H)-carboxylate (1647 mg, 3.64 mmol), CH₂Cl₂ (30 mL) and TFA (6 mL) was allowed to stir at room temperature for 2 h. The volatiles were removed. The mixture was filtered through a NaHCO₃ cartridge, concentrated and purified by reverse phase MPLC (5-90% MeCN in H₂O) to afford 5-(piperidin-1-yl)-3-((1,2,3,4-tetrahydroisoquinolin-6-yl)amino)pyrazine-2-carboxamide (1.28 g, 99.8%). LCMS: C₁₉H₂₄N₆O requires 352, found: m/z=353 [M+H]⁺.

Sodium bis(acetyloxy)boranuidyl acetate (32.47 mg, 0.15 mmol) was added to a mixture of triethylamine (0.03 mL, 0.20 mmol), rac-N-[(3R)-2,6-dioxopiperidin-3-yl]-5-(4-formylpiperidin-1-yl)pyridine-2-carboxamide (21.11 mg, 0.06 mmol), 5-(piperidin-1-yl)-3-(1,2,3,4-tetrahydroisoquinolin-6-ylamino)pyrazine-2-carboxamide (18.00 mg, 0.05 mmol) and DCE (1 mL). The mixture was filtered through celite, washing with CH₂Cl₂/EtOAc, concentrated and purified by HPLC (5-95% MeCN in H₂O with 0.1% TFA) to afford rac-3-[(2-{[1-(6-{[(3R)-2,6-dioxopiperidin-3-yl]carbamoyl}pyridin-3-yl)piperidin-4-yl]methyl}-3,4-dihydro-1H-isoquinolin-6-yl)amino]-5-(piperidin-1-yl)pyrazine-2-carboxamide (0.0130 g, 37.4%).

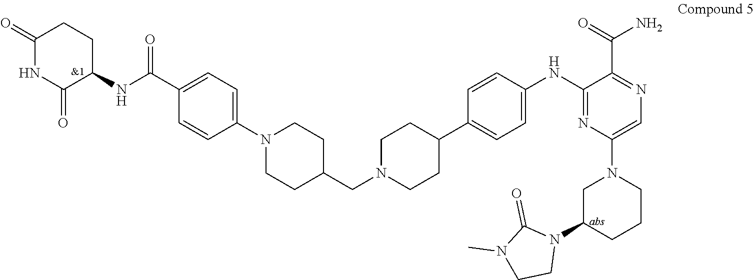 [SMILES: CN1CCN([C@@H]2CCCN(c3cnc(C(N)=O)c(Nc4ccc(C5CCN(CC6CCN(c7ccc(C(=O)N[C@@H]8CCC(=O)NC8=O)cc7)CC6)CC5)cc4)n3)C2)C1=O]

Prepared by a procedure analogous to 3-((4-(1-((1-(6-(((S)-2,6-dioxopiperidin-3-yl)carb amoyl)pyridin-3-yl)piperidin-4-yl)methyl)piperidin-4-yl)phenyl)amino)-5-((R)-3-(3-methyl-2-oxoimidazolidin-1-yl)piperidin-1-yl)pyrazine-2-carboxamide but with methyl 4-bromobenzoate as a starting material. Obtained 3-((4-(1-((1-(4-(((RS)-2,6-dioxopiperidin-3-yl)carb amoyl)phenyl)piperidin-4-yl)methyl)piperidin-4-yl)phenyl)amino)-5-((R)-3-(3-methyl-2-oxoimidazolidin-1-yl)piperidin-1-yl)pyrazine-2-carboxamide (13 mg). ¹H NMR (500 MHz, DMSO-d₆) δ 11.20 (s, 1H), 10.87 (s, 1H), 7.76 (d, J=2.9 Hz, 1H), 7.67 (s, 1H), 7.51 (d, J=8.2 Hz, 2H), 7.34 (s, 2H), 7.17 (d, J=8.2 Hz, 2H), 6.95 (d, J=8.3 Hz, 2H), 5.01 (s, 1H), 4.72 (s, OH), 4.37 (d, J=12.3 Hz, 1H), 4.29 (d, J=13.3 Hz, 1H), 3.82 (d, J=12.3 Hz, 2H), 3.63 (ddt, J=10.9, 8.4, 4.2 Hz, 1H), 3.40-3.25 (m, 3H), 3.03 (dd, J=23.3, 11.6 Hz, 1H), 2.98-2.89 (m, 4H), 2.73 (s, 9H), 2.48-2.33 (m, 3H), 2.21-2.17 (m, 2H), 2.09 (s, 1H), 1.97 (d, J=13.5 Hz, 3H), 1.87-1.72 (m, 7H), 1.65-1.52 (m, 3H), 1.20 (t, J=12.3 Hz, 2H), 0.08 (s, 1H). LCMS: C₄₃H₅₅N₁₁₀₅ requires 805, found: m/z=806 [M+H]⁺.

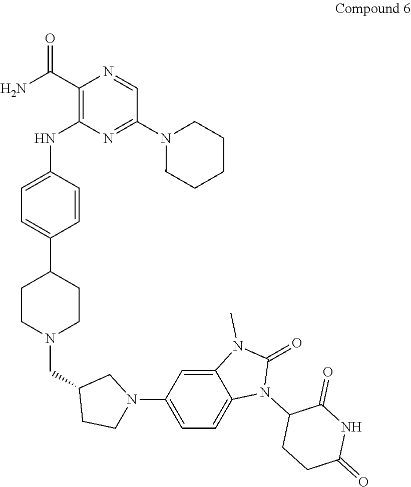 [SMILES: Cn1c(=O)n(C2CCC(=O)NC2=O)c2ccc(N3CC[C@H](CN4CCC(c5ccc(Nc6nc(N7CCCCC7)cnc6C(N)=O)cc5)CC4)C3)cc21]

¹H NMR (500 MHz, acetonitrile-d₃) δ 11.17 (s, 1H), 8.91 (s, 1H), 7.65 (d, J=8.2 Hz, 2H), 7.58 (s, 1H), 7.41 (s, 1H), 7.24 (d, J=8.2 Hz, 2H), 6.84 (d, J=8.6 Hz, 1H), 6.41 (s, 1H), 6.33 (d, J=8.7 Hz, 1H), 5.79 (s, 1H), 5.14 (dd, J=12.9, 5.3 Hz, 1H), 3.81-3.65 (m, 5H), 3.61 (t, J=8.3 Hz, 1H), 3.49-3.39 (m, 2H), 3.38-3.31 (m, 4H), 3.27-3.20 (m, 2H), 3.20-3.12 (m, 1H), 3.10-2.96 (m, 2H), 2.95-2.80 (m, 3H), 2.80-2.67 (m, 2H), 2.54-2.01 (m, 6H), 1.92-1.80 (m, 1H), 1.78-1.70 (m, 2H), 1.70-1.65 (m, 4H). LCMS: C₃₉H₄₈N₁₀O₄ requires 720, found: m/z=721 [M+H]⁺.

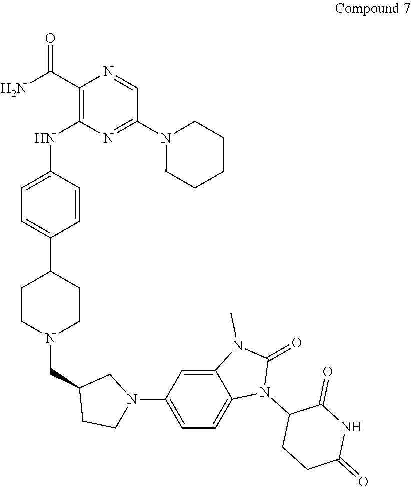 [SMILES: Cn1c(=O)n(C2CCC(=O)NC2=O)c2ccc(N3CC[C@@H](CN4CCC(c5ccc(Nc6nc(N7CCCCC7)cnc6C(N)=O)cc5)CC4)C3)cc21]

¹H NMR (500 MHz, acetonitrile-d₃) δ 11.18 (s, 1H), 9.77 (s, 1H), 8.91 (s, 1H), 7.65 (d, J=8.2 Hz, 2H), 7.58 (s, 1H), 7.41 (s, 1H), 7.24 (d, J=8.2 Hz, 2H), 6.85 (d, J=8.5 Hz, 1H), 6.41 (s, 1H), 6.33 (d, J=8.5 Hz, 1H), 5.80 (s, 1H), 5.15 (dd, J=12.8, 5.4 Hz, 1H), 3.78-3.70 (m, 5H), 3.61 (t, J=8.5 Hz, 1H), 3.49-3.39 (m, 2H), 3.38-3.31 (m, 4H), 3.28-3.20 (m, 2H), 3.20-3.13 (m, 1H), 3.12-2.96 (m, 2H), 2.92-2.84 (m, 3H), 2.80-2.71 (m, 2H), 2.57-2.03 (m, 6H), 1.92-1.79 (m, 1H), 1.80-1.71 (m, 2H), 1.71-1.65 (m, 4H). LCMS: C₃₉H₄₈N₁₀O₄ requires 720, found: m/z=721 [M+H]⁺.

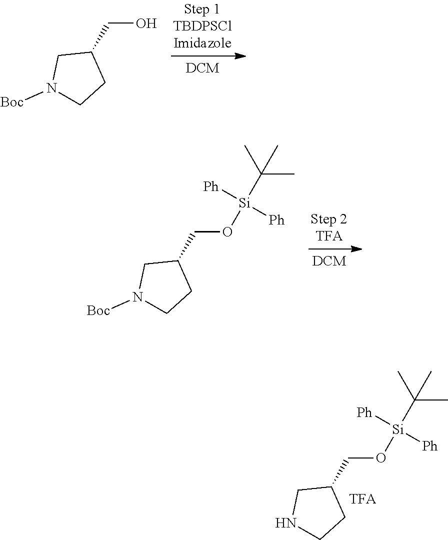 [SMILES: CC(C)(C)OC(=O)N1CC[C@@H](CO)C1 ; CC(C)(C)OC(=O)N1CC[C@@H](CO[Si](c2ccccc2)(c2ccccc2)C(C)(C)C)C1 ; CC(C)(C)[Si](OC[C@@H]1CCNC1)(c1ccccc1)c1ccccc1]

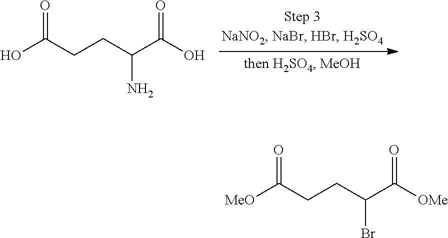 [SMILES: NC(CCC(=O)O)C(=O)O ; COC(=O)CCC(Br)C(=O)OC]

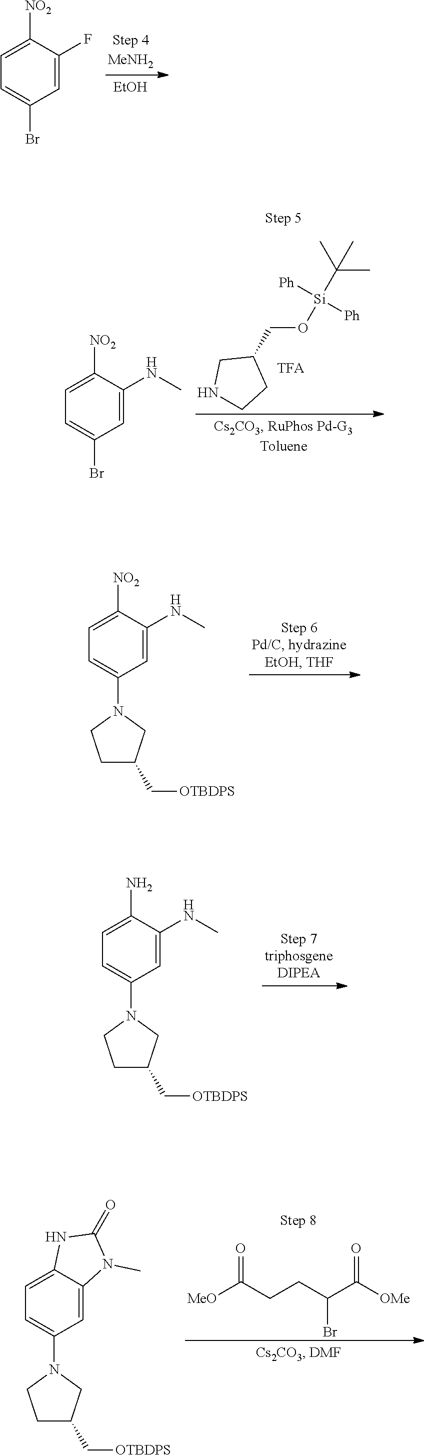

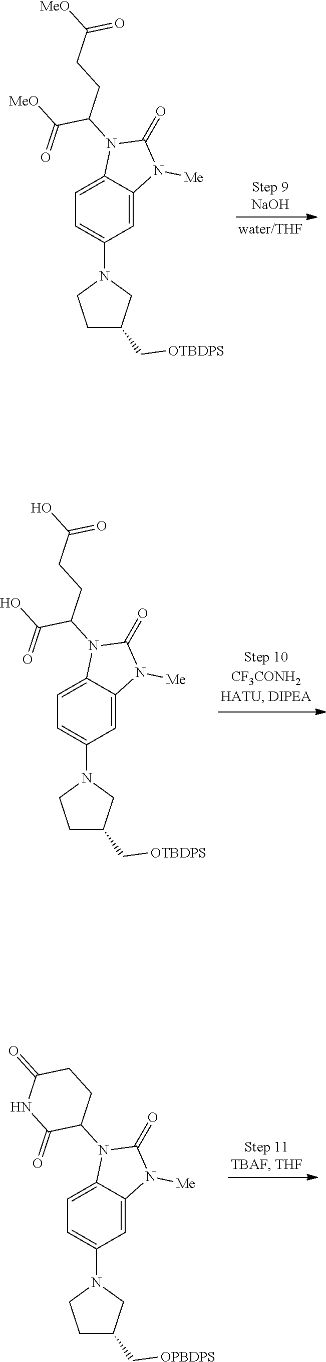

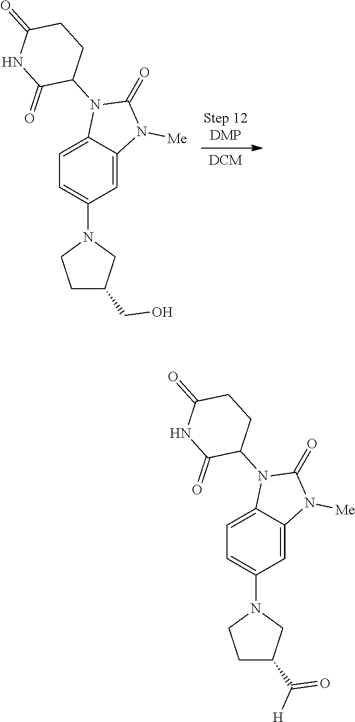 [SMILES: Cn1c(=O)n(C2CCC(=O)NC2=O)c2ccc(N3CC[C@@H](CO)C3)cc21 ; Cn1c(=O)n(C2CCC(=O)NC2=O)c2ccc(N3CC[C@@H](C=O)C3)cc21]

### Step 1: tert-butyl (3R)-3-[[tert-butyl(diphenyl)silylloxymethyl]pyrrolidine-1-carboxylate

TBDPSCl (32.3 mL, 124 mmol) was added to a mixture of tert-butyl (3R)-3-(hydroxymethyl)pyrrolidine-1-carboxylate (25.0 g, 124 mmol) and imidazole (10.1 g, 149 mmol) in DCM (500 mL) at 0° C. under nitrogen. The mixture was stirred at 23° C. for 16 h and diluted with water (300 mL). The organic phase was washed with water (100 mL), brine (3×100 mL), dried (Na₂SO₄), filtered, and concentrated to provide the title product as an oil (54.0 g, 99%). m/z: ES⁺[M−C₆H₅−tBu+H]⁺=306.2, LCMS (A05); t_{R}=2.47 min.

¹H NMR (500 MHz, CDCl₃) δ 7.67-7.60 (m, 4H), 7.46-7.34 (m, 6H), 3.64-3.55 (m, 2H), 3.45-3.37 (m, 1H), 3.37-3.22 (m, 1H), 3.17-3.07 (m, 1H), 2.42 (m, 1H), 1.97-1.86 (m, 1H), 1.74-1.62 (m, 1H), 1.60 (s, 1H), 1.46 (s, 9H), 1.08-1.02 (m, 9H);

### Step 2: tert-butyl-diphenyl-[[(3R)-pyrrolidin-3-yl]methoxy]silane 2,2,2-trifluoroacetic acid

TFA (50 mL) was added to a mixture of tert-butyl (3R)-3-[[tert-butyl(diphenyl)silyl]oxymethyl]pyrrolidine-1-carboxylate, (54.0 g, 123 mmol) in DCM (200 mL) at 23° C. under nitrogen. The mixture was stirred at 23° C. for 1.5 h and concentrated. The residue was diluted with PhMe (150 mL) and concentrated (process repeated twice) to provide the title compound as an oil (55.7 g, quant.). m/z: ES⁺[M+H-TFA]⁺=340.3, LCMS (A05); t_{R}=2.32 min. ¹H NMR (500 MHz, CDCl₃) δ 8.82 (s, 2H), 7.65-7.57 (m, 4H), 7.52-7.40 (m, 6H), 3.65 (d, J=6.4 Hz, 2H), 3.35-3.27 (m, 1H), 3.24-3.10 (m, 2H), 3.01-2.91 (m, 1H), 2.58-2.52 (m, 1H), 2.04-1.93 (m, 1H), 1.74-1.63 (m, 1H), 1.01 (s, 9H);

### Step 3: dimethyl 2-bromopentanedioate

A solution of NaNO₂ (25.5 g, 370 mmol) in water (50 mL) was added to a mixture of (2S′)-2-aminopentanedioic acid (30 g, 204 mmol), NaBr (73.2 g, 711 mol) and HBr (50 mL, 48% in water), in water (100 mL) at 0° C. (keeping the internal temperature below 10° C.) under nitrogen. The mixture was stirred at 23° C. for 6h and H₂SO₄ (25.0 mL) was added at 23° C. The mixture was extracted with Et₂O (4×70.0 mL) and the combined organic phases were washed with brine (2×50.0 mL), dried (Na₂SO₄), filtered and concentrated. H₂SO₄ (10.0 mL) was added to the mixture of the residue in MeOH (80.0 mL) at 23° C. under nitrogen. The mixture was refluxed for 16 h, cooled to 23° C. and concentrated. The residue was diluted with Et₂O (100 mL) and water (100 mL). The aq. phase was extracted with Et₂O (4×50.0 mL). The combined organic layers were washed with water (60.0 mL), NaHCO₃ (2×60.0 mL), brine (2×50.0 mL), dried (Na₂SO₄), filtered, and concentrated to provide the title compound as an oil (19 g, 39%). ¹H NMR (400 MHz, CDCl₃) δ 4.34 (dd, J=8.5, 5.8 Hz, 1H), 3.75 (s, 3H), 3.65 (s, 3H), 2.52-2.45 (m, 2H), 2.40-2.30 (m, 1H), 2.26 (m, 1H).

### Step 4: 5-bromo-N-methyl-2-nitro-aniline

Methylamine (56.6 mL, 455 mmol, 33% wt in EtOH) was added to a mixture of 4-bromo-2-fluoro-1-nitro-benzene (50.0 g, 227 mmol) in EtOH (455 mL) at 23° C. under nitrogen. The mixture was stirred at 23° C. for 30 min, filtered and washed with cold EtOH (200 mL) to provide the title compound as a solid (48.2 g, 92%). m/z (ES⁺) [M+H]⁺=231.0, LCMS (A05); t_{R}=2.51 min.

¹H NMR (400 MHz, DMSO-d₆) δ 8.23 (d, J=4.3 Hz, 1H), 7.98 (d, J=9.1 Hz, 1H), 7.17 (d, J=2.0 Hz, 1H), 6.82 (dd, J=9.1, 2.1 Hz, 1H), 2.95 (d, J=5.0 Hz, 3H)

### Step 5: [(3R)-1-[3-(methylamino)-4-nitro-phenyl]pyrrolidin-3-yl]methanol

RuPhos-Pd-G₃ (2.71 g, 3.25 mmol) was added to a mixture of 5-bromo-N-methyl-2-nitro-aniline, (25 g, 108 mmol), tert-butyl-diphenyl-[[(3R)-pyrrolidin-3-yl]methoxy]silane 2,2,2-trifluoroacetic acid_(60.0 g, 119 mmol, 90% purity) and Cs₂CO₃ (106 g, 325 mmol) in PhMe (600 mL) at 23° C. under nitrogen. The mixture was degassed by bubbling nitrogen for 15 min at 23° C., stirred at 100° C. for 19 h, cooled to 23° C., filtered, and concentrated. The product was purified by silica gel chromatography (2×330 g in series cartridges) with hexanes and EtOAc (0-50%) to provide the title compound as a solid (41.0 g, 77%). m/z: ES⁺[M+H]⁺=490.4.

¹H NMR (400 MHz, DMSO-d₆) δ 8.36 (d, J=4.9 Hz, 1H), 7.91 (d, J 9.6 Hz, 1H), 7.64-7.57 (m, 4H), 7.50-7.37 (m, 6H), 6.07 (dd, J 9.6, 2.5 Hz, 1H), 5.50 (d, J=2.4 Hz, 1H), 3.68 (d, J=6.6 Hz, 2H), 3.54-3.46 (m, 1H), 3.46-3.37 (m, 2H), 3.27-3.21 (m, 1H), 2.90 (d, J=5.0 Hz, 3H), 2.64-2.55 (m, 1H), 2.16-2.04 (m, 1H), 1.90-1.79 (m, 1H), 1.01 (s, 9H).

### Step 6: 4-[(3R)-3-ethylpyrrolidin-1-yl]-N₂-methyl-benzene-1,2-diamine

A solution of [(3R)— 1-[3-(methylamino)-4-nitro-phenyl]pyrrolidin-3-yl]methanol, (20.0 g, 40.8 mmol) in THE (100 mL) and EtOH (100 mL) was added to 10% Pd/C (4.4 g, 4.1 mmol, 50% wet.) at 23° C. under nitrogen. The mixture was refluxed and hydrazine hydrate (16 mL, 163 mmol) was added (over 30 min). The mixture was refluxed for 2 h, cooled to 23° C., filtered (Celite), washed with EtOAc (200 mL) and EtOH (200 mL), and concentrated to provide the title compound as an oil (18.0 g, 96%). m/z ESI⁺[M−Ph−tBu+H]⁺=328.16

¹H NMR (400 MHz, DMSO-d₆) δ 7.65-7.59 (m, 4H), 7.48-7.38 (m, 6H), 6.45-6.40 (m, 1H), 5.71 (d, J=2.5 Hz, 1H), 5.66 (dd, J=8.1, 2.5 Hz, 1H), 4.50 (d, J 4.9 Hz, 1H), 3.65 (d, J 6.8 Hz, 2H), 3.33 (br. s, 2H), 3.21 (dd, J 9.1, 7.6 Hz, 1H), 3.14-3.07 (m, 2H), 2.97 (dd, J=9.2, 5.9 Hz, 1H), 2.68 (d, J=4.2 Hz, 3H), 2.56-2.51 (m, 1H), 2.05-1.95 (m, 1H), 1.76-1.67 (m, 1H), 1.01 (s, 9H).

### Step 7: 5-[(3R)-3-ethylpyrrolidin-1-yl]-3-methyl-1H-benzimidazol-2-one

A mixture of triphosgene (8.09 g, 27.3 mmol) in DCM (30 mL) was added to a mixture of ISN-4-[(3R)-3-ethylpyrrolidin-1-yl]-N₂-methyl-benzene-1,2-diamine, (38.0 g, 82.7 mmol) and DIPEA (115 mL, 661 mmol) in DCM (300 mL) at to 0° C. under nitrogen. The mixture was stirred at 0° C. for 30 min and diluted with water (300 mL). The aq. phase was extracted with DCM (2×100 mL), and the combined organic phases were washed with brine (50.0 mL), dried (MgSO₄), filtered, and concentrated. The product was purified by silica gel chromatography (2×330 g cartridge) with DCM and MeOH (0-10%) to provide the title compound as a solid (21 g, 52%). m/z: ES⁺[M+H]⁺=486.4.

### Step 8: Dimethyl 2-[5-[(3R)-3-ethylpyrrolidin-1-yl]-3-methyl-2-oxo-benzimidazol-1-yl]pentanedioate

Dimethyl 2-bromopentanedioate (10.9 g, 30.9 mmol, 68% purity) was added to a mixture of 5-[(3R)-3-ethylpyrrolidin-1-yl]-3-methyl-1H-benzimidazol-2-one, (10.0 g, 20.6 mmol) and Cs₂CO₃ (20.3 g, 62.3 mmol) in DMF (100 mL) at 23° C. under nitrogen. The mixture was stirred at 100° C. for 18 h, cooled to 23° C. and diluted with EtOAc (200 mL) and water (100 mL). The aq. phase was extracted with EtOAc (2×100 mL), and the combined organic phases were washed with brine (2×50 mL), dried (MgSO₄), filtered, and concentrated. The product was purified by silica gel chromatography (220 g cartridge) with hexanes and EtOAc (0-50%) to provide the title compound as a solid (9.00 g, 68%). m/z: ES⁺[M+H]⁺=644.4, LCMS (A05); t_{R}=2.33 min.

### Step 9: 2-[5-[(3R)-3-ethylpyrrolidin-1-yl]-3-methyl-2-oxo-benzimidazol-1-yl]pentanedioic acid

Aq. NaOH (5 M, 14.0 mL, 70.0 mmol) was added to a mixture of Dimethyl 2-[5 [(3R)-3-ethylpyrrolidin-1-yl]-3-methyl-2-oxo-benzimidazol-1-yl]pentanedioate, (9.00 g, 14.0 mmol) in a mixture of THE and water (200 mL, 1:1 v/v) at 23° C. under nitrogen. The mixture was stirred at 23° C. for 1 h and diluted with EtOAc (100 mL) and aq. HCl (1 M, 80.0 mL). The aq. phase was extracted with EtOAc (3×50.0 mL) and the combined organic phases were washed with brine (2×50.0 mL), dried (Na₂SO₄), filtered, and concentrated to provide the title compound as a solid (8.6 g, quant.). ¹H NMR (500 MHz, DMSO-d₆) δ 7.67-7.58 (m, 4H), 7.51-7.36 (m, 6H), 6.93-6.81 (m, 1H), 6.41-6.30 (m, 1H), 6.27-6.17 (m, 1H), 4.95 (dd, J=10.8, 5.0 Hz, 1H), 3.69 (d, J=6.6 Hz, 2H), 3.38-3.31 (m, 1H), 3.29 (s, 3H), 3.27-3.19 (m, 2H), 3.12-3.03 (m, 1H), 2.65-2.55 (m, 1H), 2.41-2.21 (m, 2H), 2.21-2.02 (m, 3H), 1.86-1.79 (m, 1H), 1.02 (s, 9H).

### Step 10: 3-[5-[(3R)-3-ethylpyrrolidin-1-yl]-3-methyl-2-oxo-benzimidazol-1-yl]piperidine-2,6-dione

HATU (6.792 g, 17.9 mmol) was added to a mixture of 2-[5-[(3R)-3-ethylpyrrolidin-1-yl]-3-methyl-2-oxo-benzimidazol-1-yl]pentanedioic acid, (5.0 g, 8.12 mmol), trifluoroacetamide (1.01 g, 8.93 mmol) and DIPEA (5.66 mL, 32.5 mmol) in DMF (50.0 mL) at 23° C. under nitrogen. The mixture was stirred at 23° C. for 18 h and concentrated. The product was purified by silica gel chromatography (120 g cartridge) with DCM and MeOH (0-5%) to provide the title compound as a solid (3.30 g, 68%). ¹H NMR (500 MHz, DMSO-d₆) δ 11.04 (s, 1H), 7.65-7.60 (m, 4H), 7.50-7.39 (m, 6H), 6.93-6.87 (m, 1H), 6.35 (d, J=2.1 Hz, 1H), 6.20 (dd, J=8.6, 2.2 Hz, 1H), 5.26 (dd, J=12.8, 5.4 Hz, 1H), 3.69 (d, J 6.6 Hz, 2H), 3.29 (s, 3H), 3.26-3.20 (m, 2H), 3.08-3.02 (m, 1H), 2.93-2.85 (m, 1H), 2.70 (s, 2H), 2.67-2.55 (m, 2H), 2.13-2.04 (m, 1H), 1.98-1.95 (m, 1H), 1.86-1.76 (m, 1H), 1.02 (s, 9H).

### Step 11: 3-[5-[(3R)-3-(hydroxymethyl)pyrrolidin-1-yl]-3-methyl-2-oxo-benzimidazol-1-yl]piperidine-2,6-dione

TBAF (8.00 mL, 8.00 mmol, 1M in THF) was added to a mixture of 3-[5-[(3R)-3-ethylpyrrolidin-1-yl]-3-methyl-2-oxo-benzimidazol-1-yl]piperidine-2,6-dione, (3.20 g, 5.36 mmol) in THE (20 mL) at 23° C. under nitrogen. The mixture was stirred at 23° C. for 3h and concentrated. The product was purified by silica gel chromatography (220 g cartridge) with DCM and MeOH (0-12%) to provide the title compound as a solid (1.30 g, 67%). m/z: ES⁺[M]⁺=358.2.

¹H NMR (400 MHz, DMSO-d₆) δ 11.03 (s, 1H), 6.89 (d, J=8.5 Hz, 1H), 6.37 (d, J=2.2 Hz, 1H), 6.21 (dd, J=8.6, 2.2 Hz, 1H), 5.25 (dd, J=12.9, 5.4 Hz, 1H), 4.69 (t, J=5.2 Hz, 1H), 3.48-3.36 (m, 2H), 3.37-3.32 (m, 1H), 3.29 (s, 3H), 3.27-3.15 (m, 2H), 3.05-2.97 (m, 1H), 2.95-2.83 (m, 1H), 2.73-2.55 (m, 2H), 2.48-2.37 (m, 1H), 2.08-1.92 (m, 2H), 1.79-1.68 (m, 1H).

### Step 12: (3R)-1-(1-(2,6-dioxopiperidin-3-yl)-3-methyl-2-oxo-2,3-dihydro-1H-benzo[d]imidazol-5-yl)pyrrolidine-3-carbaldehyde

To a mixture of (3RS)-3{-5-[(3R)-3-(hydroxymethyl)pyrrolidin-1-yl]-3-methyl-2-oxo-1,3-benzodiazol-1-yl}piperidine-2,6-dione (33.50 mg, 0.09 mmol) in DMSO (1.00 mL)was added triethylamine (0.26 mL, 0.19 g, 1.87 mmol) followed by sulfur trioxide pyridine complex (148.77 mg, 0.93 mmol). After 25 minutes, water was added and the mixture was extracted with DCM twice. The combined organic layers were concentrated to give the title compound without further purification. m/z: ES⁺[M]⁺=357.2.

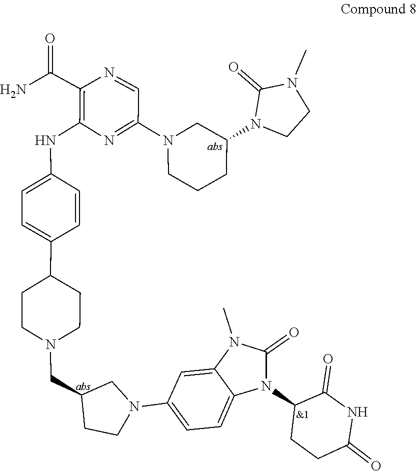 [SMILES: CN1CCN([C@@H]2CCCN(c3cnc(C(N)=O)c(Nc4ccc(C5CCN(C[C@@H]6CCN(c7ccc8c(c7)n(C)c(=O)n8[C@@H]7CCC(=O)NC7=O)C6)CC5)cc4)n3)C2)C1=O]

To a mixture of 5-[(3R)-3-(3-methyl-2-oxoimidazolidin-1-yl)piperidin-1-yl]-3-{[4-(piperidin-4-yl)phenyl]amino}pyrazine-2-carboxamide (27.28 mg, 0.06 mmol) in DCM (1.00 mL) was added N,N-diisopropylethylamine (0.12 mL, 0.68 mmol). Tested basic to pH paper. The resulting suspension was added to (3R)-1-(1-(2,6-dioxopiperidin-3-yl)-3-methyl-2-oxo-2,3-dihydro-1H-benzo[d]imidazol-5-yl)pyrrolidine-3-carbaldehyde (20.31 mg, 0.06 mmol) followed by sodium triacetoxyborohydride (36.24 mg, 0.17 mmol). After 45 minutes, water was added. The mixture was extracted twice with DCM. The combined organic layers were concentrated and purified by HPLC (MeCN/H₂O with 0.1% TFA) to afford 3-((4-(1 (((3 S)-1-(1-(2,6-dioxopiperidin-3-yl)-3-methyl-2-oxo-2,3-dihydro-1H-benzo[d]imidazol-5-yl)pyrrolidin-3-yl)methyl)piperidin-4-yl)phenyl)amino)-5-((R)-3-(3-methyl-2-oxoimidazolidin-1-yl)piperidin-1-yl)pyrazine-2-carboxamide (22.3 mg, 47%). ¹H NMR (500 MHz, Acetonitrile-d3) δ 11.14 (s, 1H), 9.07 (s, 1H), 8.92 (s, 1H), 7.66-7.56 (m, 3H), 7.43 (s, 1H), 7.23 (d, J=8.3 Hz, 2H), 6.85 (d, J=8.4 Hz, 1H), 6.44 (s, 1H), 6.36 (s, 1H), 5.83 (s, 1H), 5.15 (dd, J=12.8, 5.2 Hz, 1H), 4.42 (d, J=12.4 Hz, 1H), 4.31 (d, J=13.5 Hz, 1H), 3.77-3.67 (m, 3H), 3.62-3.56 (m, 1H), 3.50-3.12 (m, 8H), 3.13-2.96 (m, 4H), 2.95-2.68 (m, 6H), 2.69-2.19 (m, 6H), 2.18-2.09 (m, 5H), 1.97-1.85 (m, 5H), 1.70-1.64 (m, 1H). LCMS: C₄₃H₅₄N₁₂O₅ requires 818, found: m/z=819 [M+H]⁺.

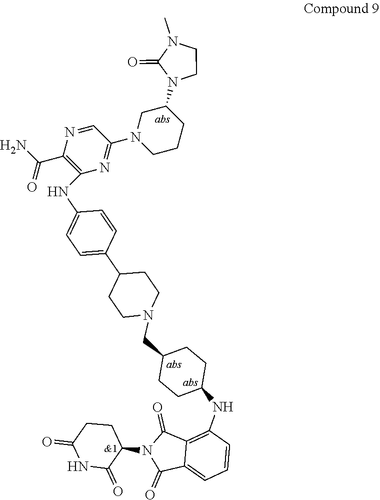 [SMILES: CN1CCN([C@@H]2CCCN(c3cnc(C(N)=O)c(Nc4ccc(C5CCN(C[C@H]6CC[C@@H](Nc7cccc8c7C(=O)N([C@@H]7CCC(=O)NC7=O)C8=O)CC6)CC5)cc4)n3)C2)C1=O]

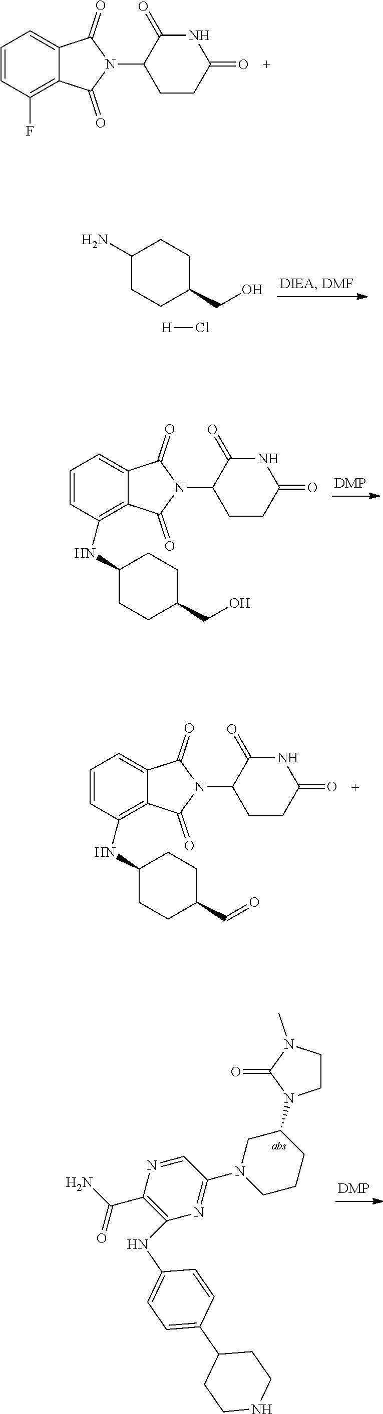

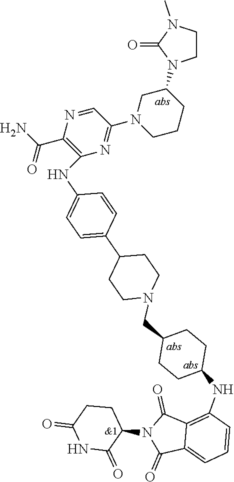 [SMILES: CN1CCN([C@@H]2CCCN(c3cnc(C(N)=O)c(Nc4ccc(C5CCN(C[C@H]6CC[C@@H](Nc7cccc8c7C(=O)N([C@@H]7CCC(=O)NC7=O)C8=O)CC6)CC5)cc4)n3)C2)C1=O]

A mixture of [(1s,4s)-4-aminocyclohexyl]methanol hydrochloride (230 mg, 1.39 mmol), 2-(2,6-dioxopiperidin-3-yl)-4-fluoroisoindole-1,3-dione (319 mg, 1.15 mmol), N,N-diisopropylethylamine (0.60 mL, 3.46 mmol) and DMF (10 mL) was allowed to stir at 80° C. for 7 h. EtOAc and H₂O were added. The organic layer was dried with MgSO₄, filtered, concentrated and purified by MPLC (0-10% MeOH in CH₂Cl₂) to afford 2-(2,6-dioxopiperidin-3-yl)-4-{[(1 s,4s)-4-(hydroxymethyl)cyclohexyl]amino}isoindole-1,3-dione (0.135 g, 30.3%). LCMS: C₂₀H₂₃N₃O₅ requires 385, found: m/z=386 [M+H]⁺.

A mixture of 1,1-bis(acetyloxy)-3-oxo-1lambda5,2-benziodaoxol-1-yl acetate (297 mg, 0.70 mmol), 2-(2,6-dioxopiperidin-3-yl)-4-{[(1 s,4s)-4-(hydroxymethyl)cyclohexyl]amino}isoindole-1,3-dione (135 mg, 0.35 mmol) and CH₂Cl₁₋₂ (12 mL) was allowed to stir at room temperature for 1 h. The mixture was concentrated and purified by MPLC (0-10% MeOH in CH₂Cl₂) to afford (1s,4s)-4-{[2-(2,6-dioxopiperidin-3-yl)-1,3-dioxoisoindol-4-yl]amino}cyclohexane-1-carbaldehyde (0.1320 g, 98.3%). LCMS: C₂₀H₂₁N₃O₅ requires 383, found: m/z=384 [M+H]⁺.

Sodium bis(acetyloxy)boranuidyl acetate (22 mg, 0.10 mmol) was added to a mixture of 5-[(3R)-3-(3-methyl-2-oxoimidazolidin-1-yl)piperidin-1-yl]-3-{[4-(piperidin-4-yl)phenyl]amino}pyrazine-2-carboxamide (25 mg, 0.05 mmol), triethylamine (0.06 mL, 0.42 mmol), (1 s,4s)-4-{[2-(2,6-dioxopiperidin-3-yl)-1,3-dioxoisoindol-4-yl]amino}cyclohexane-1-carbaldehyde (30 mg, 0.08 mmol) and DCE (1 mL). The mixture was allowed to stir at room temperature for 1 h. The mixture was filtered washing with CH₂Cl₂/EtOAc, concentrated and purified by HPLC (5-95% MeCN in H₂O with 0.1% TFA) to afford 5-[(3R)-3-(3-methyl-2-oxoimidazolidin-1-yl)piperidin-1-yl]-3-{[4-(1-{[(1 s,4s)-4-{[2-(2,6-dioxopiperidin-3-yl)-1,3-dioxoisoindol-4-yl]amino}cyclohexyl]methyl}piperidin-4-yl)phenyl]amino}pyrazine-2-carboxamide (0.0082 g, 18.6%). ¹H NMR (500 MHz, Acetonitrile-d3) δ 11.13 (s, 1H), 8.93 (s, 1H), 8.68 (s, 1H), 7.68-7.53 (m, 4H), 7.43 (s, 1H), 7.22 (d, J=8.1 Hz, 2H), 7.08 (d, J=7.9 Hz, 2H), 6.47 (s, 1H), 5.82 (s, 1H), 4.98 (dd, J=12.4, 5.4 Hz, 1H), 4.42 (d, J=13.2 Hz, 1H), 4.32 (d, J=13.5 Hz, 1H), 3.86 (s, 1H), 3.69 (d, J=12.9 Hz, 3H), 3.48-3.25 (m, 4H), 3.15-2.94 (m, 6H), 2.89-2.64 (m, 7H), 2.09 (s, 5H), 1.93-1.39 (m, 15H). LCMS: C₄₅H₅₅N₁₁O₆ requires 845, found: m/z=846 [M+H]⁺.

### Biological Example 1

Compounds 1-24 were tested for BTK degradation in TMD8 cells using the protocol described in PCT publication WO2020/081450 (Example 66). All degraded BTK with DC₅₀ of less than 100 nM. A subset of these compounds were tested for BTK degradation in other cells and were shown to degrade BTK in a dose dependent manner in microglia, monocytes and macrophases.

In addition, Compounds 1-24 were assayed for IMiD activity. Frozen human peripheral blood mononuclear cells (PBMCs) were thawed and treated with DMSO or compound for 24 hours and then fixed and permeabilized using a Foxp3/Transcription Factor Fixation/Permeabilization Kit (eBioscience, 00-5523). Cells were stained with fluorophore-conjugated antibodies against CD20 (Biolegend 302330), CD3 (BD Pharmingen 552127), and Aiolos (Biolegend 371106). An additional set of DMSO-treated PBMCs was stained for CD20, CD3, and an AlexaFluor 647-conjugated mouse IgG1 isotype control antibody (Biolegend 400136). Stained cells were run on an Attune NxT Acoustic Focusing Flow Cytometer (Thermo-Fisher A29004), and data was analyzed using FlowJo (v10.5.3) and GraphPad Prism (v7.00) software. Single lymphocytes were gated for B cells (CD20+CD3-) and T cells (CD3+CD20-), and the geometric mean fluorescence intensity (MFI) of Aiolos was calculated for each population. The MFI of the isotype control was calculated for each population and used to quantify background staining. Percent Aiolos degradation was calculated for each compound-treated sample using the following equation:

$$\% Degradation = 100 * ( Sample M F I - Isotype M F I ) / ( DMSO M F I - Isotype M F I )$$

Each compound showed little or no measurable IMiD activity, as shown in the table below. DC50 is the compound concentration degrading 50% of Aiolos. Dmax is the maximum percent Aiolos degradation in the assay. Compounds 20-22 showed little or no measurable IMiD activity by western blotting (data not shown).

| Compound | IMiD activity DC50/μM | Dmax/% |
|---|---|---|
| 1 | 0.631 | 18.7 |
| 2 | 0.596 | 17.9 |
| 3 | 2.00 | 3.2 |
| 4 | 0.344 | 28.3 |
| 5 | 0.468 | 10.1 |
| 6 | 2.00 | 1.1 |
| 7 | 2.00 | 4.6 |
| 8 | 2.00 | 1.0 |
| 9 | 0.0966 | 30.8 |
| 10 | 2.00 | 3.2 |
| 11 | 2.00 | 1.9 |
| 12 | 2.00 | 5.3 |
| 13 | 0.00289 | 4.9 |
| 14 | 0.0376 | 30.4 |
| 15 | 0.345 | 27.4 |
| 16 | 2.00 | 23.6 |
| 17 | 2.00 | 15.1 |
| 18 | 2.65 | 28.2 |
| 19 | 2.00 | 9.1 |

### Biological Example 2

Compound 1 provided significant benefit in the mouse CIA model with minimal effect on body weight.

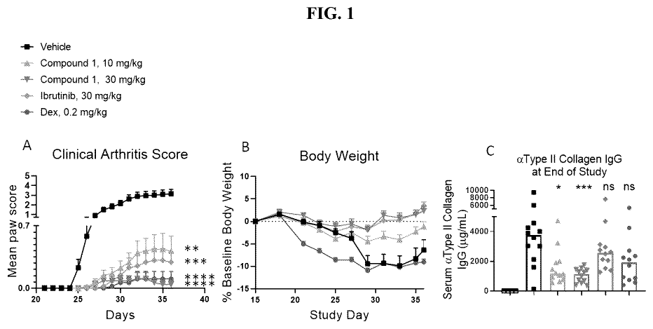

Immunization with Collagen Type II (CII) in an adjuvant induces polyarthritis in rodents. This model known as the collagen-induced arthritis (CIA) model, is widely used for studying arthritis and resemble many aspect of the human disease. High level of anti-collagen II correlates with disease severity. In FIG. 1 CIA was induced by immunization with type II collagen in complete Freund's adjuvant on Day 0. A second boost was given on Day 21. Treatment with vehicle or therapeutic agents was initiated on Day 18 and continued to day 35. At the end of the study on Day 36, serum was collected.

A) Daily oral treatment with Compound 1 at 30 mg/kg resulted in lower mean arthritis score than ibrutinib at 30 mg/kg. Compound leffect provided similar clinical benefit as dexamethasone with minimal body weight loss (B) as compared to dexamethasone and vehicle. Significance of clinical arthritis score (A) was determined from the area under the curve (AUC) of mean paw scores calculated for individual mice. (C) Serum levels of anti-type II collagen IgG. Statistical significance was determined between vehicle control and treated groups with one-way Kruskal-Wallis ANOVA and Dunn's multiple comparisons test.

Compound 1 also resulted in lower mean arthritis score than standard of care agents: rilzabrutinib 10 mg/kg and 30 mg/kg; tofacitinib 30 mg/kg BID and Enbrel 10 mg/kg in the same model.

### Biological Example 3

Compound 1 shows dose-dependent reduction of clinical scores in EAE that is superior to Ibrutinib.

Experimental autoimmune encephalomyelitis (EAE) is an inflammatory demyelinating disease of the central nervous system (CNS).

EAE was induced in C57BL/6 mice by immunization with an emulsion of MOG1-125 in complete Freund's adjuvant (CFA), followed by administration of pertussis toxin in PBS, first on the day of immunization (Day 0) and then again the following day (Day 1).

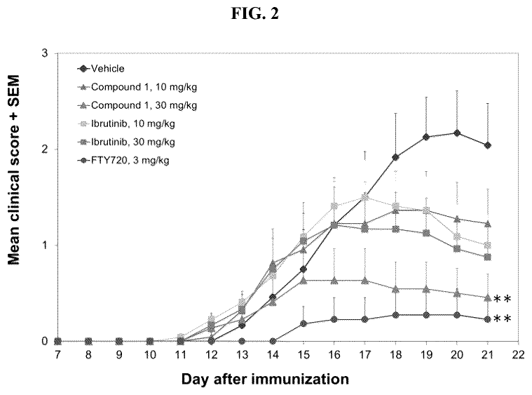

Dosing of all mice started on Day −1 and continued through Day 22. Fingolimod (FTY720, Gilenya) is the a commonly used positive control in this model. In FIG. 2, prophylactic treatment with daily oral Compound 1 showed dose-dependent reduction of EAE clinical scores.

Compound 1 treatment at 30 mg/kg resulted in a lower mean clinical score than ibrutinib dosed at 30 mg/kg. Statistical significance was determined between vehicle control and treated groups based on the end clinical scores using Wilcoxon's non-parametric test.

Histological analysis was performed on spinal cords collected on day 22 from mice treated with either Compound 1 at 30 mg/kg or ibrutinib at 30 mg/kg.

Compound 1 dosed at 30 mg/kg resulted in significant reduction in the number of inflammatory foci or apoptotic cells per section of spinal cord and a significantly reduced demyelination score compared to vehicle control.

Treatment with ibrutinib at 30 mg/kg did not result in statistically significant changes in histological characteristics compared to vehicle control.

Statistical analysis: Demyelination (anti-MBP) with Wilcoxon's non-parametric test; inflammatory foci or apoptosis with two-tailed Student's t-test.

| | Inflammatory foci/ | | Demyelination | | Apoptotic cells/ | |
|---|---|---|---|---|---|---|
| Treatment | section +/− SD | p value | score +/− SD | p value | section +/− SD | p value |
| Vehicle | 2.7 +/− 2.6 | | 0.8 +/− 0.7 | | 0.5 +/− 0.6 | |
| Compound 1 | 0.2 +/− 0.7 | 0.0067 | 0.1 +/− 0.1 | 0.0079 | 0.1 +/− 0.2 | 0.0227 |
| 30 mg/kg | | | | | | |
| Ibrutinib | 1.0 +/− 1.4 | 0.0640 | 0.4 +/− 0.6 | 0.2089 | 0.4 +/− 0.7 | 0.6777 |
| 30 mg/kg | | | | | | |

### Biological Example 4

MRL/lpr mice, which are characterized genetically by defective Fas-mediated apoptosis, exhibit spontaneous systemic autoimmune disease that mimics human SLE, including predominance in female animals, circulating nuclear auto-antibodies, and pathology in multiple end organs. Compound 1 significantly ameliorated kidney disease in the MRL/lpr model.

Treatment of MRL-lpr mice with daily oral administered Compound 1 starting at 10 weeks of age resulted in dose-dependent reduction of urine protein score. Compound 1 but not Ibrutinib given at 30 mg/kg results in a statistically significant reduction of protein urine score. Dexamethasone was used as positive control. Statistical significance is shown as an asterisk next to the group symbol in the figure legend of treatments with P<0.05 as determined between vehicle control and treated groups based on the Urine Protein Score AUC using a Kruskal-Wallis test with Dunn's post-hoc analysis.

Compound 1 at 30 mg/kg and Ibrutinib at 10 mg/kg result in a statistically significant reduction in anti-ds DNA autoantibody measured in end of study serum. Statistical significance was determined between vehicle control and treated groups by ANOVA with Dunnett's post-hoc analysis.

Histological examination of H&E-stained kidney tissue from treated mice harvest at the end of the study, revealed Compound 1 is more effective than Ibrutinib in decreasing the size of the glomeruli and the overall summed histology score in treated mice. Glomeruli size is a quantitative measure that correlates well with the histopathological grade and severity of nephritis in this model. Statistical significance was determined between vehicle control and treated groups by ANOVA with Dunnett's post-hoc analysis (glomerulus diameter) or with a Kruskal-Wallis test with Dunn's post-hoc analysis (summed scores). * p<0.05, ** p<0.01, *** p<0.001**** p<0.0001.

### Biological Example 5

Evaluation of Compound 1, Comparator Compound and Ibrutinib dosing effect on B cells compartment. Comparator Compound has IMiD activity and was prepared as described in PCT/US2019/56112, filed Oct. 14, 2019.

Female C57BL/6 mice were dosed with Compound 1, Comparator Compound or Ibrutinib formulated in water bottle at 30 mg/kg. A separate group of mice received vehicle formulation as control. Blood spleens and femurs for each treated mice were harvested at the end of the study. Peritoneal lavage was also performed with PBS to harvest peritoneal resident lymphocyte.

Demonstrate that both Compound 1 and Comparator Compound treatment resulted in a robust BTK degradation in blood, spleens, peritoneum and bone marrow of treated mice. Ibrutinib induce a minimal reduction of BTK level in B cells. To evaluate level of BTK in B cells, Single cell suspensions from each tissue were permeabilized and stained for lineage markers and intracellular BTK. B cells were gated on B220+ TCRbeta−, and T cells were gated on B220− and TCRbeta+. Percent BTK remaining was calculated by subtracting the mean fluorescence intensity (MFI) of BTK in T cells, which do not express BTK, from the mean fluorescence intensity of BTK in B cells, and then normalizing this value to the vehicle group.

Treatment with Compound 1, Comparator Compound or Ibrutinib does not significantly affect B cells percentage in blood, spleens, peritoneum and bone marrow of treated animals.

The results show that BTK inhibitor, Ibrutinib or BTK degrader, Comparator Compound do not alter plasma cells number in bone marrow of treated animals. However, treatment with Compound 1 significantly reduce plasma cells in bone marrow of treated animals. It is plausible that Compound 1 affects one or multiple proteins that are involved either in generation or the survival of plasma cells and that this effect is independent from its ability to degrade the BTK protein. Left, representative dot plots showing the gating strategy to identify plasma cells in bone marrow. Right histograms showing cumulative data from multiple treated animals.

An ordinary one-way ANOVA with Tukey's multiple comparisons test was performed, and all groups were compared to each other. * p<0.05, ** p<0.01,**** p<0.001. All comparisons without an asterisk were not significant.

### Biological Example 6

The impact of Compound 1 and Comparator Compound and Ibrutinib in Ab affinity maturation and plasma cell generation was assessed upon immunization with the T cell-dependent model Ag NP-KLH. The results show that BTK inhibitor Ibrutinib or BTK degrader Comparator Compound have no effect on the generation plasma cells while Compound 1 significantly decrease the number of plasma cells generated upon immunization.

In T cell dependent immunization models, IgG1+ antibody responses are not significantly reduced by BTK inhibitors such as ibrutinib. (www.jimmunol.org/content/193/1/185). It is therefore surprising to find what seems a specific effect of Compound 1. It is plausible that Compound 1 affects one or multiple proteins that are involved either in generation or plasma cells survival and that this effect is independent from its ability to degrade the BTK protein. The success of current B cell targeting therapies emphasizes the important roles B cells play in the pathogenesis of autoimmune diseases. Hence, therapies that specifically deplete pathogenic B cells such as plasma cells, could hold great potential for the treatment of autoimmune diseases. A treatment able to affect BTK level and also deplete plasma cells while sparing other B cell populations is predicted to have a therapeutic effect on autoantibody mediated disease.

Mice were immunized by intraperitoneal injection of 200 μL of immunogen containing 100 μg NP-32-KLH (Biosearch Technologies, Cat. N-5060-25,Lot. 156285-01) and 1% Alum (Alhydrogel, 2%, Invivogen Cat. Vac-alu-250, Batch 0001715532, Exp. July 2022), prepared in phosphate buffered saline. Group 1 mice were not immunized and used as control. Mice in Group 2 to 5 were dosed orally once a day from Day 1 to Day 7 with compounds or vehicle for six additional days and then sacrificed the following day. Splenocytes and bone marrow were harvested 24 hours following the final dosing of compound on Day 7. Spleens harvested at the end of the study were dissociated, and red blood cells were lysed with ammonium-chloride-potassium (ACK) lysis buffer. 1 million cells per tissue were stained for each flow cytometry panel.

Compound 1 and Comparator Compound treatment resulted in a robust BTK degradation in spleens of treated animals. Cells were permeabilized and stained for lineage markers and intracellular BTK. B cells were gated on CD19+ TCRbeta−, and T cells were gated on CD19− and TCRbeta+. Percent BTK remaining was calculated by subtracting the mean fluorescence intensity (MFI) of BTK in T cells, which do not express BTK, from the mean fluorescence intensity of BTK in B cells, and then normalizing this value to the naive group. 24 hours after the final dose of compound, BTK levels were suppressed to 13.4% of baseline in the Compound 1 group and 21.1% of baseline in the Comparator Compound group.

Ibrutinib mediated BTK inhibition or Compound 1 or Comparator Compound mediated BTK degradation do not affect number of B cells in spleen or germinal center formation following immunization. Splenocytes were stained for lineage markers with fluorophore-conjugated antibodies and for NP hapten reactivity with NP-conjugated to phycoerythrin (NP-PE). Live single lymphocytes were gated for B cells (CD19+B220+) and then gated for IgD+ and IgD− B cells. IgD− B cells were then gated for germinal center B cells (Fas+GL-7+), and germinal center B cells were gated for NP+ cells on the basis of staining with NP-PE.

Compound 1 treatment significantly decrease number of total Plasma cells and IgG1+ plasma cells in spleen of immunized mice.

An ordinary one-way ANOVA with Tukey's multiple comparisons test was performed, and all groups were compared to each other. * p<0.05, ** p<0.01,**** p<0.001. All comparisons without an asterisk were not significant.

### Biological Example 7

The effect of compound 1 was measured in a mouse model of well-established arthritis. Compound 1 given at 10 mg/kg or 30 mg/kg strongly suppressed disease when started after arthritis was already established. Compound 1 was able to control disease to a similar extent as ibrutinib (both given at 30 mg/kg dose) but more potently than anti-TNF-alpha blocking antibody (Enbrel) or BTK inhibitors rilzabrutinib and tofacitinib.

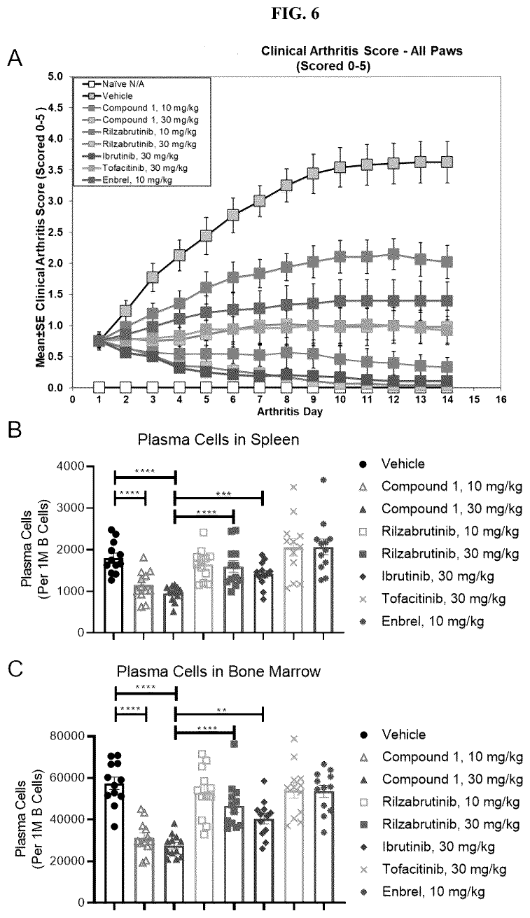

(A) Arthritis was induced in male DBA/1 mice by two intradermal injections of type II collagen in Freund's complete adjuvant on study days 0 and 21, then mice were randomized and enrolled into treatment arms on study days 25-27 as the first clinical signs of arthritis occurred and treatment was initiated (defined as Arthritis Day 1). In FIG. 6A, once daily oral treatment was performed for vehicle, compound 1, rilzabrutinib, or ibrutinib at the indicated doses; Tofacitinib was administered orally twice daily at 30 mg/kg for each dose; Enbrel was administered by daily intraperitoneal injection at 10 mg/kg. Treatment continued until Arthritis Day 14 and the study ended on Arthritis Day 15.

(B) Spleens harvested at the end of the study were dissociated, and red blood cells were lysed with ammonium-chloride-potassium (ACK) lysis buffer. 1.5 million cells per spleen were stained for flow cytometry, and live single lymphocytes were gated for plasma cells (CD138+ IgD−). Plasma cells numbers were normalized to total B cells in spleen (CD19+B220+). As shown in FIG. 6B, mice treated with compound 1 at both doses had significantly fewer plasma cells than vehicle-treated mice, and mice treated with 30 mg/kg compound 1 had significantly fewer plasma cells than mice treated with an equivalent dose of BTK inhibitors (rilzabrutinib and ibrutinib).

(C) Bone marrow was isolated from femurs harvested at the end of the study, and samples were processed and analyzed as in (B). As shown in FIG. 6C, compound 1 significantly reduced plasma cell numbers in the bone marrow compared to vehicle, as well as compared to an equivalent doses of BTK inhibitors (rilzabrutinib and ibrutinib).

In (B) and (C), ordinary one-way ANOVAs with Dunnett's multiple comparisons tests were performed. Vehicle was compared to both doses of Compound 1, and 30 mg/kg Compound 1 was compared to 30 mg/kg rilzabrutinib and 30 mg/kg ibrutinib. ** p<0.01, *** p<0.001, ****p<0.001.

### Biological Example 8

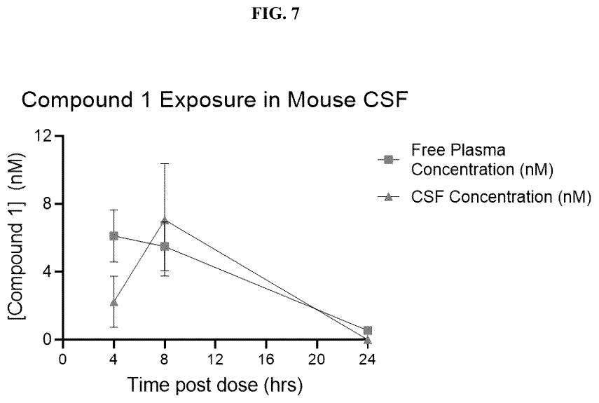

Certain bifunctional BRK degrader compounds cross the blood brain barrier. The ability of compound 1 to cross the blood brain barrier was evaluated in a mouse model. As shown in FIG. 7, compound 1 crosses the blood brain barrier and demonstrates exposure in cerebral spinal fluid (CSF) following administration of a single oral dose in mice.

Male C57BL/6 mice were administered Compound 1 orally at 300 mg/kg and 4, 8, or 24 hours after dosing CSF and plasma was collected from three mice per time point. The concentration of Compound 1 in CSF and plasma was determined by LC/MS/MS; free plasma concentration was calculated by multiplying total plasma concentration by the previously determined percentage of Compound 1 unbound to plasma protein.

### Biological Example 9

The effect of compound 1 on brain tumors was evaluated in a cell line model. Compound 1 treatment in mice bearing intracranial TMD8 tumors results in degradation of tumor-expressed BTK and reduced tumor burden.

TMD8 is a human activated B cell (ABC)-type diffuse large B cell lymphoma (DLBCL) cell line that is dependent on BTK signaling for survival (Davis, R. E., Ngo, V. N., Lenz, G., Tolar, P., Young, R. M., Romesser, P. B., Staudt, L. M. (2010). Chronic active B-cell-receptor signalling in diffuse large B-cell lymphoma. Nature, 463(7277), 88-92. doi: 10.1038/nature08638).

TMD8 cells were implanted into female CB.17 SCID mice by intracranial injection of 5E5 TMD8 cells. Treatment with compound 1 begun the following day. Mice were treated orally every day for twelve days with either vehicle or 90 mg/kg of Compound 1. Brains were then harvested, dissociated into single cell suspensions and BTK level in TMD8 cells was assessed by flow cytometry. TMD8 cells in dissociated brain tissue were identified by human-specific surface markers (CD20+HLA+) and total numbers of TMD8 cells per brain is reported in (A, tumor burden). Percent BTK remaining in TMD8 cells (B) was calculated according to the below formula:

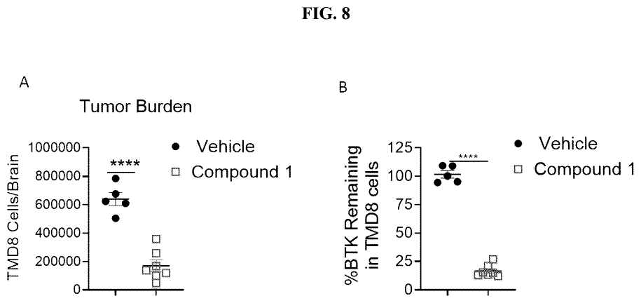

100*(tumor BTK MFI−background MFI)/(peak MFI−background MFI),

where background MFI and peak MFI were calculated using TMD8 cells treated, in vitro, overnight with 1 μM compound 1 or DMSO control, respectively. In FIG. 8,**** p<0.0001; Welch's t-test was used to compare mice treated with compound 1 to mice treated with vehicle. As shown in FIG. 8, compound 1 significantly reduced TMD8 tumor cells (FIG. 8A) and degraded BTK (FIG. 8B) in vitro.

### OTHER EMBODIMENTS

It is to be understood that the foregoing description is intended to illustrate and not limit the scope of this disclosure, which is defined by the scope of the appended claims. Other aspects, advantages, and modifications are within the scope of the following claims.

What is claimed is:

1. A method of treating or preventing a disease in a subject in need thereof, comprising the step of administering to the subject an amount of a bifunctional compound, wherein said bifunctional compound is capable of inducing proteolytic degradation of Bruton's tyrosine kinase, wherein said compound has little or no IMiD activity, and wherein said amount is effective to treat or prevent the disease.

2. The method of claim 1 wherein said administering is chronically administering.

3. The method of any of the previous claims, wherein the compound is administered for two or more weeks of treatment.

4. The method of any of the previous claims, wherein the compound is administered for one or more months.

5. The method of any of the previous claims, wherein the compound is administered for one or more years.

6. The method of any of the previous claims, wherein the compound is administered at 100-600 mg/kg body weight/day.

7. The method of any of the previous claims, wherein the compound is administered at 200-600 mg/kg body weight/day.

8. The method of any of the previous claims, wherein the compound is administered at 300-600 mg/kg body weight/day.

9. The method of any of the previous claims, wherein the frequency of chronically administrating is daily.

10. The method of any of the previous claims, wherein the frequency of chronically administering is twice a day.

11. The method of any of the previous claims, wherein the frequency of chronically administering is thrice a day.

12. The method of any of the previous claims, wherein the frequency of chronically administering is frice a day.

13. The method of any of the previous claims, wherein the frequency of chronically administering is once a week.

14. The method of any of the previous claims, wherein the frequency of chronically administering is twice a week.

15. The method of any of the previous claims, wherein the disease is cancer, an autoimmune disease, or an inflammatory disease.

16. The method of any of the previous claims, wherein the cancer comprises a solid tumor.

17. The method of any of the previous claims, wherein the cancer is a B cell malignancy.

18. The method of any of the previous claims, wherein the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), central nervous system (CNS) lymphoma, metastatic melanoma, squamous cell carcinoma of the head and neck (HNSCC), non-small cell lung cancer (NSCLC), platinum-resistant epithelial ovarian cancer (EOC), gastric cancer, metastatic castrate-resistant prostate cancer (mCRPC), triple-negative breast cancer (TNBC), muscle-invasive urothelial cancer, mesothelioma, cervical cancer, microsatellite stable colorectal cancer (MSS CRC), and multiple myeloma (MM).

19. The method of any of the previous claims wherein the disease is brain cancer.

20. The method of any of the previous claims wherein the disease is selected from the group consisting of acoustic neuroma, astrocytoma, pilocytic astrocytoma, juvenile pilocytic astrocytoma, low-grade astrocytoma, anaplastic astrocytoma, glioblastoma, chordoma, CNS lymphoma, craniopharyngioma, glioma, brain stem glioma, ependymoma, mixed glioma, optic nerve glioma, subependymoma, medulloblastoma, meningioma, metastatic brain tumors, oligodendroglioma, pituitary tumor, primitive neuroectodermal (PNET), rhabdoid tumor, and schwannoma.

21. The method of any of the previous claims wherein the disease is selected from the group consisting of Waldenstrom's macroglobulinemia, marginal zone lymphoma, mantle cell lymphoma, primary central nervous system lymphoma, and chronic lymphocytic leukemia.

22. The method of any of the previous claims, wherein the autoimmune disease is selected from the group consisting of graft-versus-host disease (GVHD), acute graft-versus-host disease, and immune thrombocytopenic purpura (ITP).

23. The method of any of the previous claims, wherein the autoimmune disease is selected from the group consisting of warm autoimmune hemolytic anemia (wAIHA), systemic sclerosis, and systemic sclerosis membranous nephropathy.

24. The method of any of the previous claims, wherein the subject has a C481 mutant Bruton's tyrosine kinase.

25. The method of any of the previous claims, wherein the cancer is ibrutinib-resistant.

26. The method of any of the previous claims, wherein the bifunctional compound is a compound of Formula (A) 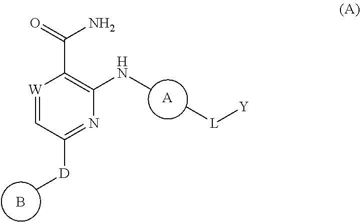
or a pharmaceutically acceptable salt thereof, wherein
W is CH or N;
D is a bond or —NH—;
Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S, wherein Ring A is optionally and independently substituted with up to three substituents selected from halo, —CN, —COOH, NH₂, and optionally substituted C₁₋₆ alkyl;
Ring B is a phenyl, a 5-6 membered heteroaryl, a 4-6 membered heterocycloalkyl, or a 8-10 membered spiro bicyclic heterocycle, wherein Ring B is optionally substituted, and wherein the heteroaryl and heterocycloalkyl of Ring B has one to three heteroatoms independently selected from N, O, or S;
L is —X¹—X²—X³—X⁴—X⁵—;
X¹ is a bond, —C(O)—N(R)—, —N(R)—C(O)—, —(O—CH₂—CH₂)ₘ—, —O(C6H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃;
X² is a bond, —(O—CH₂—CH₂)ₙ (CH₂—CH₂—O)ₙ, N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S;
X⁵ is a bond, —C₁₋₄ alkyl-, —N(R)—, —O—, —C(O)—, or —C(O)—N(R)—;
each R is independently hydrogen or —C₁₋₃ alkyl; and
each of m, n, and p is independently an integer from one to three; and
Y is 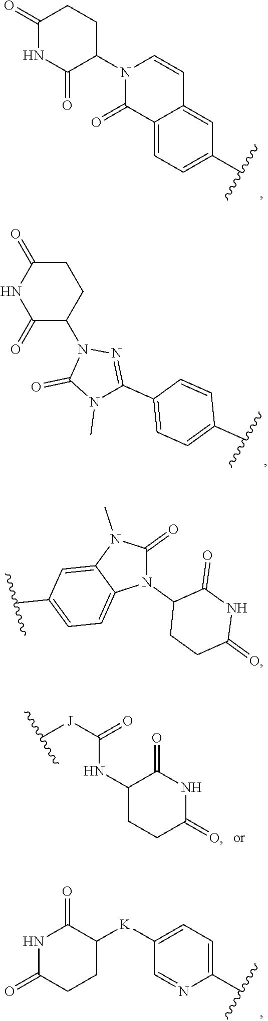
wherein
each J is independently aryl or heteroaryl; and
each K is independently absent, —CH₂—, —NH—, —NMe—, or —O—.

27. The method of claim 26, wherein Ring B is an optionally substituted 5-6 membered heterocycloalkyl having one to two nitrogen atoms.

28. The method of claim 26, wherein Ring B is an optionally substituted 5-6 membered heteroaryl having one to two heteroatoms independently selected from N and S.

29. The method of claim 26, wherein Ring B is 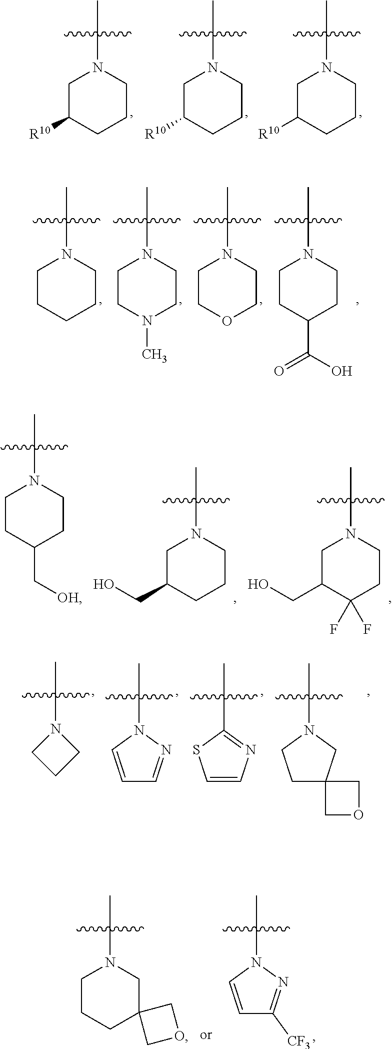
wherein R¹⁰ is 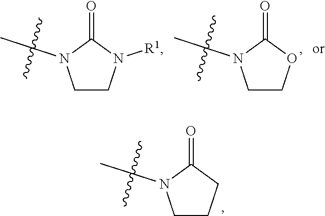
and wherein R¹ is a C₁₋₄ alkyl group.

30. The method of claim 26 or 29, wherein Ring B is 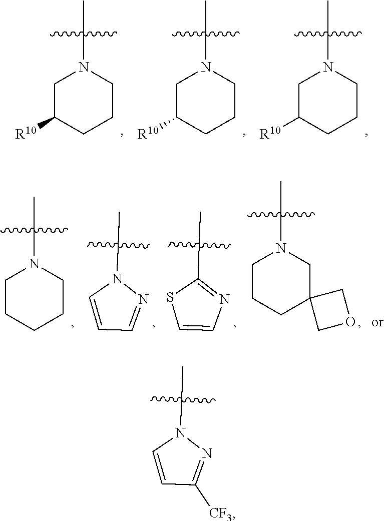
wherein R¹⁰ is

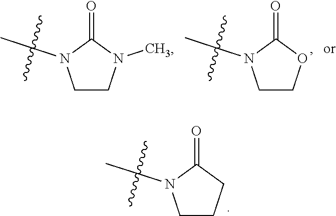 [SMILES: *N1CCN(C)C1=O ; *N1CCOC1=O ; *N1CCCC1=O]

31. The method of claim 29 or 30, wherein Ring B is

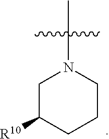

32. The method of any one of claims 29-31, wherein R¹⁰ is

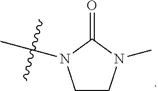 [SMILES: *N1CCN(C)C1=O]

33. The method of any one of claims 26-32, wherein Ring A is 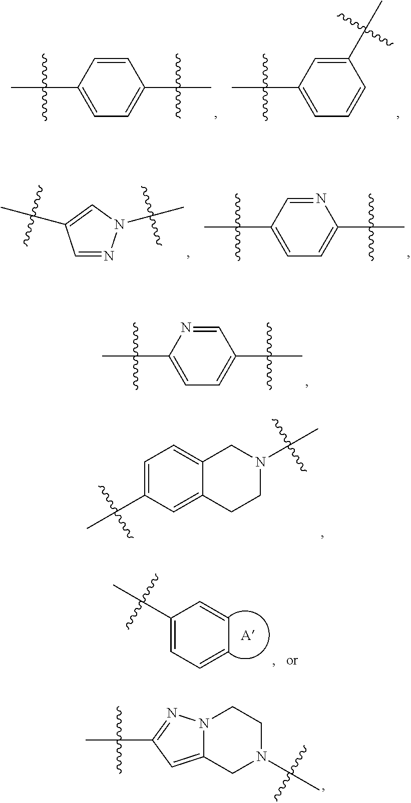
wherein Ring A′ together with the phenyl ring to which Ring A′ is fused forms a 9-10 membered bicyclic aryl or a 9-10 membered bicyclic heteroaryl wherein the bicyclic heteroaryl has one to three heteroatoms independently selected from N, O, or S.

34. The method of any one of claims 26-33, wherein Ring A is

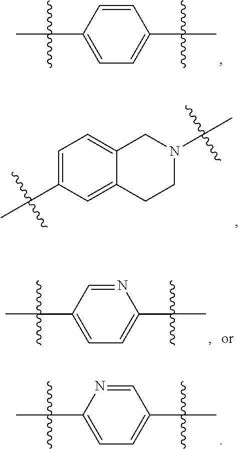

35. The method of any one of claims 26-34, or a pharmaceutically acceptable salt thereof, wherein at least one of X¹, X², and X⁵ is —N(R)—, —C(O)—N(R)—, or —CH₂—.

36. The method of any one of claims 26-35, wherein X¹ is —C(O)—N(R)—.

37. The method of any one of claims 26-36, or a pharmaceutically acceptable salt thereof, wherein X² is —(O—CH₂—CH₂)ₙ—, —(CH₂—CH₂—O)ₙ—, or —C₁₋₅ alkyl-.

38. The method of any one of claims 26-37, wherein X³ is a bond, —C≡C—, —C₁₋₄ alkyl-, or —N(R)—.

39. The method of any one of claims 26-38, wherein X⁴ is a bond, —CH₂—, or N(R)—.

40. The compound or pharmaceutically acceptable salt of any one of claims 26-39, wherein X⁵ is a bond.

41. The method of any one of claims 26-40, wherein X¹ is —(O—CH₂—CH₂—CH₂)ₘ—, m is one, and X² is —C(O)—N(R)—.

42. The method of any one of claims 26-35 or 37-40, wherein X¹ is —CH₂—, —C(O)—,

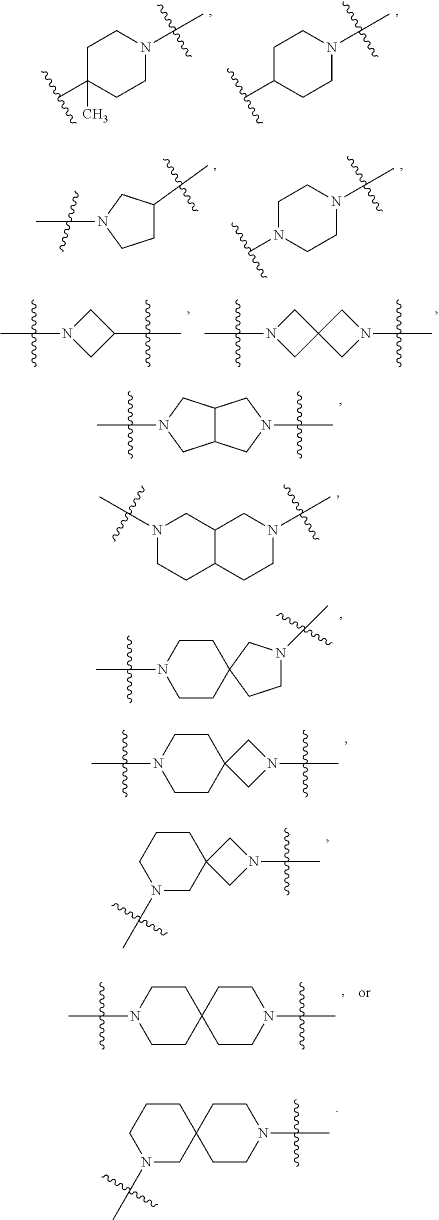

43. The method of any one of claims 26-37, 38-40, or 42, wherein X² is a bond, —C(O)—, —C₁₋₅ alkyl,

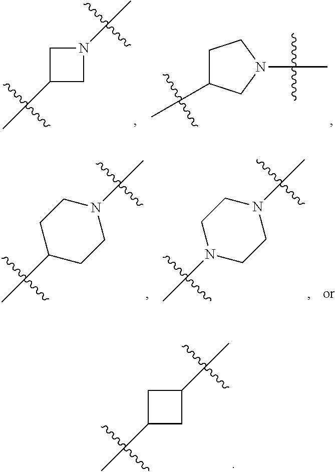 [SMILES: *C1CN(*)C1 ; *C1CCN(*)C1 ; *C1CCN(*)CC1 ; *N1CCN(*)CC1 ; *C1CC(*)C1]

44. The method of any one of claims 26-37 or 39-43, wherein X³ is bond, —C₁₋₄ alkyl-, 4-6 membered cycloalkyl, or —N(R)—.

45. The method of any one of claims 26-37 or 39-43, wherein X³ is a bond, —C₁₋₄ alkyl-, —NH—, 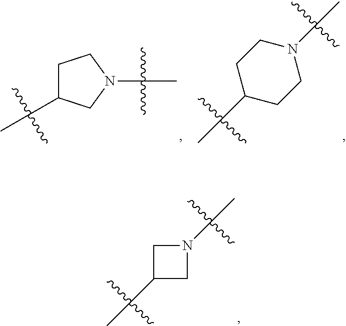 [SMILES: *C1CCN(*)C1 ; *C1CCN(*)CC1 ; *C1CN(*)C1]
or —C≡C—.

46. The method of any one of claims 26-38 or 40-44, wherein X⁴ is a bond, 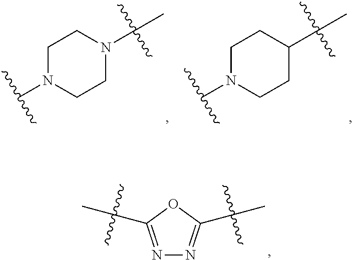 [SMILES: *N1CCN(*)CC1 ; *C1CCN(*)CC1 ; *c1nnc(*)o1]
—C₁₋₄ alkyl-, —CH₂—CH₂—N(R)—, or —N(R)—.

47. The method of any one of claims 26-39 or 41-46, wherein X⁵ is a bond, —C₁₋₄ alkyl-, —N(R)—, or —C(O)—N(R)—.

48. The method of any one of claims 26-47, wherein L is

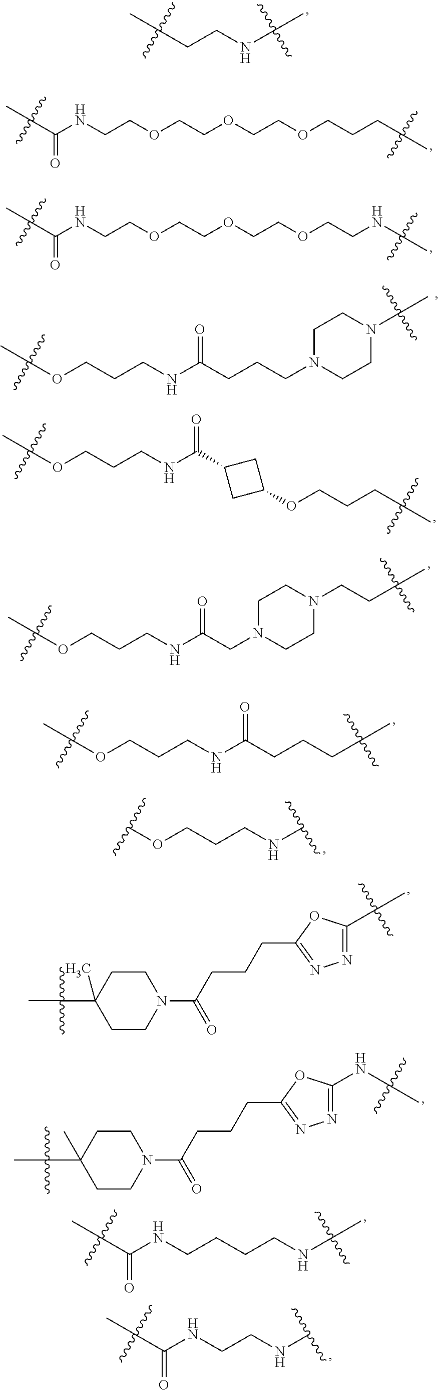

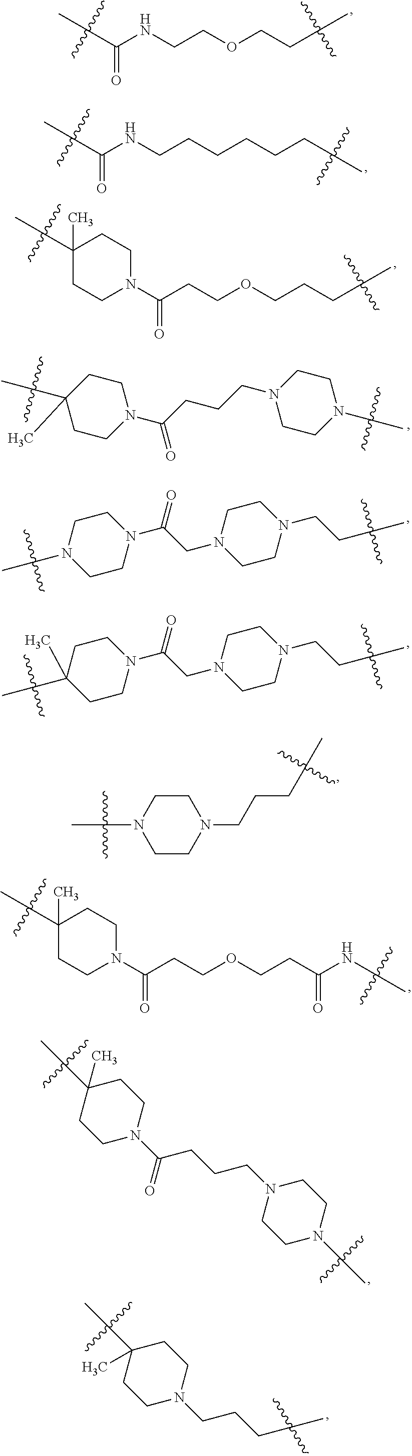

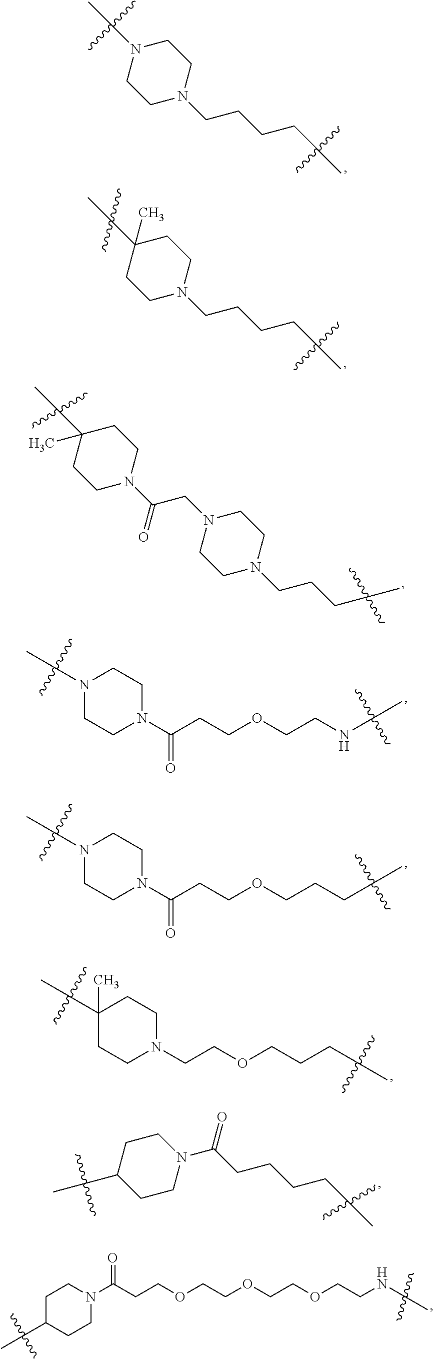

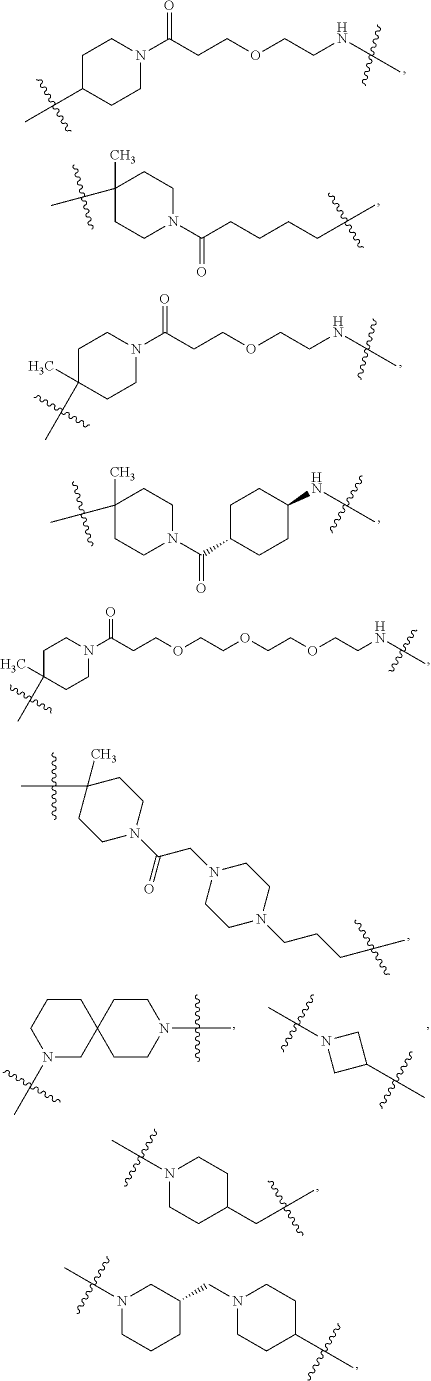

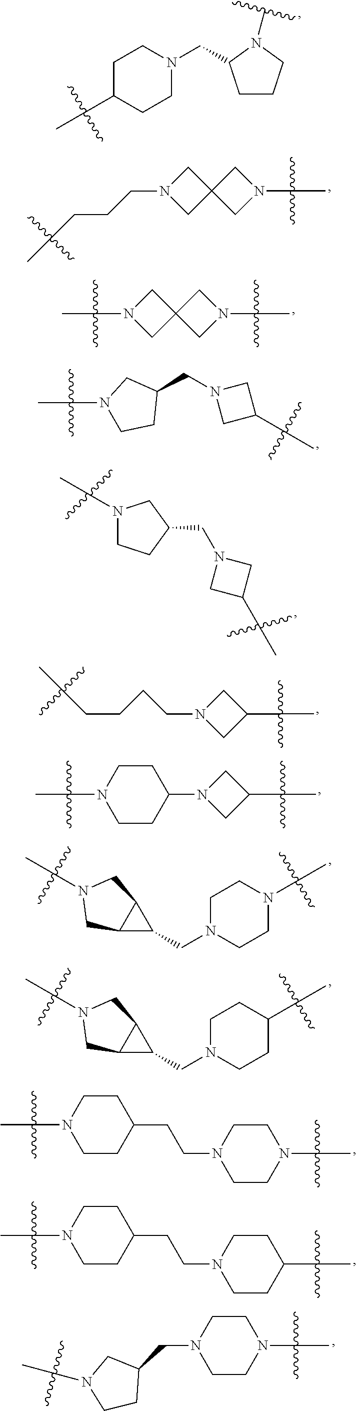

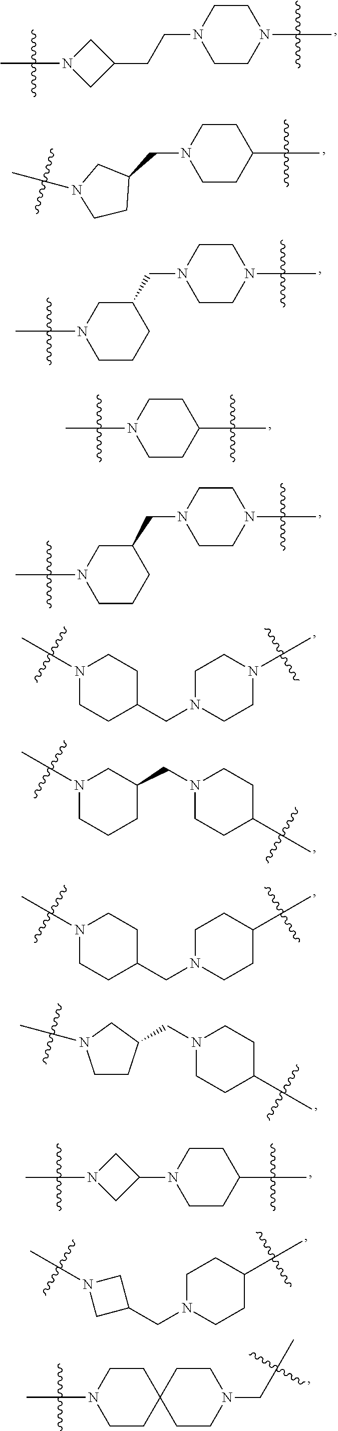

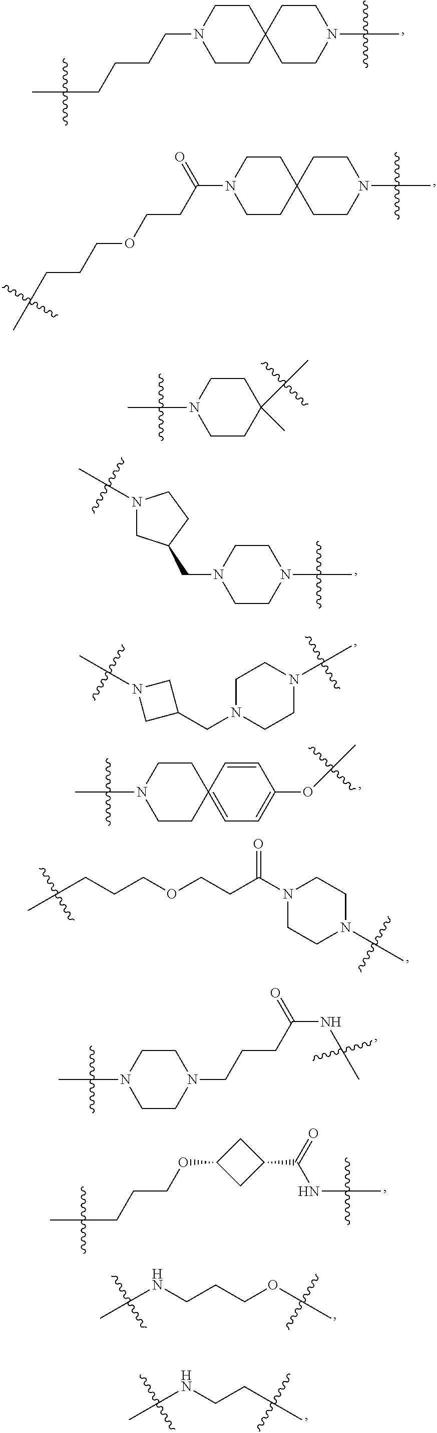

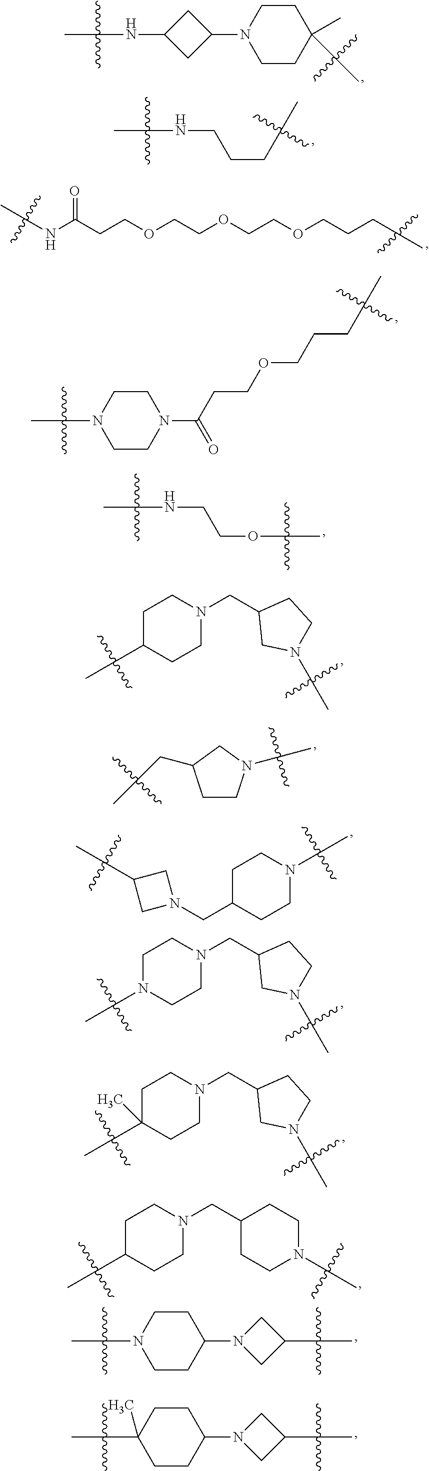

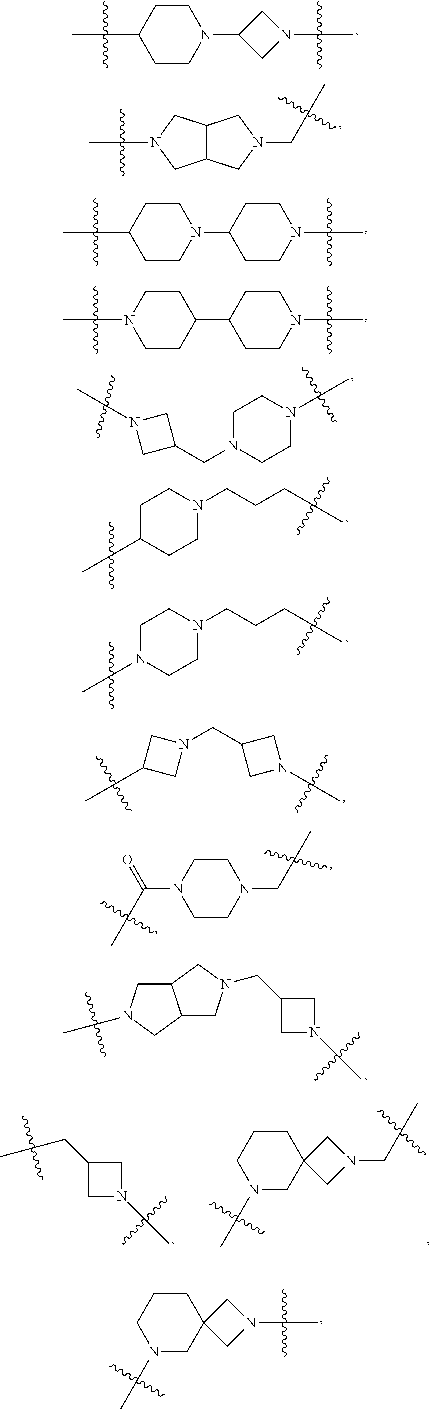

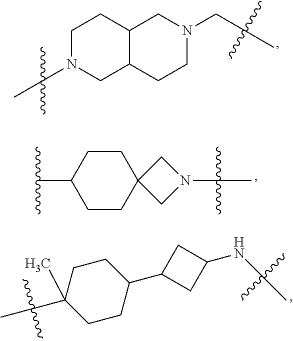

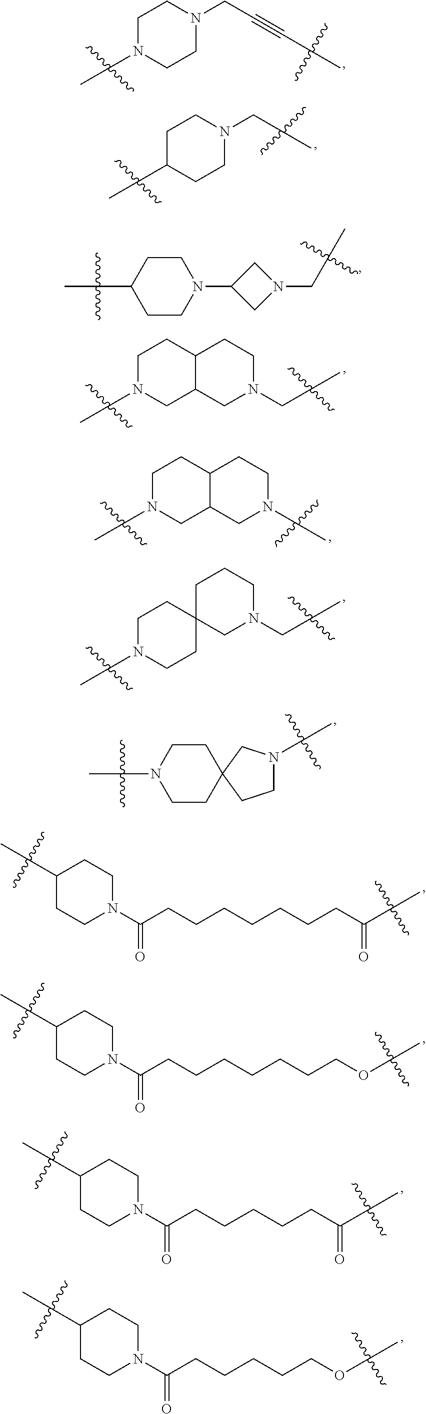

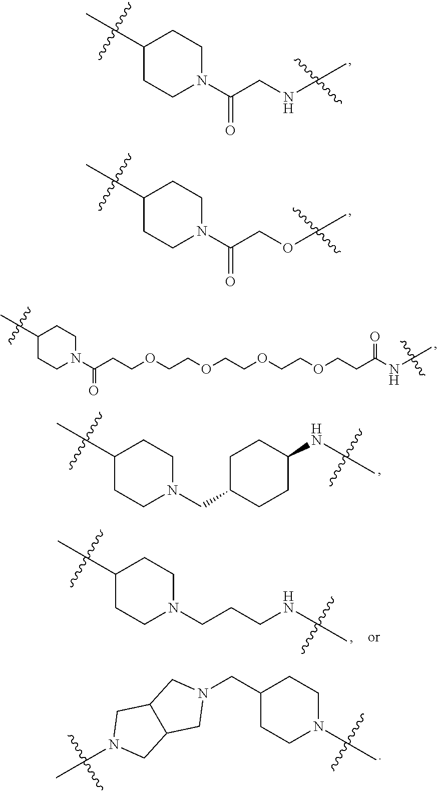 [SMILES: *NCC(=O)N1CCC(*)CC1 ; *OCC(=O)N1CCC(*)CC1 ; *NC(=O)CCOCCOCCOCCOCCC(=O)N1CCC(*)CC1 ; *N[C@H]1CC[C@H](CN2CCC(*)CC2)CC1 ; *NCCCN1CCC(*)CC1 ; *N1CCC(CN2CC3CN(*)CC3C2)CC1]

49. The method of any one of claims 26-48, wherein Y is

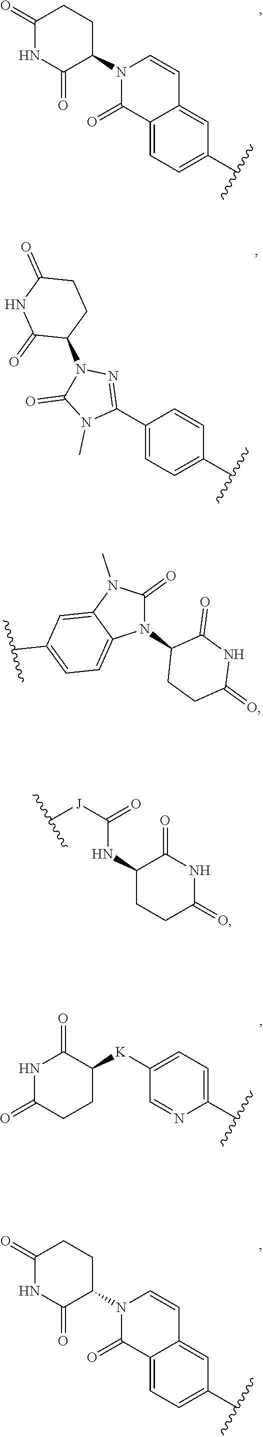

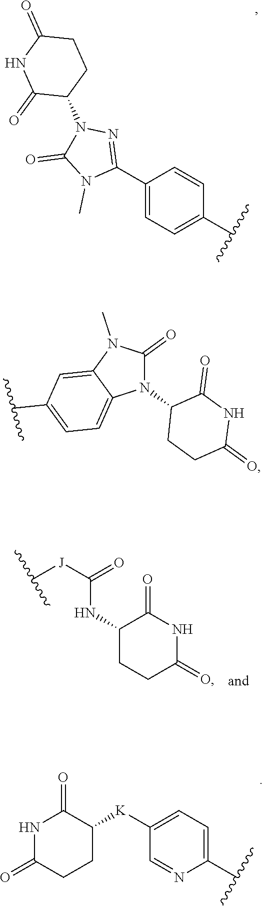

50. The method of any one of claims 26-49, wherein J is phenyl, naphthyl, pyridyl, pyrimidinyl, pyridizanyl, pyrazinyl, furanyl, pyrrolyl, imidazolyl, pyrazolyl, triazolyl, oxazolyl, or thiazolyl.

51. The method of any one of claims 26-50, wherein K is —CH₂—, —NH—, —NMe—, or —O—.

52. The method of claim 26, wherein the compound of Formula (A) is a compound of Formula (B) 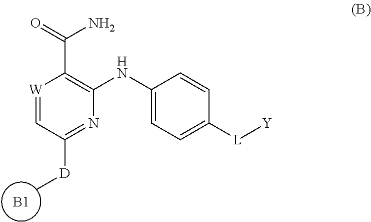
or a pharmaceutically acceptable salt thereof, wherein
W is CH or N;
D is a bond or —NH—;
Ring B1 is a 4-6 membered, fully saturated, partially unsaturated, or fully unsaturated monocyclic heterocycle or a 8-10 membered, fully saturated, spiro bicyclic heterocycle,
wherein Ring B1 has one to three heteroatoms independently selected from N, O, or S, and is optionally substituted with one to three groups selected from halo, —CH₃, —CF₃, —C(O)OH, —CH₂OH, or a five membered heterocycloalkyl optionally substituted with oxo and having one to two heteroatoms independently selected from N or O;
L is —X¹—X²—X³—;
X¹ is —C(O)—N(R) N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃;
X² is a bond, —(O—CH₂—CH₂)ₙ (CH₂—CH₂—O)ₙ, N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—,
—(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃;
each R is independently hydrogen or —C₁₋₃ alkyl; and
each of m, n, and p is independently an integer from one to three.

53. The method of claim 52, wherein Ring B1 is 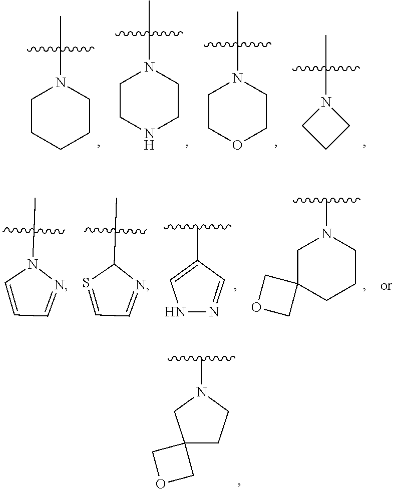
and Ring B1 is optionally substituted one to three groups selected from —CH₃, —CH₂OH, —C(O)OH, —CF₃, fluorine,

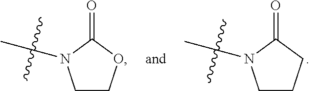 [SMILES: *N1CCOC1=O ; "and" ; *N1CCCC1=O]

54. The method of either of claims 52 or 53, wherein Ring B1 is

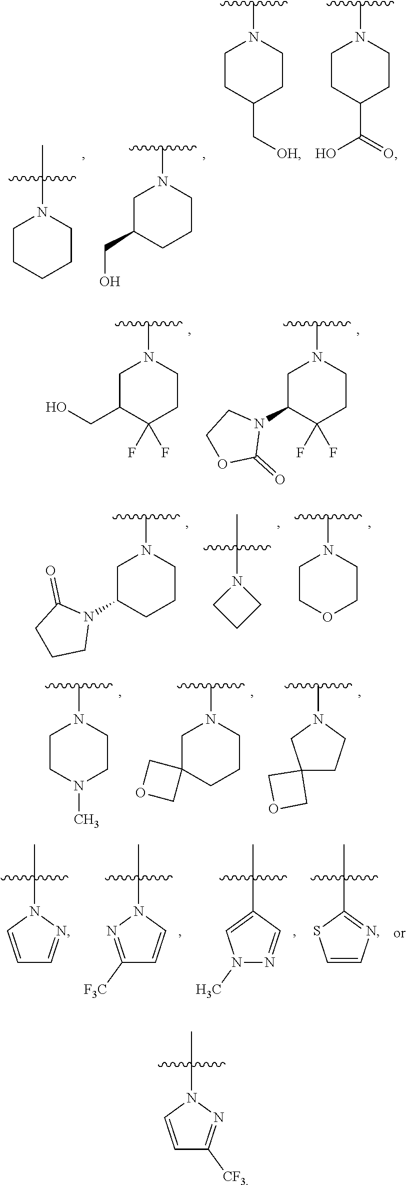

55. The method of any one of claims 52-54 wherein Ring B1 is

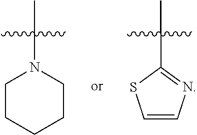 [SMILES: *N1CCCCC1 ; "or" ; *c1nccs1]

56. The method of any one of claims 52-55, wherein X¹ is

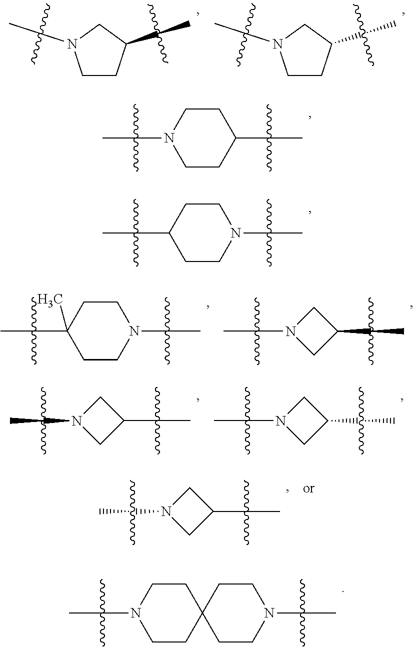

57. The method of any one of claims 52-56, wherein X² is a bond, —C₁₋₅ alkyl-, 4-6 membered monocyclic cycloalkyl, or 4-6 membered monocyclic heterocycloalkyl having one to two heteroatoms independently selected from N, O, or S.

58. The method of any one of claims 52-57, wherein X² is a bond, —C₁₋₃ alkyl-, —C(O)—,

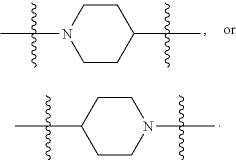

59. The method of any one of claims 52-58, wherein X³ is a bond, —C₁₋₄ alkyl-, —N(R)—, —(O—CH₂—CH₂)ₚ—, —(CH₂—CH₂—O)ₚ—, or a 4-6 membered heterocycloalkyl having one to two heteroatoms independently selected from N, O, or S, wherein the heterocycloalkyl is optionally substituted with —CH₃.

60. The method of any one of claims 52-59, wherein X³ is a bond,

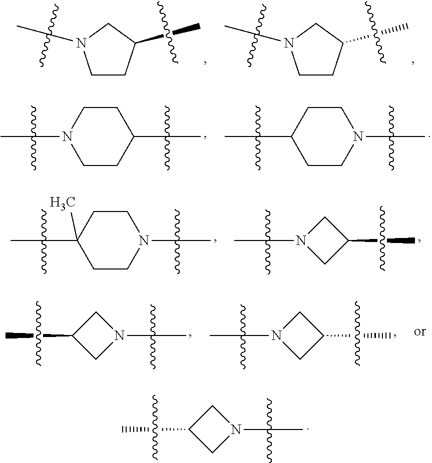

61. The method of any one of claims 52-60, wherein L is

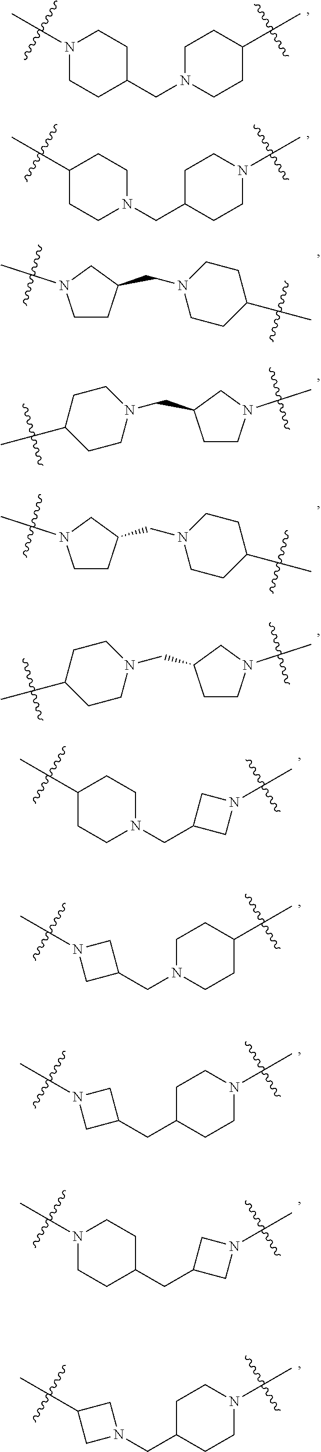

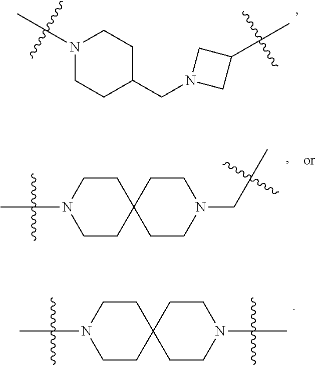 [SMILES: *C1CN(CC2CCN(*)CC2)C1 ; *CN1CCC2(CCN(*)CC2)CC1 ; *N1CCC2(CC1)CCN(*)CC2]

62. The method of any one of claims 52-61, wherein W is N and D is a bond.

63. The method of claim 26, wherein the compound of Formula (A) is a compound of Formula (C) 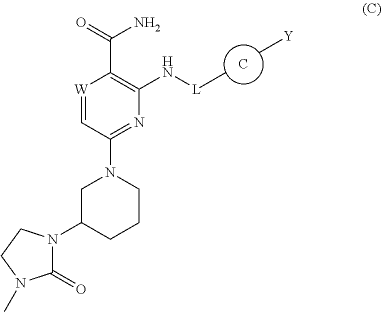
or a pharmaceutically acceptable salt thereof, wherein
W is CH or N;
Ring C is phenyl or a saturated, partially unsaturated, or fully unsaturated 5-6 membered monocyclic heterocycle having one to two heteroatoms independently selected from N, O, or S, wherein each of the phenyl and heterocycle of Ring C is optionally substituted;
L is —X¹—X²—X³—;
X¹ is —C(O)—N(R), N(R)—C(O), (O—CH₂—CH₂)ₘ 0-(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S,
wherein each of the bicyclic heterocycloalkyl and the monocyclic heterocycloalkyl of X¹ is optionally substituted with —CH₃;
X² is a bond, —(O—CH₂—CH₂)ₙ (CH₂—CH₂—O)ₙ, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃;
each R is independently hydrogen or —C₁₋₃ alkyl; and
each of m, n, and p is independently an integer from one to three.

64. The method of claim 63, wherein Ring C is

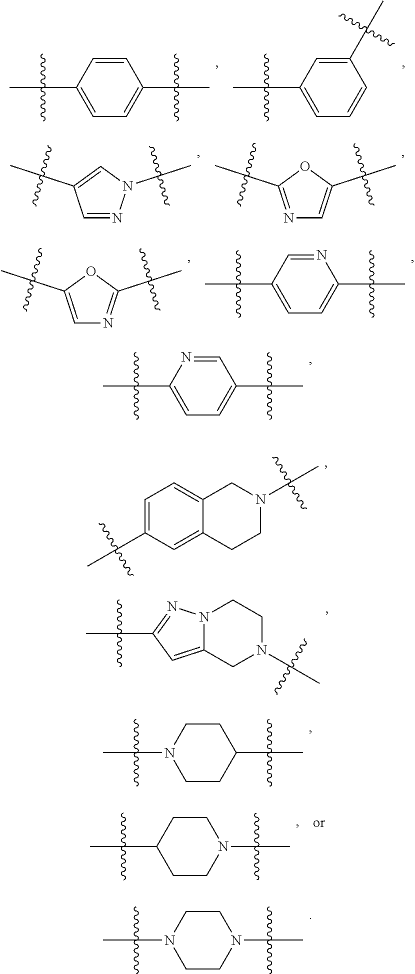

65. The method of either of claims 63 or 64, wherein Ring C is

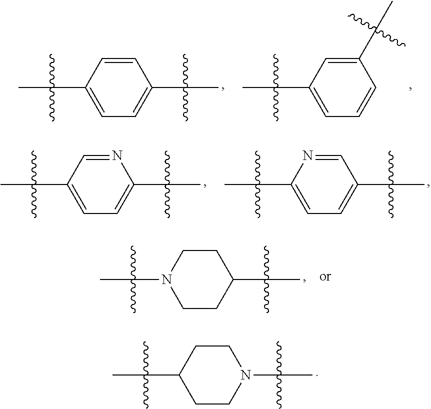

66. The method of any one of claims 63-65, wherein X¹ is a 4-6 membered monocyclic heterocycloalkyl having one to two heteroatoms independently selected from N, O, or S.

67. The method of any one of claims 63-66 wherein X¹ is

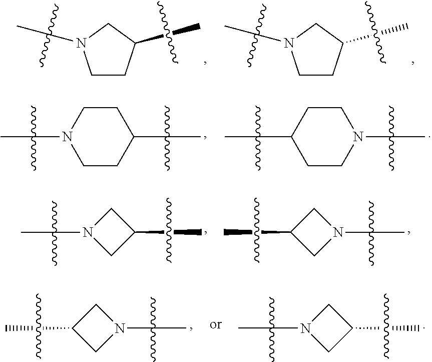

68. The method of any one of claims 63-67, wherein X² is a bond, —C₁₋₅ alkyl-, 4-6 membered monocyclic cycloalkyl, or 4-6 membered monocyclic heterocycloalkyl having one to two heteroatoms independently selected from N, O, or S.

69. The compound or pharmaceutically acceptable salt of any one of claims 63-68, wherein X² is a bond or —C₁₋₃ alkyl-.

70. The method of any one of claims 63-69, wherein X³ is a 4-6 membered cycloalkyl, —N(R)—, or a 4-6 membered heterocycloalkyl having one to two heteroatoms independently selected from N, O, or S, wherein the heterocycloalkyl is optionally substituted with —CH₃.

71. The method of any one of claims 63-70, wherein X³ is

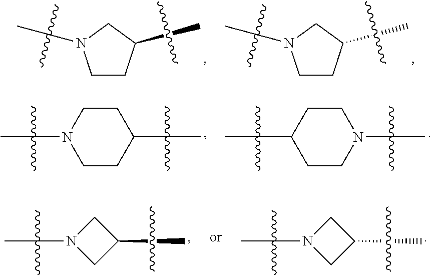

72. The method of any one of claims 63-71, wherein L is

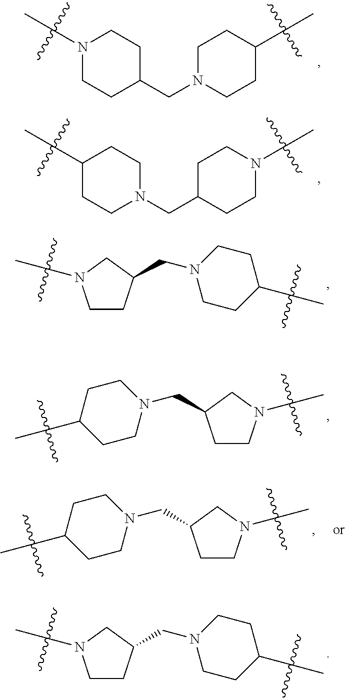

73. The method of claim 26, wherein the compound of Formula (A) is a compound of Formula (D) 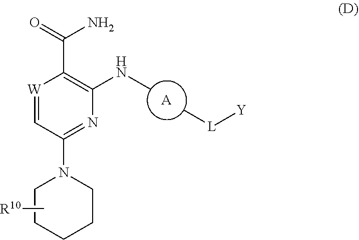
or a pharmaceutically acceptable salt thereof, wherein
W is CH or N;
Ring A is 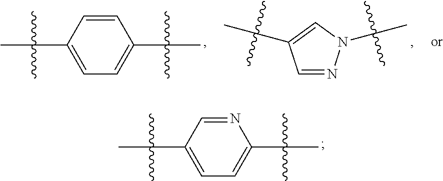 [SMILES: *c1ccc(*)cc1 ; *c1cnn(*)c1 ; *c1ccc(*)nc1]
L is —X¹—X²—X³—;
X¹ is —C₁₋₅ alkyl- or 4-6 membered monocyclic heterocycloalkyl having one to two heteroatoms independently selected from N, O, or S, wherein the monocyclic heterocycloalkyl of X¹ is optionally substituted with —CH₃;
X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is optionally substituted with —CH₃;
X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃; and
R¹⁰ is halo, —C₁₋₅ alkyl, 3-6 membered cycloalkyl, 5-6 membered heterocycloalkyl, —CN, —OH, —CF₃, —C(O)OH, —CH₂OH, —CH₂CH₂OH,

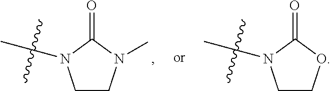

74. The method of claim 73, wherein the compound of Formula (D) is a compound of Formula (D-1) 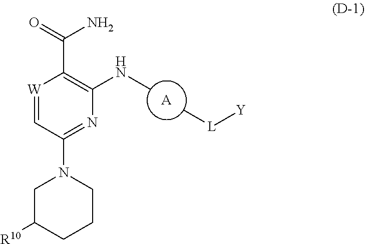
or a pharmaceutically acceptable salt thereof, wherein
W is CH or N;
Ring A is 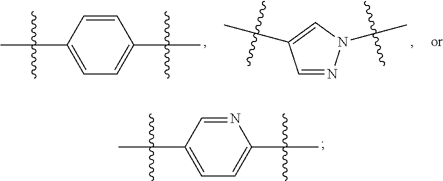 [SMILES: *c1ccc(*)cc1 ; *c1cnn(*)c1 ; *c1ccc(*)nc1]
L is —X¹—X²—X³—;
X¹ is —C₁₋₅ alkyl- or 4-6 membered monocyclic heterocycloalkyl having one to two heteroatoms independently selected from N, O, or S, wherein the monocyclic heterocycloalkyl of X¹ is optionally substituted with —CH₃;
X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is optionally substituted with —CH₃;
X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃; and
R¹⁰ is

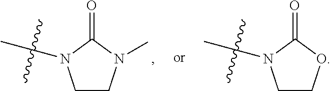

75. The method of claim 73, wherein the compound of Formula (D) is a compound of Formula (D-2) 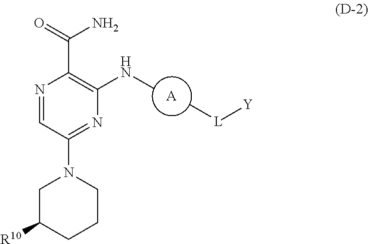
or a pharmaceutically acceptable salt thereof.

76. The method any one of claims 73-75, wherein Ring A is

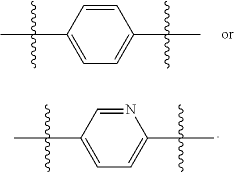 [SMILES: *c1ccc(*)cc1 ; *c1ccc(*)nc1]

77. The method of any one of claims 73-76, wherein X¹ is a 4-6 membered monocyclic heterocycloalkyl having one to two heteroatoms independently selected from N, O, or S, wherein the monocyclic heterocycloalkyl of X¹ is optionally substituted with —CH₃.

78. The method of any one of claims 73-77, wherein X¹ is

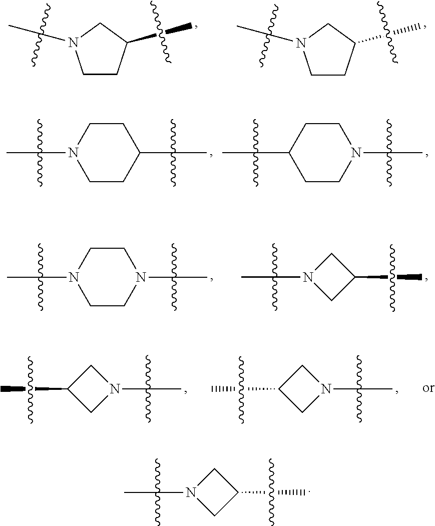

79. The method of any one of claims 73-78, wherein X² is a bond, —C₁₋₅ alkyl-, 4-6 membered monocyclic cycloalkyl, or 4-6 membered monocyclic heterocycloalkyl having one to two heteroatoms independently selected from N, O, or S.

80. The method of any one of claims 73-79, wherein X² is a bond or —C₁₋₄ alkyl-.

81. The method of any one of claims 73-80, wherein X³ is a bond, a 4-6 membered monocyclic cycloalkyl, or 4-6 membered monocyclic heterocycloalkyl having one to two heteroatoms independently selected from N, O, or S.

82. The method of any one of claims 73-81, wherein X³ is,

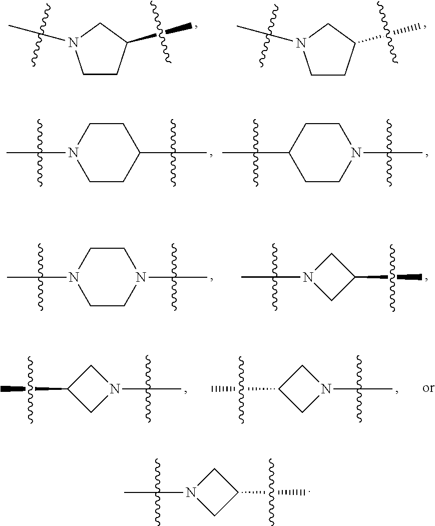

83. The method of any one of claims 73-82, wherein L is

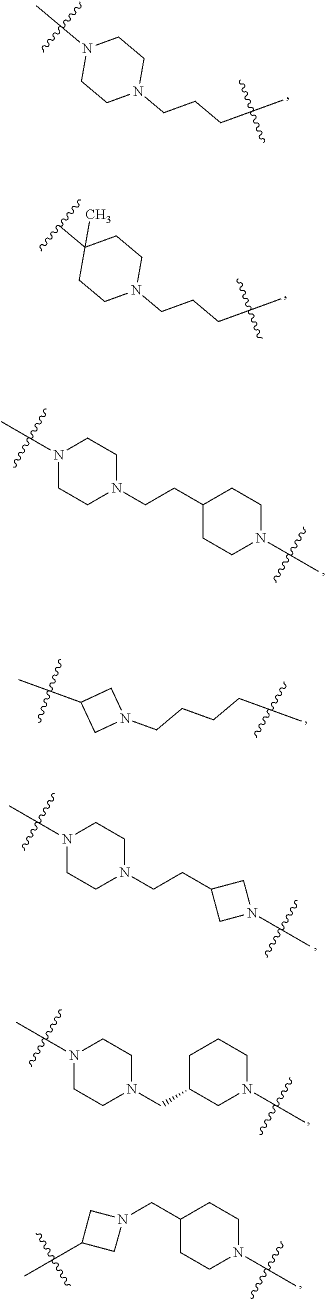

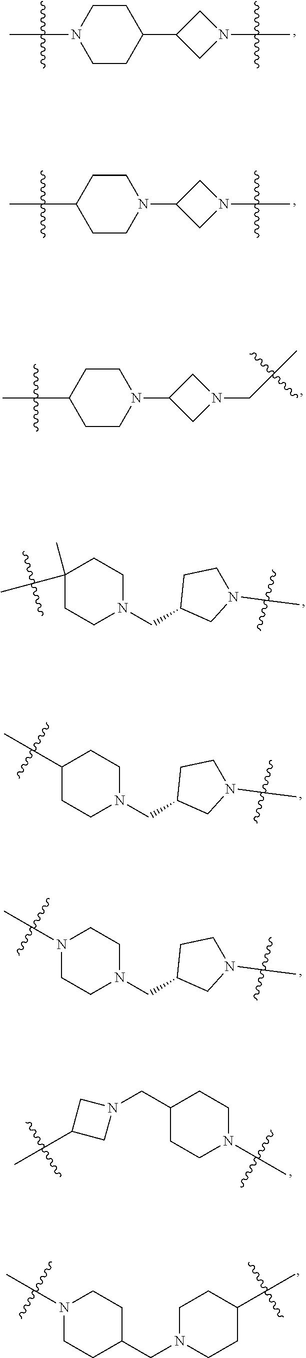

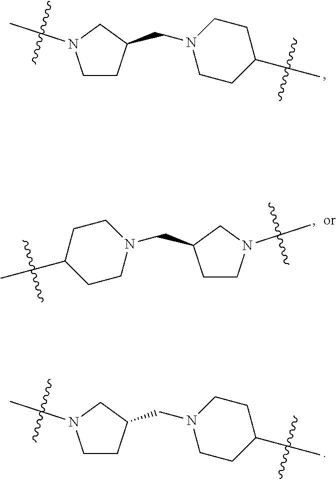 [SMILES: *C1CCN(C[C@H]2CCN(*)C2)CC1 ; *C1CCN(C[C@@H]2CCN(*)C2)CC1 ; *C1CCN(C[C@@H]2CCN(*)C2)CC1]

84. The method of any one of claims 73-83, wherein R¹⁰ is

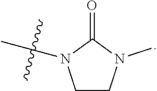 [SMILES: *N1CCN(C)C1=O]

85. The method of any one of claims 72-82, wherein R¹⁰ is

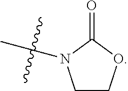 [SMILES: *N1CCOC1=O]

86. The method of claim 26, wherein the compound of Formula (A) is a compound of Formula (E) 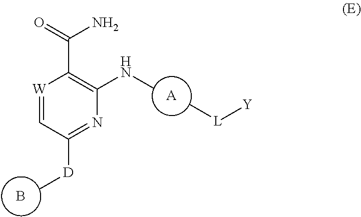
or a pharmaceutically acceptable salt thereof, wherein
D is a bond or —NH—;
W is N or CH;
Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S;
Ring B is an optionally substituted 5-6 membered saturated, partially unsaturated, or fully unsaturated monocyclic heterocycle, or an optionally substituted 8-10 membered spiro bicyclic heterocycle, wherein Ring B has one to three heteroatoms independently selected from N, O, or S;
L is —X¹—X²—X³—X⁴—X⁵—;
X¹ is a bond, —C(O)—N(R) N(R)—C(O) (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃;
X² is a bond, —(O—CH₂—CH₂)ₙ (CH₂—CH₂—O)ₙ, N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S;
X⁵ is a bond, —N(R)—, or
each R is independently hydrogen or —C₁₋₃ alkyl;
each of m, n, and p is independently an integer from one to three; and
wherein at least one of X¹, X², X³, X⁴, and X⁵ has a nitrogen atom, and Y is directly bonded to L at a nitrogen atom of X¹, X², X³, X⁴, or X⁵.

87. The method of claim 86, wherein Ring B is 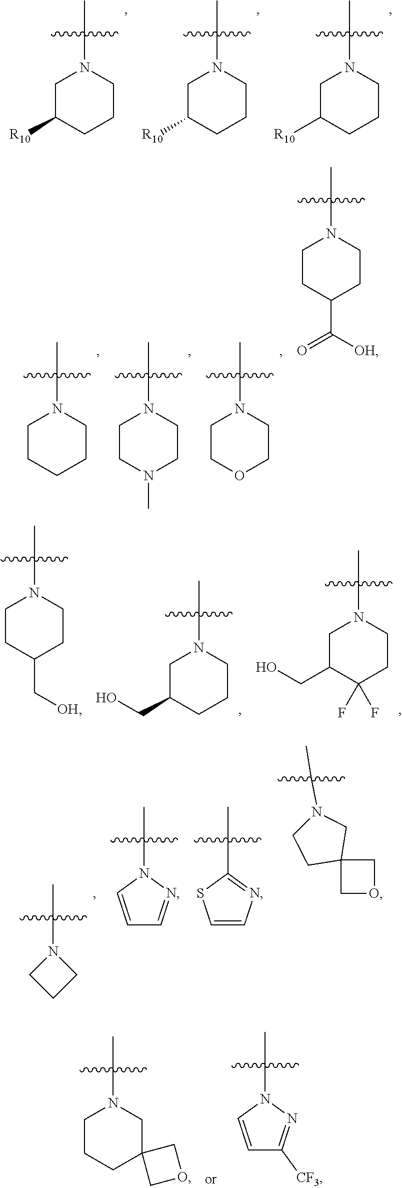
wherein R¹⁰ is 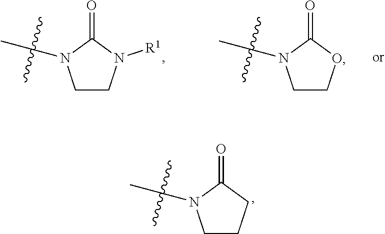
and wherein R¹ is a C₁₋₄ alkyl group.

88. The method either of claims 86 or 87, wherein Ring B is 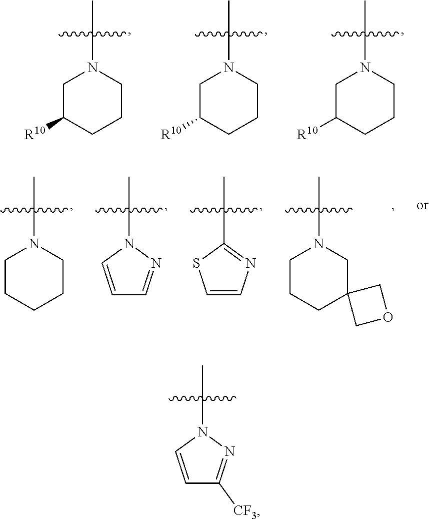
wherein R¹⁰ is

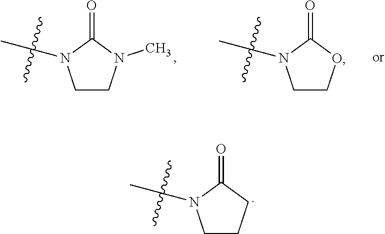

89. The method of any one of claims 86-88, wherein Ring B is

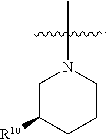

90. The method of any one of claims 86-89, wherein R¹⁰ is

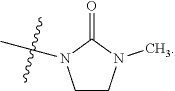 [SMILES: *N1CCN(C)C1=O]

91. The method of any one of claims 86-90, wherein Ring A is

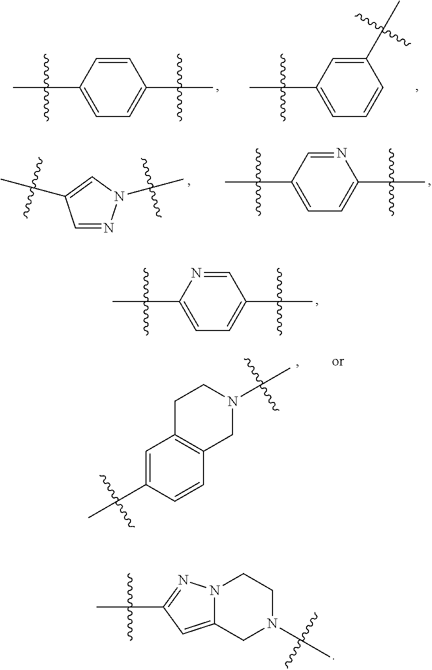

92. The method of any one of claims 86-91, wherein X⁵ is —N(R)—.

93. The method of any one of claims 86-92, wherein X⁵ is —C(O)—N(R)—.

94. The method of any one of claims 86-93, wherein X⁵ is a bond.

95. The method of any one of claims 86-94, wherein L is

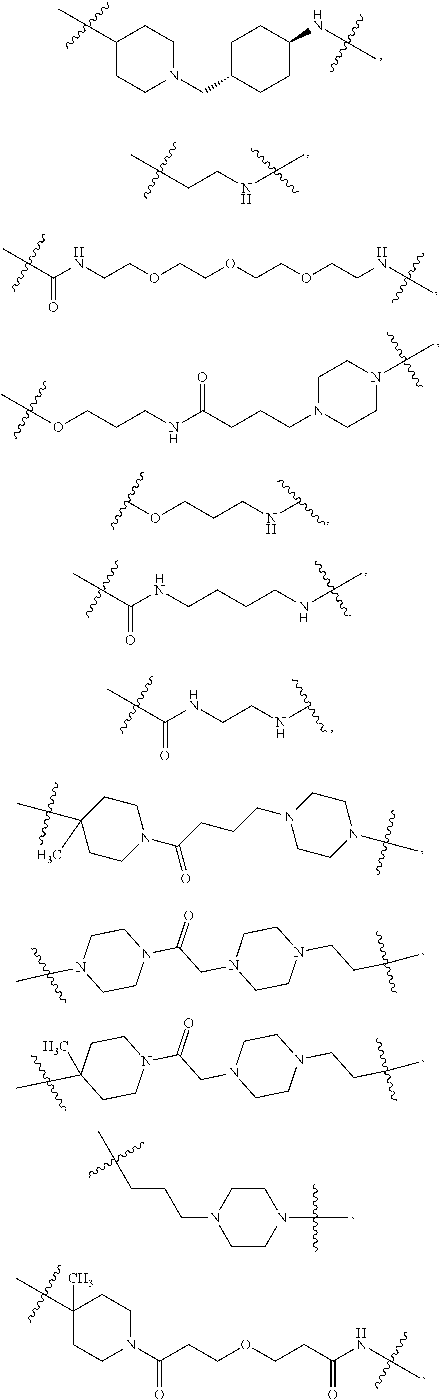

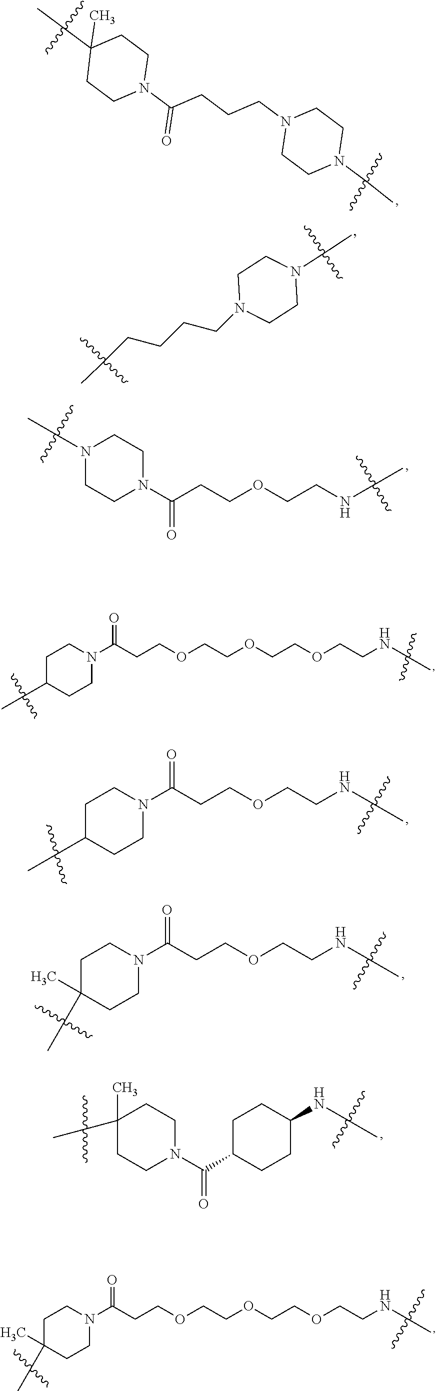

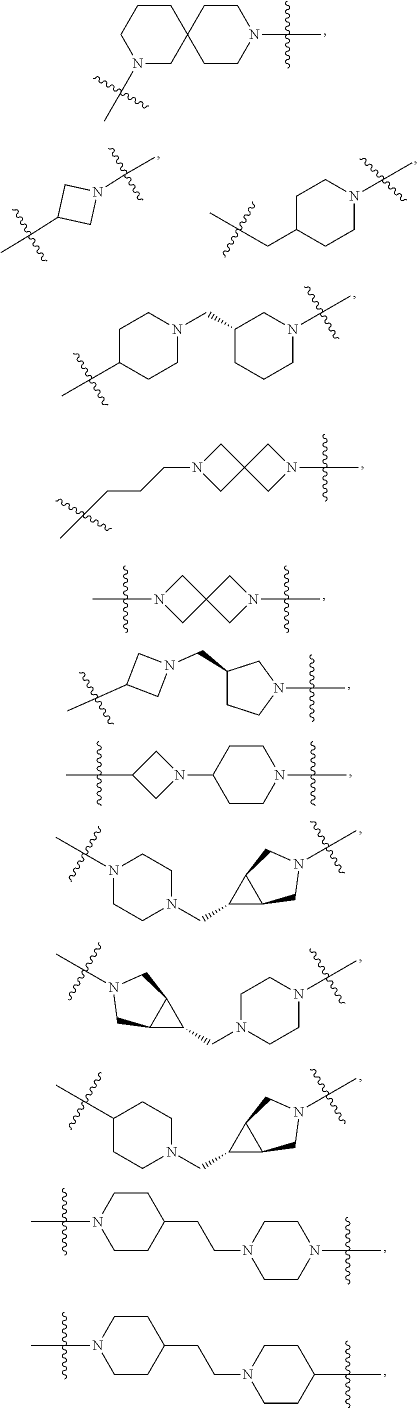

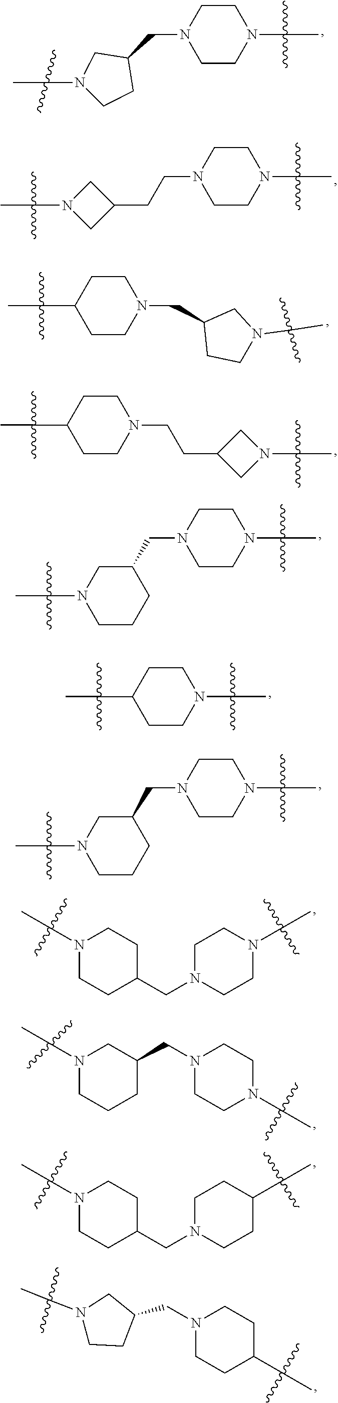

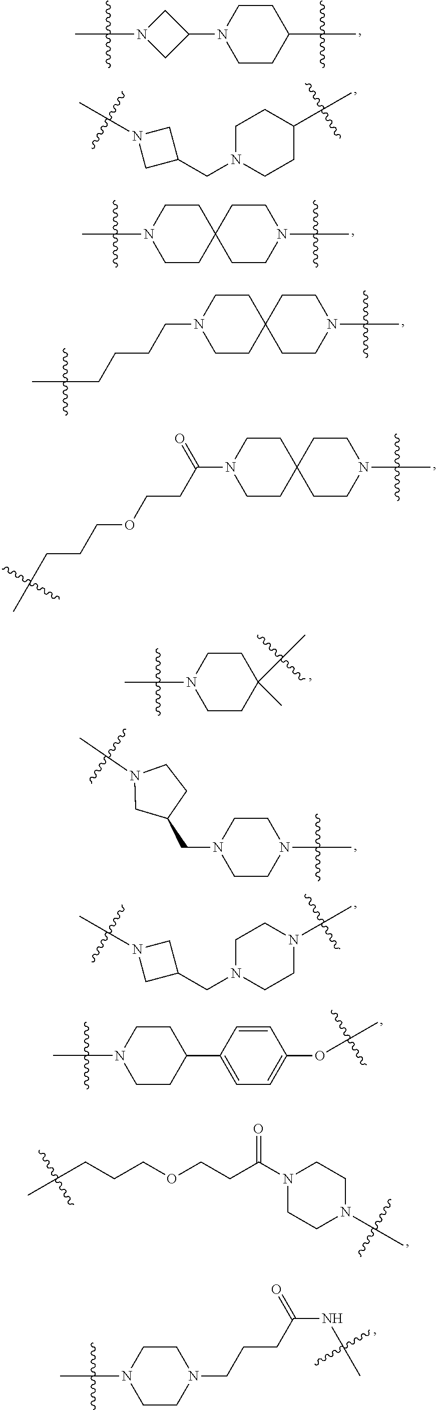

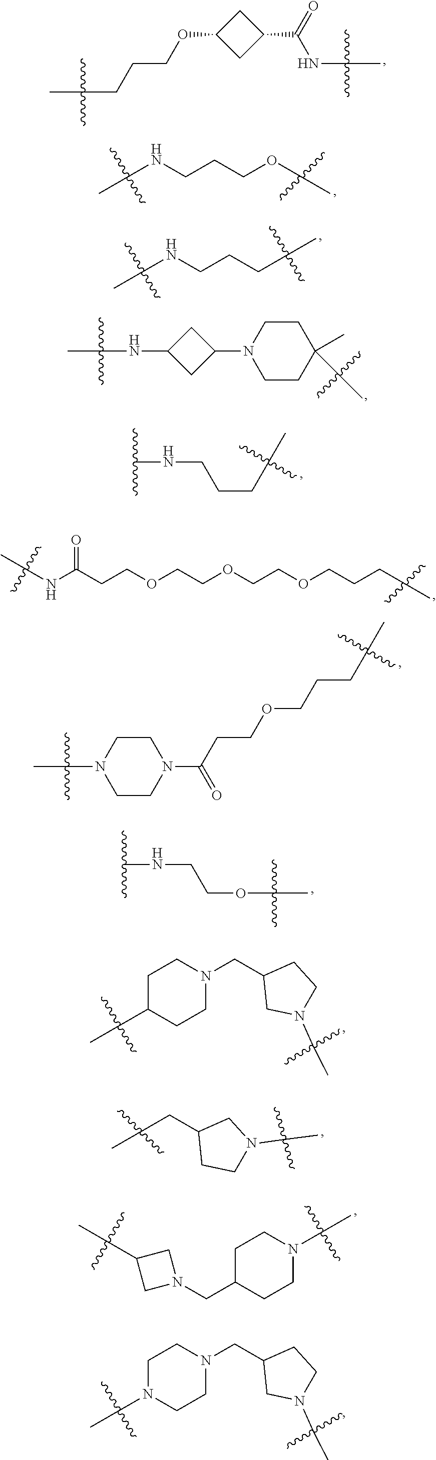

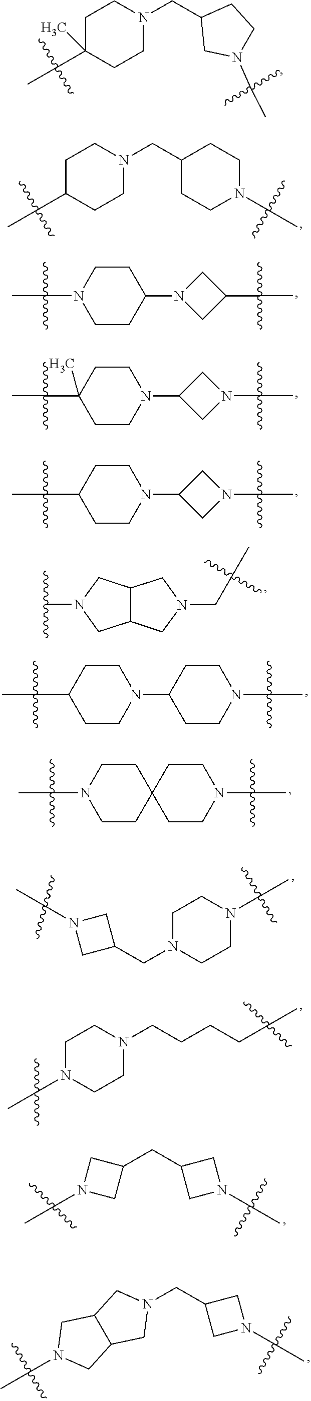

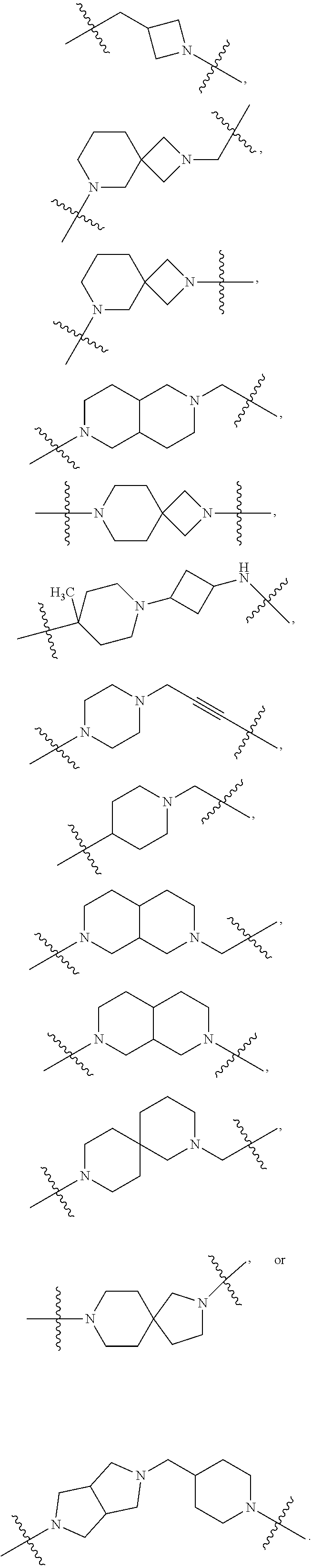

96. The method of any one of claims 86-95, wherein Y is

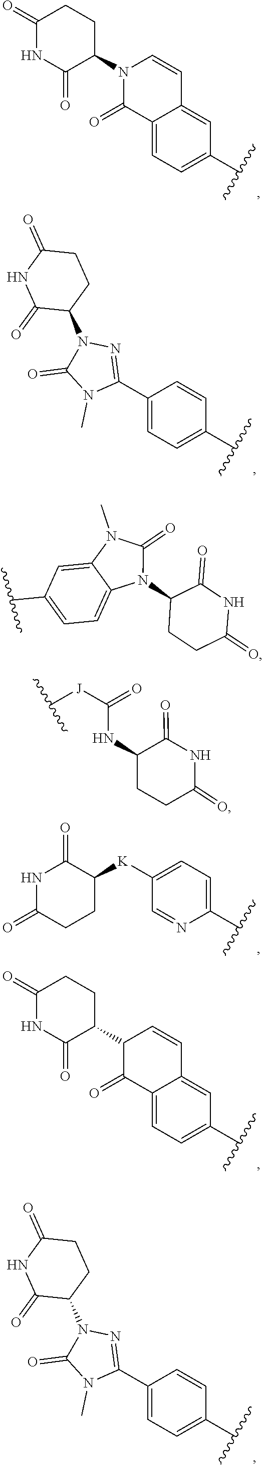

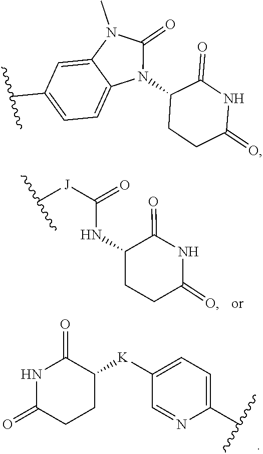

97. The method of claim 26, wherein the compound of Formula (A) is a compound of Formula (F) 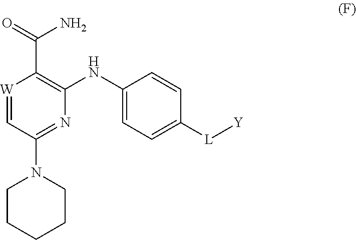
or a pharmaceutically acceptable salt thereof, wherein
W is CH or N;
L is —X¹—X²—X³—;
X¹ is —C(O)—N(R), N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃;
X² is a bond, —C₁₋₅ alkyl-, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃;
each R is independently hydrogen or —C₁₋₃ alkyl; and
each of m, n, and p is independently an integer from one to three.

98. The method of claim 97, wherein W is N.

99. The method of either of claims 97 or 98, wherein Y is

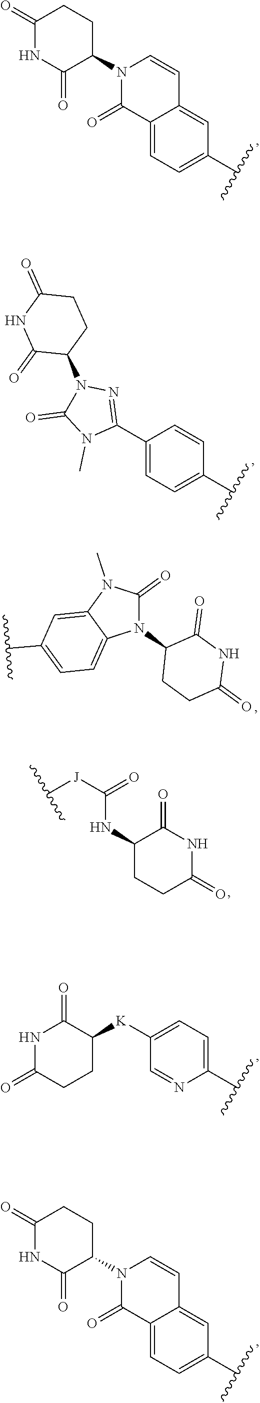

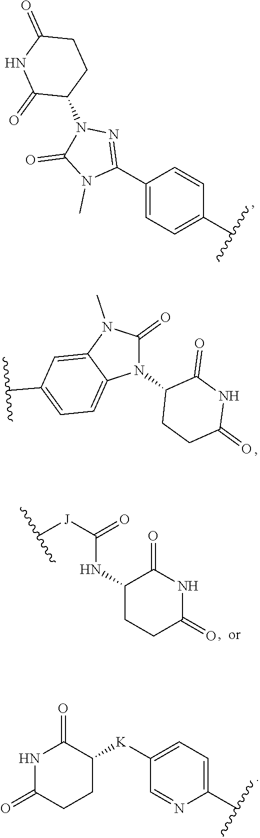

100. The method of any one of claims 97-99, wherein X¹ is a 4-6 membered monocyclic heterocycloalkyl having one to two heteroatoms independently selected from N, O, or S, wherein each of the monocyclic heterocycloalkyl of X¹ is optionally substituted with —CH₃.

101. The method of any one of claims 97-100, wherein X¹ is

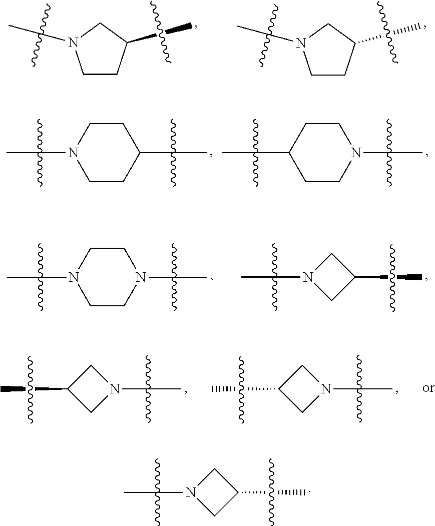

102. The method of any one of claims 97-101, wherein X¹ is

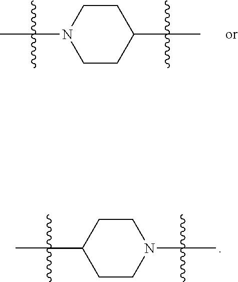 [SMILES: *C1CCN(*)CC1 ; *C1CCN(*)CC1]

103. The method of any one of claims 97-102, wherein X² is a bond or —C₁₋₅ alkyl-.

104. The method of any one of claims 97-103, wherein X³ is a 4-6 membered monocyclic heterocycloalkyl having one to two heteroatoms independently selected from N, O, or S.

105. The method of any one of claims 97-104, wherein X³ is

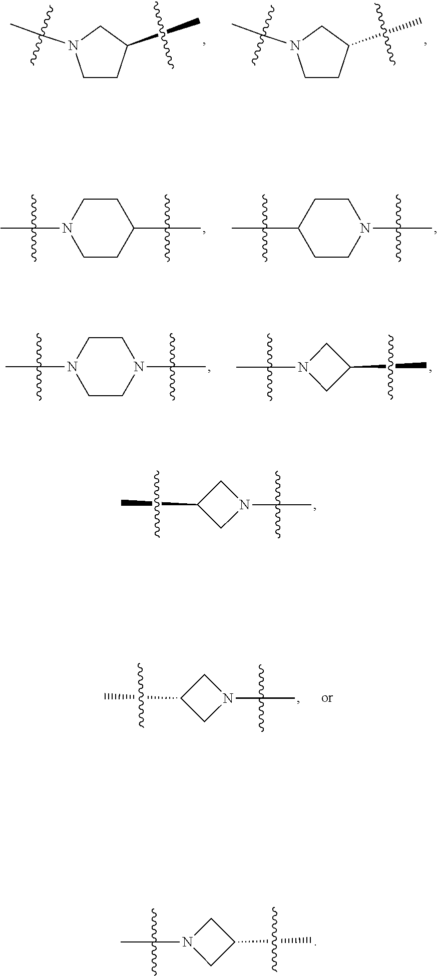

106. The method of any one of claims 97-105, wherein X³ is

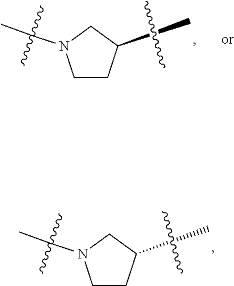

107. The method of any one of claims 97-106, wherein L is

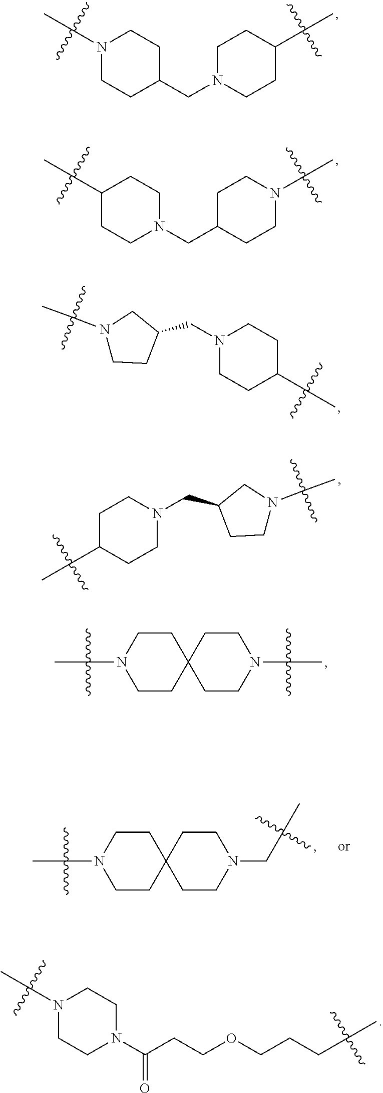

108. The method of claim 26, wherein the compound of Formula (A) is a compound of Formula (G) 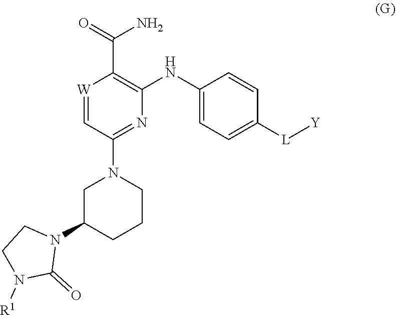
or a pharmaceutically acceptable salt thereof.

109. The method of claim 108, wherein R¹ is methyl.

110. The method of either of claims 108 or 109, wherein Y is

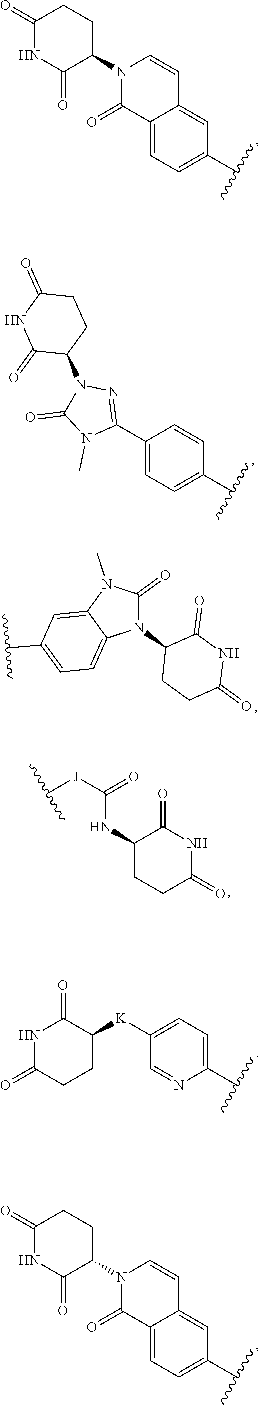

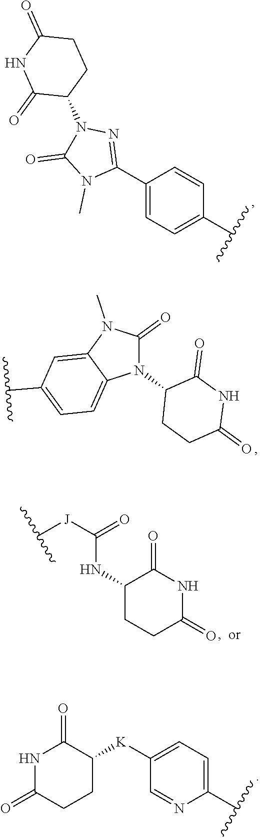

111. The method of any one of claims 108-110, wherein W is N.

112. The method of claim 26, wherein the compound of Formula (A) is a compound of Formula (J) 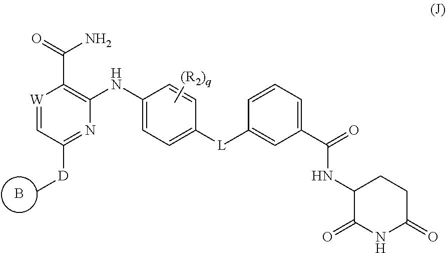
or a pharmaceutically acceptable salt thereof.

113. The method of claim 26, wherein the compound of Formula (A) is a compound of Formula (K) 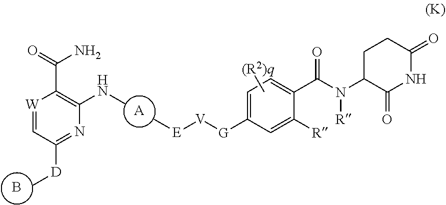
or a pharmaceutically acceptable salt thereof, wherein
Ring A is 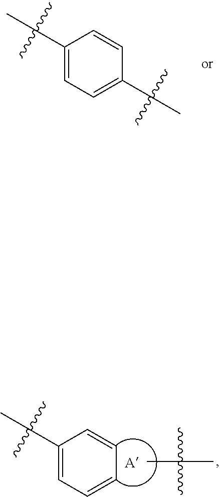
wherein Ring A is optionally and independently substituted with up to three substituents selected from halo, CN, carboxyl, NH₂, and optionally substituted C₁₋₆ alkyl;
V is a bond or —CH₂—; and
E and G are each independently a 5-6 membered heterocycloalkyl, wherein each heterocycloalkyl contains at least one nitrogen atom.

114. The method of claim 113, wherein D is a bond and W is a nitrogen atom.

115. The method of claim 26, wherein the compound of Formula (A) is a compound of Formula (M) 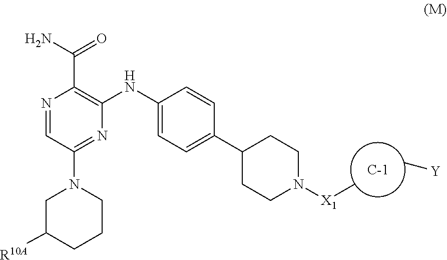
or a pharmaceutically acceptable salt thereof, wherein
R^{10A} is hydrogen, 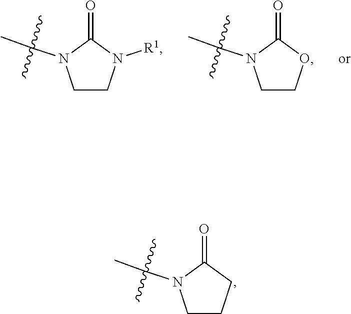
wherein
R¹ is C₁₋₄ alkyl;
X¹ is —C₁₋₅ alkyl-;
Ring C-1 is a 5-6 membered heterocycloalkyl having one nitrogen atom; and
Y is

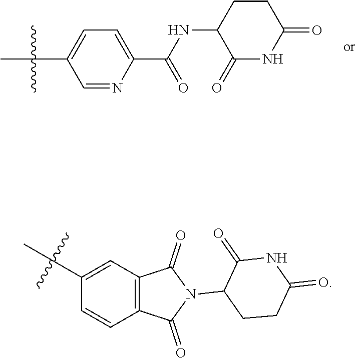 [SMILES: *c1ccc(C(=O)NC2CCC(=O)NC2=O)nc1 ; *c1ccc2c(c1)C(=O)N(C1CCC(=O)NC1=O)C2=O]

116. The method of claim 115, wherein R^{10A} is hydrogen or

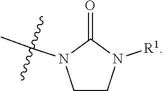

117. The method of either of claims 115 or 116, wherein R^{10A} is 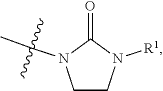
and R¹ is methyl, ethyl, propyl, iso-propyl, butyl, sec-butyl, or iso-butyl.

118. The method of any one of claims 115-117, wherein R¹ is methyl.

119. The method of any one of claims 115-118, wherein X¹ is —CH₂—, —CH₂CH₂—, or —CH₂CH₂CH₂—.

120. The method of any one of claims 115-119, wherein X¹ is —CH₂—.

121. The method of any one of claims 115-120, wherein Ring C-1 is

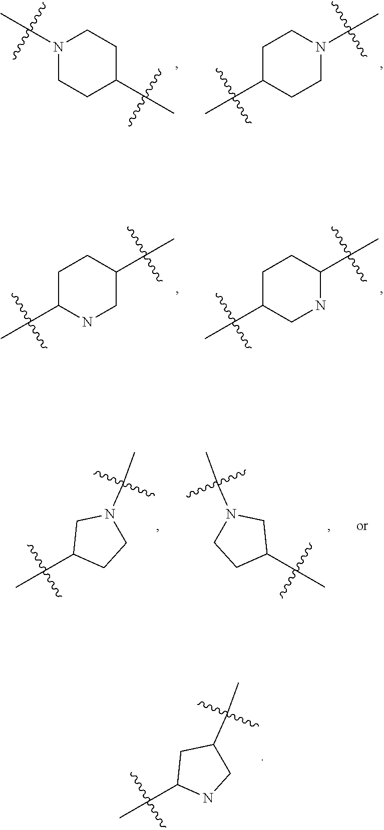

122. The method of any one of claims 115-121, wherein Ring C-1 is

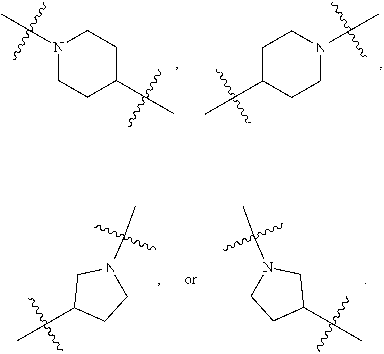 [SMILES: *C1CCN(*)CC1 ; *C1CCN(*)CC1 ; *C1CCN(*)C1 ; *C1CCN(*)C1]

123. The method of claim 1 wherein the compound is selected from Table 1, or a pharmaceutically acceptable salt thereof.

124. The method of any of the previous claims, wherein the compound is administered in the form of a pharmaceutical composition comprising the compound or pharmaceutically acceptable salt thereof and a pharmaceutically acceptable carrier, vehicle, or adjuvant.

125. A compound s elected from the group consisting of 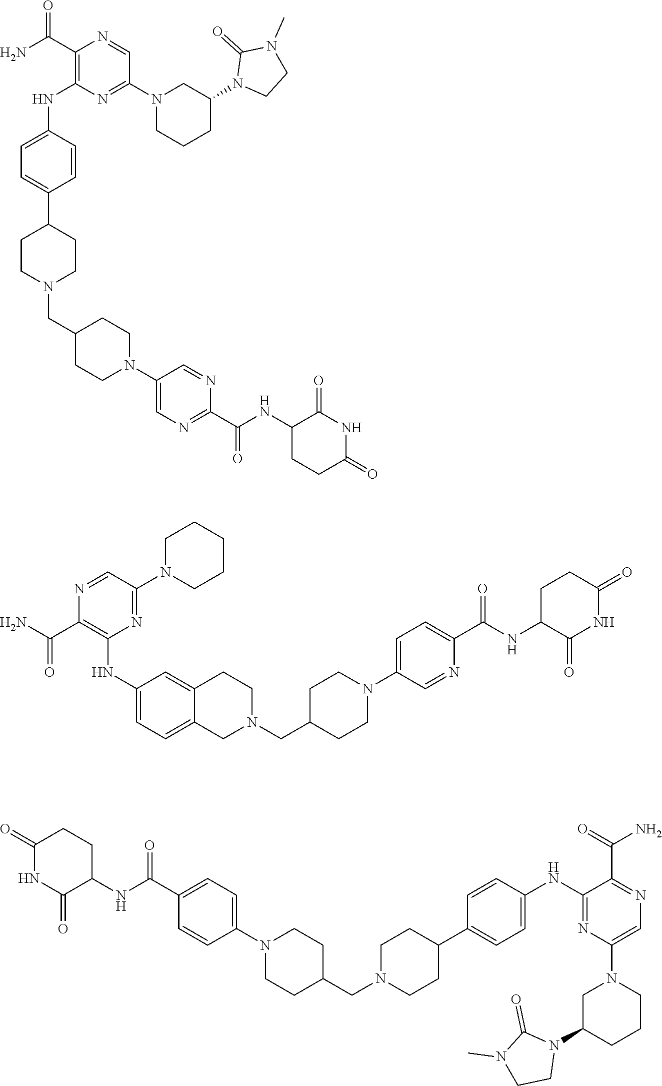 [SMILES: CN1CCN([C@@H]2CCCN(c3cnc(C(N)=O)c(Nc4ccc(C5CCN(CC6CCN(c7cnc(C(=O)NC8CCC(=O)NC8=O)nc7)CC6)CC5)cc4)n3)C2)C1=O ; NC(=O)c1ncc(N2CCCCC2)nc1Nc1ccc2c(c1)CCN(CC1CCN(c3ccc(C(=O)NC4CCC(=O)NC4=O)nc3)CC1)C2 ; CN1CCN([C@@H]2CCCN(c3cnc(C(N)=O)c(Nc4ccc(C5CCN(CC6CCN(c7ccc(C(=O)NC8CCC(=O)NC8=O)cc7)CC6)CC5)cc4)n3)C2)C1=O] 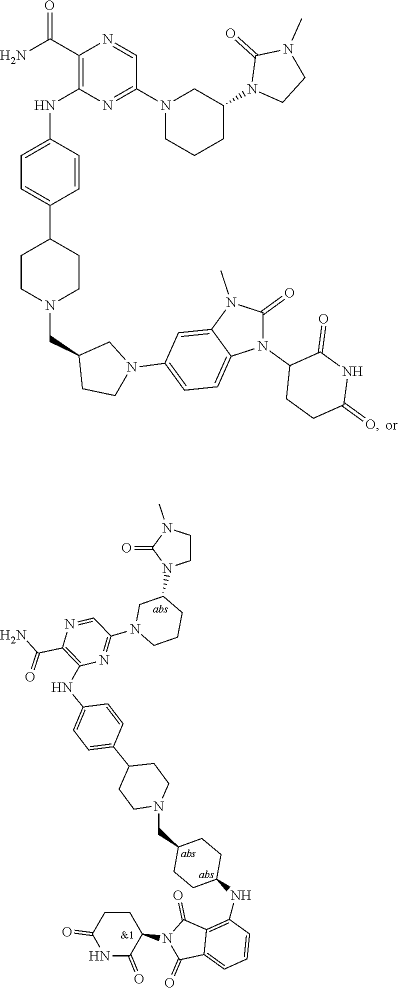
or a pharmaceutically acceptable salt thereof.

126. A pharmaceutical composition comprising the compound of claim 125 and one or more pharmaceutically acceptable carriers, excipients, or diluents.

127. The method of any of claims 1-124 wherein the compound or pharmaceutical composition is according to claim 125 or 126.
